(19) United States
(12) Patent Application Publication
(10) Pub. No.: US 2014/0309184 A1
(43) Pub. Date: Oct. 16, 2014

(54) METHODS AND COMPOSITIONS FOR THE TREATMENT OF OVARIAN CANCER

(71) Applicants: Rodney P. Rocconi, Mobile, AL (US); Lalita Samant, Mobile, AL (US)

(72) Inventors: Rodney P. Rocconi, Mobile, AL (US); Lalita Samant, Mobile, AL (US)

(73) Assignee: UNIVERSITY OF SOUTH ALABAMA, Mobile, AL (US)

(21) Appl. No.: 14/346,186

(22) PCT Filed: Jul. 20, 2012
(86) PCT No.: PCT/US12/47713

§ 371 Date: Jun. 24, 2014

Related U.S. Application Data
(60) Provisional application No. 61/537,521, filed on Sep. 21, 2011.

(51) Int. Cl. A61K 31/517; A61K 31/5377; A61K 31/553; A61K 31/704; A61K 31/337; A61K 31/7068; A61K 31/475; A61K 31/282; A61K 31/55

(52) U.S. Cl. CPC A61K 31/517; A61K 31/282; A61K 31/5377; A61K 31/553; A61K 31/55; A61K 31/337; A61K 31/7068; A61K 31/475; A61K 31/704
USPC 514/34; 514/266.4; 514/266.2; 514/234.5; 514/266.22; 514/252.17; 514/266.23; 514/266.24; 514/211.09; 514/217.06; 514/210.18; 514/49; 435/375

(57) ABSTRACT

Embodiments of the present disclosure relate to methods and compositions for treating a subject with ovarian cancer. Some embodiments include treating a subject with a particular combination of chemotherapeutic agents.

### RELATED APPLICATIONS

This application claims the benefit of U.S. Provisional Application No. 61/537,521 entitled “METHODS AND COMPOSITIONS FOR THE TREATMENT OF OVARIAN CANCER” filed on Sep. 21, 2011, the entire disclosure of which is incorporated by reference in its entirety.

### PARTIES OF JOINT RESEARCH AGREEMENT

The inventions described herein were made as a result of activities undertaken within the scope of a Jun. 9, 2009, Joint Research Agreement between the University of South Alabama, Bristol-Myers Squibb Company, Lalita S. Samant, Ph.D., and Rodney P. Rocconi, M.D.

### FIELD OF THE INVENTION

Embodiments of the present disclosure relate to methods and compositions for treating a subject with ovarian cancer. Some embodiments include treating a subject with a particular combination of chemotherapeutic agents.

### BACKGROUND

Greater than 20,000 women are diagnosed with ovarian cancer each year in the United States. Although the majority of patients present with advanced disease, most will respond to cytoreductive surgery and first line platinum-based combination chemotherapy. Despite this response, the majority of patients ultimately experience a recurrence and unfortunately succumb to progressive disease.

Paramount to the prognosis of these patients is the disease's varying sensitivity to platinum agents. Although a continuum, patients are stratified by their disease's original response to platinum (i.e. disease-free interval). Patients are classified as “platinum-sensitive” (>6 months after initial platinum agent) and “platinum-resistant” (<6 months disease-free interval) the length of relapse-free interval after platinum agents.

Platinum-sensitive patients have a more favorable prognosis and are more likely to respond to subsequent therapy at the time of relapse. Furthermore, the disease-free interval is highly predictive of the overall response rate and complete response rate. For example, patients with a disease-free interval of 6 to 12 months can have an overall response rate=27% and complete response rate=5%; patients with a disease-free interval of 13 to 24 months can have an overall response rate=33% and complete response rate=11%; and patients with a disease-free interval>24 months can have an overall response rate=59% and complete response rate=22%.

In contrast, patients with platinum-resistant disease by definition relapse less than six months after completion of prior platinum therapy and characteristically have stable disease as the best response and do not generally respond to second-line platinum-based therapy. Their prognosis remains poor with a median overall survival usually less than 12 months. Accordingly, there is a need for improved therapies to treat cancers, such as ovarian cancer.

### SUMMARY

Some embodiments of the methods, compositions, uses and kits provided herein include a method of killing or retarding the growth of a neoplastic cell comprising: contacting the cell with an effective amount of a SMO inhibitor in combination with an effective amount of a chemotherapeutic agent.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor.

In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

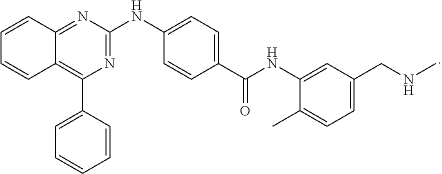 [SMILES: CNCc1ccc(C)c(NC(=O)c2ccc(Nc3nc(-c4ccccc4)c4ccccc4n3)cc2)c1]

In some embodiments, the cell is contacted with the BMS-833923 and the chemotherapeutic agent sequentially.

In some embodiments, contacting the cell with the chemotherapeutic agent commences before contacting the cell with the BMS-833923.

In some embodiments, contacting the cell with the chemotherapeutic agent is before contacting the cell with the BMS-833923. In some embodiments, contacting the cell with the chemotherapeutic agent is less than 48 hours prior to contacting the cell with the BMS-833923. In some embodiments, contacting the cell with the chemotherapeutic agent is less than 36 hours prior to contacting the cell with the BMS-833923. In some embodiments, contacting the cell with the chemotherapeutic agent is less than 24 hours prior to contacting the cell with the BMS-833923. In some embodiments, contacting the cell with the chemotherapeutic agent is less than 12 hours prior to contacting the cell with the BMS-833923. In some embodiments, contacting the cell with the chemotherapeutic agent is less than 6 hours prior to contacting the cell with the BMS-833923.

In some embodiments, the chemotherapeutic agent and the BMS-833923 are contacted with the cell simultaneously.

In some embodiments, contacting the cell with the chemotherapeutic agent commences after contacting the cell with the BMS-833923.

In some embodiments, the chemotherapeutic agent is selected from the group consisting of cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin. In some embodiments, the chemotherapeutic agent is selected from the group consisting of taxol, gemcitabine, topotecan hydrochloride, doxorubicin and pegylated doxorubicin.

In some embodiments, the cell is contacted in vitro.

In some embodiments, the cell is contacted in vivo.

In some embodiments, the cell comprises an ovarian cancer cell. In some embodiments, the cell comprises a platinum resistant ovarian cancer cell. In some embodiments, the cell comprises an ovarian cancer stem cell.

In some embodiments, the cell is mammalian. In some embodiments, the cell is human.

Some embodiments of the methods, compositions, kits and uses provided herein include a method of increasing the sensitivity of a neoplastic cell to a chemotherapeutic compound comprising contacting the cell with an effective amount a SMO inhibitor and an effective amount of the chemotherapeutic compound, wherein the effective amount of the chemotherapeutic compound is significantly less than the effective amount of the chemotherapeutic compound in the absence of the SMO inhibitor.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor. In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

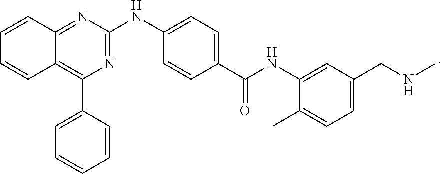 [SMILES: CNCc1ccc(C)c(NC(=O)c2ccc(Nc3nc(-c4ccccc4)c4ccccc4n3)cc2)c1]

In some embodiments, the chemotherapeutic agent is selected from the group consisting of cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin. In some embodiments, the chemotherapeutic agent is selected from the group consisting of taxol, gemcitabine, topotecan hydrochloride, doxorubicin and pegylated doxorubicin.

In some embodiments, the cell is contacted in vitro.

In some embodiments, the cell is contacted in vivo.

In some embodiments, the cell comprises an ovarian cancer cell. In some embodiments, the cell comprises a platinum resistant ovarian cancer cell. In some embodiments, the cell comprises an ovarian cancer stem cell.

In some embodiments, the cell is mammalian. In some embodiments, the cell is human.

Some embodiments of the methods, compositions, kits and use provided herein include a method of ameliorating cancer in a subject comprising: administering to the subject an effective amount of a SMO inhibitor in combination with an effective amount of a chemotherapeutic agent.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor.

In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

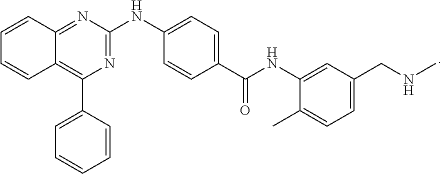 [SMILES: CNCc1ccc(C)c(NC(=O)c2ccc(Nc3nc(-c4ccccc4)c4ccccc4n3)cc2)c1]

In some embodiments, the BMS-833923 and chemotherapeutic agent are administered sequentially.

In some embodiments, administration of the chemotherapeutic agent to the subject commences before administration of the BMS-833923 to the subject.

In some embodiments, the chemotherapeutic agent is administered to the subject before the administration of the BMS-833923 to the subject. In some embodiments, the chemotherapeutic agent is administered to the subject less than 48 hours prior to administering the BMS-833923 to the subject. In some embodiments, the chemotherapeutic agent is administered to the subject less than 36 hours prior to administering the BMS-833923 to the subject. In some embodiments, the chemotherapeutic agent is administered to the subject less than 24 hours prior to administering the BMS-833923 to the subject. In some embodiments, the chemotherapeutic agent is administered to the subject less than 12 hours prior to administering the BMS-833923 to the subject. In some embodiments, the chemotherapeutic agent is administered to the subject less than 6 hours prior to administering the BMS-833923 to the subject.

In some embodiments, the chemotherapeutic agent and the BMS-833923 are administered simultaneously to the subject.

In some embodiments, the chemotherapeutic agent is administered to the subject after administering the BMS-833923 to the subject.

In some embodiments, the BMS-833923 is administered at least about daily. In some embodiments, the BMS-833923 is administered at least about weekly.

In some embodiments, a dose of at least about 1 mg BMS-833923 is administered to the subject. In some embodiments, a dose of at least about 5 mg BMS-833923 is administered to the subject. In some embodiments, a dose of at least about 10 mg BMS-833923 is administered to the subject. In some embodiments, a dose of at least about 20 mg BMS-833923 is administered to the subject. In some embodiments, a dose of at least about 30 mg BMS-833923 is administered to the subject.

In some embodiments, the BMS-833923 is administered orally.

In some embodiments, the chemotherapeutic agent is selected from the group consisting cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin. In some embodiments, the chemotherapeutic agent is selected from the group consisting of taxol, gemcitabine, topotecan hydrochloride, doxorubicin and pegylated doxorubicin.

In some embodiments, the chemotherapeutic agent is administered daily.

In some embodiments, the chemotherapeutic agent is administered weekly.

In some embodiments, the chemotherapeutic agent is administered intravenously.

In some embodiments, the cancer comprises ovarian cancer. In some embodiments, the cancer comprises a platinum resistant ovarian cancer cell. In some embodiments, the cancer comprises an ovarian cancer stem cell.

In some embodiments, the subject is mammalian. In some embodiments, the subject is human.

Some embodiments of the methods, compositions, kits and uses provided herein include a method for reducing the dosage of a chemotherapeutic agent needed to ameliorate cancer in a subject comprising administering an effective amount of a SMO inhibitor and administering the chemotherapeutic agent to the subject.

Some embodiments of the methods, compositions, kits and uses provided herein include a method for increasing the sensitivity of a cancer to a chemotherapeutic compound comprising contacting the cancer with an effective amount a SMO inhibitor and an effective amount of the chemotherapeutic compound, wherein the effective amount of the chemotherapeutic compound is significantly less than the effective amount of the chemotherapeutic compound in the absence of the SMO inhibitor.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor.

In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

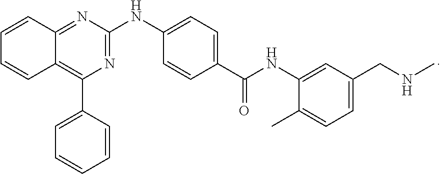 [SMILES: CNCc1ccc(C)c(NC(=O)c2ccc(Nc3nc(-c4ccccc4)c4ccccc4n3)cc2)c1]

In some embodiments, the BMS-833923 and chemotherapeutic agent are administered sequentially.

In some embodiments, administration of the chemotherapeutic agent to the subject commences before administration of the BMS-833923 to the subject.

In some embodiments, the chemotherapeutic agent is administered to the subject before the administration of the BMS-833923 to the subject.

In some embodiments, the chemotherapeutic agent is administered to the subject less than 48 hours prior to administering the BMS-833923 to the subject. In some embodiments, the chemotherapeutic agent is administered to the subject less than 36 hours prior to administering the BMS-833923 to the subject. In some embodiments, the chemotherapeutic agent is administered to the subject less than 24 hours prior to administering the BMS-833923 to the subject. In some embodiments, the chemotherapeutic agent is administered to the subject less than 12 hours prior to administering the BMS-833923 to the subject. In some embodiments, the chemotherapeutic agent is administered to the subject less than 6 hours prior to administering the BMS-833923 to the subject.

In some embodiments, the chemotherapeutic agent and the BMS-833923 are administered simultaneously to the subject.

In some embodiments, administration of the chemotherapeutic agent to the subject commences before administration of the BMS-833923 to the subject.

In some embodiments, the chemotherapeutic agent is administered to the subject after the administration of the BMS-833923 to the subject.

In some embodiments, the BMS-833923 is administered at least about daily. In some embodiments, the BMS-833923 is administered at least about weekly.

In some embodiments, a dose of at least about 1 mg BMS-833923 is administered to the subject. In some embodiments, a dose of at least about 5 mg BMS-833923 is administered to the subject. In some embodiments, a dose of at least about 10 mg BMS-833923 is administered to the subject. In some embodiments, a dose of at least about 20 mg BMS-833923 is administered to the subject. In some embodiments, a dose of at least about 30 mg BMS-833923 is administered to the subject.

In some embodiments, the BMS-833923 is administered orally.

In some embodiments, the chemotherapeutic agent is selected from the group consisting of cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin.

In some embodiments, the chemotherapeutic agent is selected from the group consisting of taxol, gemcitabine, topotecan hydrochloride, doxorubicin and pegylated doxorubicin.

In some embodiments, the chemotherapeutic agent is administered daily.

In some embodiments, the chemotherapeutic agent is administered weekly.

In some embodiments, the chemotherapeutic agent is administered intravenously.

In some embodiments, the cancer comprises ovarian cancer. In some embodiments, the cancer comprises a platinum resistant ovarian cancer cell. In some embodiments, the cancer comprises an ovarian cancer stem cell.

In some embodiments, the subject is mammalian. In some embodiments, the subject is human.

Some embodiments of the methods, compositions, kits and uses provided herein include a method for determining whether a candidate agent for ameliorating cancer acts in synergy with a SMO inhibitor comprising contacting a population of cells with a SMO inhibitor in combination with a test compound; and determining whether the level of cell survival in the population of cells contacted with the SMO inhibitor in combination with the test compound is significantly less than the combined level of cell survival in a population of cells contacted with the SMO inhibitor and a population of cells contacted with the test compound.

In some embodiments, a significantly lower level of cell survival in the population of cells contacted with the SMO inhibitor in combination with the test compound compared to the combined level of cell survival in a population of cells contacted with the SMO inhibitor and a population of cells contacted with the test compound indicates that the test compound acts in synergy with the SMO inhibitor.

In some embodiments, the SMO inhibitor is selected from a compound of Table 3. In some embodiments, the SMO inhibitor comprises BMS-833923, having the structure:

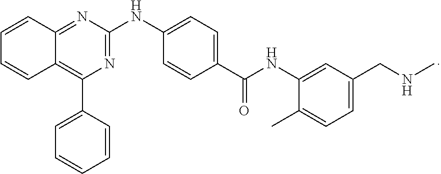 [SMILES: CNCc1ccc(C)c(NC(=O)c2ccc(Nc3nc(-c4ccccc4)c4ccccc4n3)cc2)c1]

In some embodiments, the significantly lower level of cell survival comprises a decrease of at least about 10% relative to the combined level of cell survival. In some embodiments, the significantly lower level of cell survival comprises a decrease of at least about 20% relative to the combined level of cell survival. In some embodiments, the significantly lower level of cell survival comprises a decrease of at least about 30% relative to the combined level of cell survival. In some embodiments, the significantly lower level of cell survival comprises a decrease of at least about 40% relative to the combined level of cell survival.

In some embodiments, each population of cells is mammalian. In some embodiments, each population of cells is human.

In some embodiments, each population of cells comprises cancer cells. In some embodiments, each population of cells comprises ovarian cancer cells. In some embodiments, each population of cells comprises platinum resistant ovarian cancer cells. In some embodiments, each population of cells comprises ovarian cancer stem cells.

Some embodiments also include preparing a pharmaceutical composition comprising the test compound which acts in synergy with the SMO inhibitor.

In some embodiments, the pharmaceutical composition is suitable for intravenous administration.

In some embodiments, the pharmaceutical composition is a pill.

Some embodiments include a kit for treating ovarian cancer in a subject comprising a SMO inhibitor and a chemotherapeutic agent.

In some embodiments, the chemotherapeutic agent comprises a platinum-based chemotherapeutic agent.

In some embodiments, the SMO inhibitor is selected from a compound of Table 3. In some embodiments, the SMO inhibitor comprises BMS-833923, having the structure:

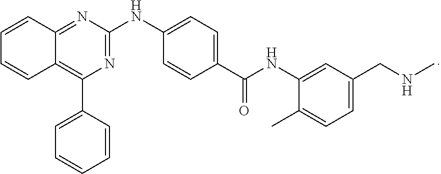 [SMILES: CNCc1ccc(C)c(NC(=O)c2ccc(Nc3nc(-c4ccccc4)c4ccccc4n3)cc2)c1]

Some embodiments also include a pharmaceutical carrier.

Some embodiments also include an instrument for administering the SMO inhibitor or chemotherapeutic agent to the subject.

Some embodiments include use of an effective amount of a SMO inhibitor in combination with an effective amount of a chemotherapeutic agent for ameliorating cancer in a subject in need thereof.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor.

In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

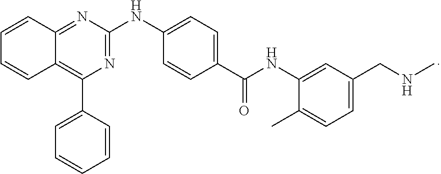 [SMILES: CNCc1ccc(C)c(NC(=O)c2ccc(Nc3nc(-c4ccccc4)c4ccccc4n3)cc2)c1]

In some embodiments, the BMS-833923 and chemotherapeutic agent are administered sequentially.

In some embodiments, administration of the chemotherapeutic agent to the subject commences before administration of the BMS-833923 to the subject.

In some embodiments, the chemotherapeutic agent is administered to the subject before the administration of the BMS-833923 to the subject.

In some embodiments, the chemotherapeutic agent is administered to the subject less than 48 hours prior to administering the BMS-833923 to the subject. In some embodiments, the chemotherapeutic agent is administered to the subject less than 36 hours prior to administering the BMS-833923 to the subject. In some embodiments, the chemotherapeutic agent is administered to the subject less than 24 hours prior to administering the BMS-833923 to the subject. In some embodiments, the chemotherapeutic agent is administered to the subject less than 12 hours prior to administering the BMS-833923 to the subject. In some embodiments, the chemotherapeutic agent is administered to the subject less than 6 hours prior to administering the BMS-833923 to the subject.

In some embodiments, the chemotherapeutic agent and the BMS-833923 are administered simultaneously to the subject.

In some embodiments, the chemotherapeutic agent is administered to the subject after administering the BMS-833923 to the subject.

In some embodiments, the BMS-833923 is administered at least about daily.

In some embodiments, the BMS-833923 is administered at least about weekly.

In some embodiments, a dose of at least about 1 mg BMS-833923 is administered to the subject. In some embodiments, a dose of at least about 5 mg BMS-833923 is administered to the subject. In some embodiments, a dose of at least about 10 mg BMS-833923 is administered to the subject. In some embodiments, a dose of at least about 20 mg BMS-833923 is administered to the subject. In some embodiments, a dose of at least about 30 mg BMS-833923 is administered to the subject.

In some embodiments, the BMS-833923 is administered orally.

In some embodiments, the chemotherapeutic agent is selected from the group consisting cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin.

In some embodiments, the chemotherapeutic agent is selected from the group consisting of taxol, gemcitabine, topotecan hydrochloride, doxorubicin and pegylated doxorubicin.

In some embodiments, the chemotherapeutic agent is administered daily.

In some embodiments, the chemotherapeutic agent is administered weekly.

In some embodiments, the chemotherapeutic agent is administered intravenously.

In some embodiments, the cancer comprises ovarian cancer. In some embodiments, the cancer comprises a platinum resistant ovarian cancer cell. In some embodiments, the cancer comprises an ovarian cancer stem cell.

In some embodiments, the subject is mammalian. In some embodiments, the subject is human.

Some embodiments include use of an effective amount of a SMO inhibitor for reducing the dosage of a chemotherapeutic agent needed to ameliorate cancer in a subject.

Some embodiments include use of an effective amount a SMO inhibitor for increasing the sensitivity of a cancer to a chemotherapeutic compound, wherein the effective amount of the chemotherapeutic compound is significantly less than the effective amount of the chemotherapeutic compound in the absence of the SMO inhibitor.

In some embodiments, the chemotherapeutic agent comprises a platinum-based chemotherapeutic agent.

In some embodiments, the effective amount of the chemotherapeutic compound is at least about 10% less than the effective amount of the chemotherapeutic compound in the absence of the SMO inhibitor. In some embodiments, the effective amount of the chemotherapeutic compound is at least about 20% less than the effective amount of the chemotherapeutic compound in the absence of the SMO inhibitor. In some embodiments, the effective amount of the chemotherapeutic compound is at least about 30% less than the effective amount of the chemotherapeutic compound in the absence of the SMO inhibitor. In some embodiments, the effective amount of the chemotherapeutic compound is at least about 40% less than the effective amount of the chemotherapeutic compound in the absence of the SMO inhibitor. In some embodiments, the effective amount of the chemotherapeutic compound is at least about 50% less than the effective amount of the chemotherapeutic compound in the absence of the SMO inhibitor.

In some embodiments, the effective amount of the SMO inhibitor is an amount which significantly reduces the IC50 of the chemotherapeutic compound. In some embodiments, the IC50 of the chemotherapeutic agent is reduced by at least about 20%. In some embodiments, the IC50 of the chemotherapeutic agent is reduced by at least about 30%. In some embodiments, the IC50 of the chemotherapeutic agent is reduced by at least about 40%. In some embodiments, the IC50 of the chemotherapeutic agent is reduced by at least about 50%.

In some embodiments, the effective amount of the chemotherapeutic compound and the effective amount of the SMO inhibitor have a Combination Index less than 1 as determined by Calcusyn software.

In some embodiments, the SMO inhibitor is selected from a compound of Table 3. In some embodiments, the SMO inhibitor comprises BMS-833923, having the structure:

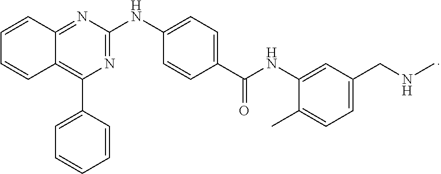 [SMILES: CNCc1ccc(C)c(NC(=O)c2ccc(Nc3nc(-c4ccccc4)c4ccccc4n3)cc2)c1]

In some embodiments, the BMS-833923 and chemotherapeutic agent are administered sequentially.

In some embodiments, administration of the chemotherapeutic agent to the subject commences before administration of the BMS-833923 to the subject.

In some embodiments, the chemotherapeutic agent is administered to the subject before the administration of the BMS-833923 to the subject.

In some embodiments, the chemotherapeutic agent is administered to the subject less than 48 hours prior to administering the BMS-833923 to the subject. In some embodiments, the chemotherapeutic agent is administered to the subject less than 36 hours prior to administering the BMS-833923 to the subject. In some embodiments, the chemotherapeutic agent is administered to the subject less than 24 hours prior to administering the BMS-833923 to the subject. In some embodiments, the chemotherapeutic agent is administered to the subject less than 12 hours prior to administering the BMS-833923 to the subject. In some embodiments, the chemotherapeutic agent is administered to the subject less than 6 hours prior to administering the BMS-833923 to the subject.

In some embodiments, the chemotherapeutic agent and the BMS-833923 are administered simultaneously to the subject.

In some embodiments, administration of the chemotherapeutic agent to the subject commences before administration of the BMS-833923 to the subject.

In some embodiments, the chemotherapeutic agent is administered to the subject after the administration of the BMS-833923 to the subject.

In some embodiments, the BMS-833923 is administered at least about daily.

In some embodiments, the BMS-833923 is administered at least about weekly.

In some embodiments, a dose of at least about 1 mg BMS-833923 is administered to the subject. In some embodiments, a dose of at least about 5 mg BMS-833923 is administered to the subject. In some embodiments, a dose of at least about 10 mg BMS-833923 is administered to the subject. In some embodiments, a dose of at least about 20 mg BMS-833923 is administered to the subject. In some embodiments, a dose of at least about 30 mg BMS-833923 is administered to the subject.

In some embodiments, the BMS-833923 is administered orally.

In some embodiments, the chemotherapeutic agent is selected from the group consisting of cisplatin, carboplatin, nedaplatin, oxaliplatin, satraplatin, and triplatin tetranitrate. In some embodiments, the chemotherapeutic agent comprises carboplatin. In some embodiments, the chemotherapeutic agent comprises gemcitabine. In some embodiments, the chemotherapeutic agent comprises topotecan hydrochloride. In some embodiments, the chemotherapeutic agent comprises pegylated doxorubicin. In some embodiments, the chemotherapeutic agent is selected from the group consisting of taxol, gemcitabine, topotecan hydrochloride, doxorubicin and pegylated doxorubicin.

In some embodiments, the chemotherapeutic agent is administered daily.

In some embodiments, the chemotherapeutic agent is administered weekly.

In some embodiments, the chemotherapeutic agent is administered intravenously.

In some embodiments, the cancer comprises ovarian cancer. In some embodiments, the cancer comprises a platinum resistant ovarian cancer cell. In some embodiments, the cancer comprises an ovarian cancer stem cell.

In some embodiments, the subject is mammalian. In some embodiments, the subject is human.

Some embodiments include a composition comprising a SMO inhibitor and a chemotherapeutic agent in a pharmaceutically acceptable carrier.

In some embodiments, the SMO inhibitor is selected from a compound of Table 3. In some embodiments, the SMO inhibitor comprises BMS-833923, having the structure:

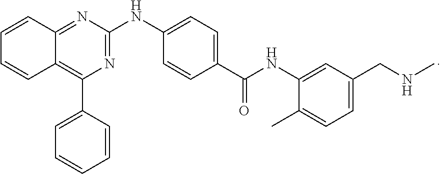 [SMILES: CNCc1ccc(C)c(NC(=O)c2ccc(Nc3nc(-c4ccccc4)c4ccccc4n3)cc2)c1]

In some embodiments, the chemotherapeutic agent is selected from the group consisting of cisplatin, carboplatin, nedaplatin, oxaliplatin, satraplatin, and triplatin tetranitrate. In some embodiments, the chemotherapeutic agent is selected from the group consisting of taxol, gemcitabine, topotecan hydrochloride, doxorubicin and pegylated doxorubicin.

In some embodiments, the composition includes a pill, tablet, or powder.

In some embodiments, the composition includes a solution.

### BRIEF DESCRIPTION OF THE DRAWINGS

FIG. 1 is a graph of the percentage cell survival for enriched CD44+/CD24− cells compared to other phenotypes.

FIG. 2 depicts a Western blot of ES2, SKOV3, OV90, and TOV112D lysates probed for various proteins.

FIG. 3A depicts a series of graphs of percentage survival of ES2 cells (top left panel), SKOV3 cells (top right panel), OV90 cells (bottom left panel), and TOV112D cells (bottom right panel) treated with various concentrations of BMS-833923. FIG. 3B depicts graphs of percentage survival of SKOV3 cells, ES2 cells, and TOV112D cells treated with various concentrations of cyclopamine.

FIG. 4 depicts a graph of fold change in expression of GLI and PTCH in SKOV3 ovarian cancer cell line was treated with 2.5 μM of BMS-833923

FIG. 5 depicts graphs for SKOV3 cells (top panel) and OV90 cells (bottom panel) for number of cells invaded for non ovarian stem cells (non-OSC), CD44+CD24− ovarian stem cells, and CD44+CD24− ovarian stem cells treated with 2.5 μM BMS-833923.

FIG. 6 depicts a series of immunofluorescent photomicrographs of untreated SKOV3 cells stained with DAPI (top left panel) or FITC-labeled Gli1 (top right panel), and SKOV3 cells treated with BMS-833923 stained with DAPI (bottom left panel) or FITC-labeled Gli1 (bottom right panel).

FIGS. 7A, 7B, 7C, and 7D depict percentage cell survival for SKOV3 cells, TOV112D cells, ES2 cells, and OC90 cells, respectively, each cell line treated with various combinations of BMS-833923, Carboplatin, Taxol, Gemzar, Topotecan, and Doxil. The starred column in FIG. 7A indicates synergy.

FIG. 8 depicts a graph of percentage cell survival for SKOV3 cells treated with various combinations of BMS-833923, Carboplatin, and Taxol.

FIG. 9 depicts a graph of Combination Index (CI) for various fractions of cells affected by BMS-833923 in combination with Carboplatin.

FIG. 10 depicts a graph of percentage cell survival for SKOV3 cells treated sequentially with either BMS-833923 and then Carboplatin, or Carboplatin and then BMS-833923.

FIG. 11A depicts a graph of percentage cell survival for A2780 cells or A2780/CP70 cells treated with various concentrations of Carboplatin at 24 hr, 48 hr, and 72 hr. FIG. 11B depicts a graph of percentage cell survival for A2780 cells treated with various concentrations of Carboplatin with and without 5.0 μM BMS-833923 at 24 hr, 48 hr, and 72 hr. FIG. 11C depicts a graph of percentage cell survival for A2780/CP70 cells treated with various concentrations of Carboplatin with and without 5.0 μM BMS-833923 at 24 hr, 48 hr, and 72 hr.

FIG. 12A and FIG. 12B depict graphs relating to in vivo assessments of BMS-833923 and Carboplatin as single agents and in combination therapy in the carboplatin-sensitive A2780 ovarian tumor model and carboplatin-resistant CP2780 ovarian tumor model, respectively.

FIG. 13A and FIG. 13B depict graphs relating to percentage weight change of xenograft tumors in mice treated with BMS-833923 and/or Carboplatin.

FIG. 14 depicts graphs of the relative levels of mRNA expression in SKOV3 cells treated with various concentrations of carboplatin for SHH (top left panel), Smo (top right panel), PTCH1 (bottom left panel), and Gli1 (bottom right panel).

### DETAILED DESCRIPTION

Embodiments of the present disclosure relate to methods and compositions for treating a subject with ovarian cancer. Some embodiments include treating a subject with a particular combination of chemotherapeutic agents. One strategy to improve success of ovarian cancer therapy is to enhance a cancer's sensitivity to platinum-based chemotherapeutic agents. If such chemoresistance could be overcome, response rates, overall survival and cure rates would significantly improve. Numerous investigations using either alternative chemotherapy agents, such as, various combinations and schedules and/or targeted biologic therapy, have failed to enhance ovarian cancer's platinum sensitivity to date.

One hypothesis associated with platinum-resistant ovarian cancer and its poor prognosis revolves around cancer stem cells. The origin of cancer from “stem cell” populations was first introduced approximately 150 years ago. The conceptual basis for a stem cell origin of cancer is supported by observations that certain sub-populations of cancer cells appear to acquire stem cell-like properties such as the capacity of self-renewal, and the ability to differentiate. Additionally, cancer stem cells possess an innate resistance to cytotoxic agents. Irrespective of response rates, if chemotherapy fails to eradicate cancer stem cells then cancer may regenerate and a recurrence or progression of disease can occur.

One important cancer stem cell marker phenotype CD44+/CD24− was originally described in breast cancer stem cells and recently has been implicated to be an ovarian cancer stem cell marker with self-renewal and chemoresistance properties. The ovarian cancer CD44+/CD24− phenotype correlates to in vitro aggressiveness and has the properties of enhanced differentiation, invasion, and resistance to chemotherapy. Furthermore, this CD44+/CD24− phenotype appeared to correlate to prognosis clinically with an increase risk of recurrence and shorter progression-free survivals in ovarian cancer patients. Ascites derived from 20 advanced stage ovarian cancer patients was obtained and evaluated for the proportion of CD44+/CD24− cells and its correlation to survival. After confirmation by CK7 immunofluorescent staining, ovarian cancer cells obtained from ascites were evaluated for proportion of CD44+/CD24− cells (range 3.7 to 97.7%). Using a threshold of 25% CD44+/CD24− cells, patients with >25% ovarian cancer stem cells (OCSC) were significantly more likely to recur (83 vs. 14%, p=0.003) and had shorter median progression-free survival (6 vs. 18 months, p=0.01)

Determining the innate molecular differences between CD44+/CD24− ovarian cancer stem cells (OCSC) and non-stem cell ovarian cancer is an important goal and could lead to important discoveries in targeted therapy. One molecular pathway of interest includes the Hedgehog (Hh) pathway. Normally dormant, the Hh signaling pathway has shown enhanced activity in numerous malignancies including ovarian cancer as well as a well-documented role in cancer stem cells.

Activation of Hh represented by the translocation of GL11 to the nucleus is initiated by the cell surface protein, smoothened (SMO). Inhibition of this pathway via the SMO inhibitor, N-{2-methyl-5-[(methylamino)methyl]phenyl}-4-[(4-phenylquinazolin-2-yl)amino]benzamide (also known as BMS-833923 or XL139), resulted in a significant downregulation of GLl1 (5-fold) and PTCH (3-fold) proteins compared to untreated controls (See e.g., U.S. Pat. No. 8,222,263; Siu L. et al., J. Clin. Oncol. 2010; 28:15s (suppl; abstr 2501); and National Institute of Health Clinical Trial Identifier No. NCT006701891, NCT00909402, NCT00927875, NCT01218477, NCT01357655 and NCT01413906, the disclosures of which are incorporated herein by reference in their entireties). The structure of BMS-833923 is:

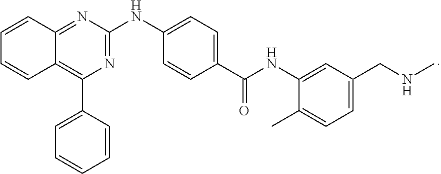 [SMILES: CNCc1ccc(C)c(NC(=O)c2ccc(Nc3nc(-c4ccccc4)c4ccccc4n3)cc2)c1]

Further immunofluorescent-staining of GLl1 showed near complete exclusion of intranuclear GLl1 with nuclear shadowing and vacuolization. MTS assays were also performed to assess the effects of a combination of chemotherapy plus BMS-833923 on cell survival. A MTS assay is a colorimetric assay to assess cell viability. MTS assay is composed of solutions of a novel tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent phenazine methosulfate (PMS). MTS is bio-reduced by cells into a formazan product that is soluble in tissue culture medium. The absorbance of the formazan product at 490 nm can be measured directly from 96-well assay plates without additional processing. The conversion of MTS into the aqueous soluble formazan product is accomplished by dehydrogenase enzymes found in metabolically active cells. The quantity of formazan product as measured by the amount of 490 nm absorbance is directly proportional to the number of living cells in culture. In MTS cell survival assays, a combination of chemotherapy plus BMS-833923 was more effective compared to chemotherapy or BMS-833923 alone. Specifically, significant cell death was demonstrated with the combination of BMS-833923 and carboplatin (8% cell survival). Calcusyn CI calculations determined that multiple dosage combinations resulted in synergy with CI range of 0.3 to 0.49 (synergy defined as CI<1).

The effect of inhibition of Hh pathway on platinum resistant ovarian cancer cells was also determined. Ovarian cancer cells engineered to be platinum resistant (A2780-CP70) were observed to be 6-fold more sensitive to platinum when inhibiting the hedgehog pathway.

A strategy to improve success of ovarian cancer therapy would be to enhance a cancer's sensitivity to platinum-based chemotherapeutic agents. If chemoresistance could be overcome, response rates, overall survival and cure rates would significantly improve. As described herein, inhibiting the hedgehog pathway improved the sensitivity of platinum-resistant ovarian cancer to platinum-based chemotherapeutic agents 6-fold. This discovery could significantly enhance the effectiveness of the treatment of patients with ovarian cancer.

Overcoming platinum-sensitivity is important in improving ovarian cancer survival. Ovarian cancer stem cells and specifically the hedgehog pathway contribute to resistance to therapy. Reversing platinum-resistance and/or sensitizing ovarian cancer to platinum agents can directly improve patient outcomes. The methods described herein may improve response rates, overall survival and cure rates, offer treatment options to group of patients with less favorable prognosis, and provide a reduction in doses typically used with platinum-based chemotherapeutic agents, thereby reducing systemic side effects and toxicities.

### Hedgehog Signaling

The Hedgehog pathway is aberrantly active in several cancer types, including breast cancer and melanoma (Xuan, et al. (2009) J. Cancer Res. Clin. Oncol. 135, 235-240). Hedgehog pathway components were detected in nevi, melanoma, and lymph node metastases of melanoma (Stecca, B., et al. (2007) Proc. Natl. Acad. Sci. U.S.A. 104, 5895-5900). Cells may develop resistance to a range of chemotherapeutic compounds by reverting to a stem cell-like state. Dysregulation of the Hedgehog pathway contributes to uncontrolled growth of some tumors and resistance to chemotherapy treatments (Rubin L L et al. “Targeting the hedgehog pathway in cancer.” Nat Rev Drug Discov. 2006; 5 (12):1026-1033; and Sims-Mourtada J, et al. “Sonic hedgehog promotes multiple drug resistance by regulation of drug transport.” Oncogene. 2007; 26 (38):5674-5679). The Hedgehog pathway including genes such as Gli1 play a role in the development and maintenance of a stem cell-like phenotype (Peacock C D, et al. Proc Natl Acad Sci USA 104:4048-4053, 2007). The Hh pathway may be a regulator of cancer stem cells. A number of human cancers are associated with mutations in the Hh pathway, overexpression of pathway components, or cancer stem cells with Hh pathway activation. Signaling through the Hh pathway involves Smoothened (SMO), a receptor associated with initiation of growth signals, and Patched 1, a receptor that inhibits signaling by SMO.

### Cancer Stem Cells and Drug Resistance

Human ovarian cancer cells grown under conditions that support a sub-population that grows in spheroids selects for cells that have a more potent ability to form new independent cancers (Zhang S, et al. Cancer Res 68:4311-4320, 2008). As few as 100 spheroid forming cells could form new independent tumors when transferred to nude mice, while as many as 100,000 cells grown in monolayer, were unable to form independent tumors. The cancer initiating cells (cancer stem cells; CSCs) become much more drug resistant to a variety of agents, including platinum-based chemotherapeutic compounds such as cisplatin and paclitaxel. Such cells may also express a set of molecular markers that differ from the same cell line, grown in monolayer, such as CD117, CD44, and Nestin. Cancer initiating cells have been investigated in other malignancies including prostate cancer, breast cancer, and lung cancer (Zietarska M, et al. Molecular Carcinogenesis 46:872-885, 2007; Burleson K et al. Gynecologic Oncology 93: 170-181, 2004; Casey R C, et al. Am J of Pathology 159:2071-2080, 2001).

In cancer types where neoplastic growth and differentiation depend on CSCs, complete eradication of this population may be curative. Furthermore, agents that force CSCs to rapidly differentiate en masse within such cancer types may limit disease progression. Alternatively, suppressing residual CSCs after initial tumor debulking may sustain remissions and extend the progression-free survival of patients receiving CSC suppressive therapy. Considering these distinct therapeutic potentials of targeting CSCs, it appears that CSC-targeted therapies could be an effective complement to traditional treatment approaches such as surgery, chemotherapy, and radiation therapy. Indeed, it is possible that these traditional strategies leave behind residual CSCs which are capable of spreading and regenerating tumors, leading to cancer recurrence and metastasis. Moreover, these recurring tumors often acquire resistance to chemotherapy and radiation.

Ovarian cancer stem cells may be responsible for persistent low volume disease after induction of a clinical complete response. The inability to eradicate such cells may be a function of cell dormancy, the relative inability of any chemotherapy to have a meaningful effect on cells in the dormant state or these cells may represent a state of extreme drug resistance at the molecular level.

Ovarian cancer stem cells include, in order of increasing aggressiveness: endometroid (e.g., TOV112D cell line); serous (e.g., OV90, SKOV3 cell lines); and clear cell (e.g., ES2 cell line) cell types. The percentage of CD44+/CD24− in particular populations of cancer cell lines was determined using FACS analysis and is shown in Table 1.

TABLE 1

| | Population |
|---|---|
| | CD44+/CD24− |
| Cancer cell line | (%) |
| TOV112D | 0.5 |
| SKOV3 | 66 |
| OV90 | 77 |
| ES2 | 99 |

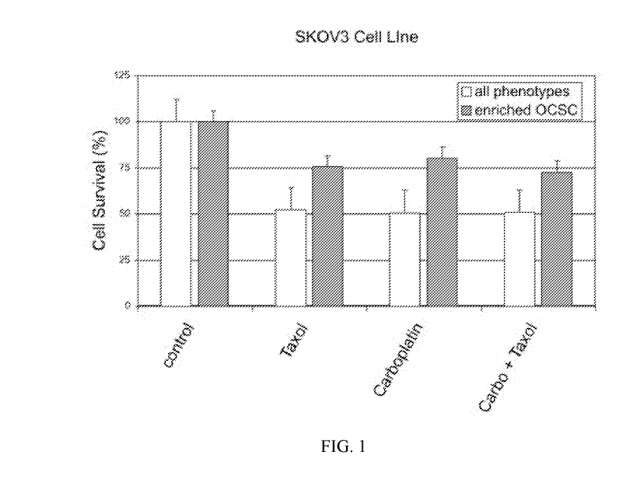

The increased resistance of ovarian cancer stem cells (SKOV3) compared to cells with other ovarian cancer cells is illustrated in FIG. 1. Particular genes are upregulated in ovarian cancer stem cells compared to non ovarian cancer stem cells (Table 2). All genes listed in Table 2, except IL-8, THBS1, and SNCG have been linked to the Hedgehog pathway.

TABLE 2

| | | Fold |
|---|---|---|
| Function | Gene | upregulation |
| Stem cell | ETS2 | 6.21 |
| development | | |
| Anti-apoptosis | BCL2L1 | 2.54 |
| | CFLAR | 2.63 |
| | MDM2 | 3.41 |
| Angiogenesis | ANGPT2 | 6.06 |
| | MET | 9.40 |
| | PIK3R1 | 3.44 |
| | IL-8 | 2.69 |
| | THBS1 | 6.09 |
| Invasion and | PLAU | 2.97 |
| metastasis | PLAUR | 5.83 |
| | SNCG | 5.83 |
| | MMP1 | 2.34 |
| Adhesion | ITGA1 | 3.68 |

### BMS-833923 (XL139)

BMS-833923 (N-{2-methyl-5 [(methylamino)methyl]phenyl}-4-[(4-phenylquinazolin-2-yl)amino]benzamide, and also known as XL139), is a small molecule inhibitor of Smoothened (SMO), a component of the hedgehog (Hh) signaling pathway (See e.g., U.S. Pat. No. 8,222,263; Siu L. et al., J. Clin. Oncol. 2010; 28:15s (suppl; abstr 2501); and National Institute of Health Clinical Trial Identifier No. NCT006701891, NCT00909402, NCT00927875, NCT01218477, NCT01357655 and NCT01413906), the disclosures of which are incorporated herein by reference in their entireties.

The structure of BMS-833923 is:

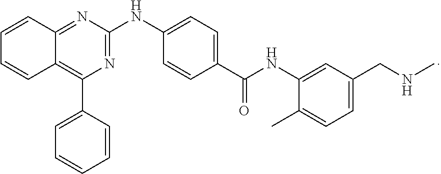 [SMILES: CNCc1ccc(C)c(NC(=O)c2ccc(Nc3nc(-c4ccccc4)c4ccccc4n3)cc2)c1]

The Hh signaling pathway plays a critical role in cell differentiation and proliferation. Dysregulation of this pathway contributes to uncontrolled growth of some tumors and resistance to chemotherapy treatments. The Hh pathway may be a regulator of CSCs, which are discrete tumor cell populations that display self-renewal and tumorigenic properties. A number of human cancers are associated with mutations in the Hh pathway, overexpression of pathway components, or CSCs with Hh pathway activation. Signaling through the Hh pathway involves Smoothened (SMO), a receptor associated with initiation of growth signals, and Patched 1, a receptor that inhibits signaling by SMO.

### Hedgehog Pathway Inhibitors

Inhibitors of the hedgehog pathway are listed in Table 3 and disclosed in U.S. Pat. No. 8,222,263, the disclosure of which is incorporated herein by reference in its entirety. In some embodiments of the methods, compositions and kits provided herein hedgehog pathway inhibitors, including SMO inhibitors, include compounds of Formula I:

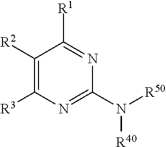

or a single isomer thereof; where the compound is optionally as a pharmaceutically acceptable salt, hydrate, solvate or combination thereof, wherein
R¹ is alkyl, cycloalkyl, phenyl, heteroaryl, or heterocycloalkyl where the cycloalkyl, phenyl, heteroaryl, and heterocycloalkyl are optionally substituted with 1, 2, or 3 R⁶;
R² and R³ together with the pyrimidinyl to which they are attached form a quinazolinyl optionally substituted at the 5-, 6-, 7-, and 8-positions with one or two groups independently selected from alkyl, alkoxy, halo, hydroxy, heterocycloalkylalkyloxy, heterocycloalkyl, and heterocycloalkyl substituted with alkyl; or
R² and R³ together with the pyrimidinyl to which they are attached form a pyrido[3,2-d]pyrimidinyl, pyrido[4,3-d]pyrimidinyl, pyrido[3,4-d]pyrimidinyl, or pyrido[2,3-d]pyrimidinyl, each of which is optionally substituted at a carbon atom at the 5-, 6-, 7-, and 8-positions with one or two groups independently selected from alkyl, alkoxy, halo, hydroxy, heterocycloalkylalkyloxy, heterocycloalkyl, and heterocycloalkyl substituted with alkyl; or
R² and R³ together with the pyrimidinyl to which they are attached form a 6,7-dihydro-5H-cyclopenta[d]pyrimidinyl, 5,6,7,8-tetrahydroquinazolinyl, or 6,7,8,9-tetrahydro-5H-cyclohepta[d]pyrimidinyl; or
R² and R³ together with the pyrimidinyl to which they are attached form a 5,6,7,8-tetrahydropyrido[3,2-d]pyrimidinyl, 5,6,7,8-tetrahydropyrido[4,3-d]pyrimidinyl, 5,6,7,8-tetrahydropyrido[3,4-d]pyrimidinyl, or 5,6,7,8-tetrahydropyrido[2,3-d]pyrimidinyl, each of which is optionally substituted at the 5-, 6-, 7-, and 8-positions with one or two groups independently selected from alkyl, alkoxycarbonyl, benzyloxycarbonyl, and optionally substituted phenylalkyl;
each R⁶, when R⁶ is present, is independently selected from alkyl, alkoxy, amino, alkylamino, dialkylamino, halo, haloalkyl, haloalkoxy, halophenyl, aminocarbonyl, alkylaminocarbonyl, dialkylaminocarbonyl, hydroxyalkyl, alkoxycarbonyl, aminoalkyl, alkylaminoalkyl, dialkylaminoalkyl, amino alkylamino, alkylaminoalkylamino, dialkylaminoalkylamino, alkyloxyalkylamino, heterocycloalkyl, and heterocycloalkylalkyl where the heterocycloalkyl, either alone or as part of heterocycloalkylalkyl, is optionally substituted with alkyl or alkoxycarbonyl;
R⁴⁰ is hydrogen or alkyl;
R⁵⁰ is selected from

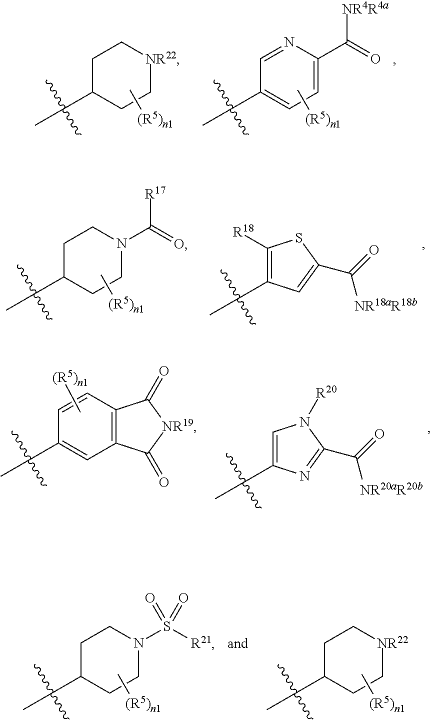

n1 is 0, 1, or 2;
each R⁵, when R⁵ is present, is independently alkyl, hydroxy, alkoxy, amino, alkylamino, dialkylamino, halo, nitro, heterocycloalkyl, heterocycloalkylamino, or heterocycloalkylalkyloxy; where each heterocycloalkyl, either alone or as part of another group in R⁵, is independently optionally substituted with alkyl or alkoxycarbonyl;
R^{4a} is hydrogen or alkyl;
R⁴ is heteroaryl substituted with one R⁸ and additionally substituted with or 2 R^{8a}; R⁴ is phenyl substituted with one R²⁹ and additionally substituted with 1 or 2 R^{9a}; R⁴ is cycloalkyl optionally substituted with one or two groups independently selected from alkyl, hydroxy, alkoxy, amino, alkylamino, and dialkylamino; or R⁴ is heterocycloalkyl optionally substituted with alkyl or alkoxycarbonyl;
R¹⁷ is cycloalkyl, heterocycloalkyl (optionally substituted with one or two groups selected from alkyl and alkoxycarbonyl), phenylalkylamino, phenylalkyl, or phenyl; and where each phenyl, either alone or as part of a group in R¹⁷, is substituted with 1, 2, or 3 R^{9a};
R¹⁸ is hydrogen, halo, or alkyl;
R^{18a} is hydrogen or alkyl;
R^{18b} is heteroaryl substituted with 1, 2, or 3 R^{8a} or R^{18b} is phenyl substituted with 1, 2, or 3 R^{9a};
R¹⁹ is phenyl substituted with 1, 2, or 3 R^{9a} or R¹⁹ is heteroaryl substituted with 1, 2, or 3 R^{8a};
R²⁰ is hydrogen, alkyl, alkylcarbonyl, alkylsulfonyl, or alkoxycarbonyl;
R^{20a} is hydrogen or alkyl;
R^{20b} is heteroaryl substituted with 1, 2, or 3 R^{8a} or R^{20b} is phenyl substituted with 1, 2, or 3 R^{9a}
R²¹ is phenyl substituted with 1, 2, or 3 R^{9a}; or R²¹ is heteroaryl substituted with 1, 2, or 3 R^{8a}; or R²¹ is heterocycloalkyl optionally substituted with alkyl or alkoxycarbonyl;
R²² is phenyl substituted with 1, 2, or 3 R^{9a} or R²² is heteroaryl substituted with 1, 2, or 3 R^{9b};
each R⁸ is independently alkyl, cycloalkyl, phenylalkyloxyalkyl, or R^{9b};
each R^{8a} is independently hydrogen, halo, or R⁸;
each R^{9a} is independently hydrogen, R^{9b}, or R^{9c};
R²⁹ is R^{9b} or R^{9c}; provided that R²⁹ is R^{9b} when R¹ is heterocycloalkyl, when R¹ is unsubstituted phenyl, and when R¹ is phenyl substituted with 1, 2, or 3 R⁶ independently selected from alkyl, halo, alkoxy, hydroxyalkyl, aminoalkyl, and alkoxycarbonyl;
each R^{9b}, when R^{9b} is present, is independently cyano, alkyl substituted with one or two R¹¹; amino; alkylamino; dialkylamino; optionally substituted heterocycloalkyl; optionally substituted heterocycloalkylalkyloxy; aminoalkyloxy; alkylaminoalkyloxy; dialkylaminoalkyloxy; optionally substituted heteroaryl; cyano; —C(O)R¹⁴; —CR^{14a} (═NR^{14b}); —C(═NR²⁴)R^{24a}; —S(O)₂NR¹³R^{13a}; —NR²³C(O)R^{23a} or—C(O)NR¹²R^{12a};
each R^{9c}, when R^{9c} is present, is independently alkyl, haloalkyl, hydroxyalkyl, halo, hydroxy, alkoxy, cyano, nitro, or phenylcarbonyl;
each R¹¹ is independently selected from hydroxy, —NR¹⁵R^{15a}, optionally substituted heteroaryl, optionally substituted heterocycloalkyl, and optionally substituted cycloalkyl;
R¹² is hydrogen or alkyl; and R^{12a} is hydrogen, hydroxy, alkoxy, alkyl, aminoalkyl, alkylaminoalkyl, dialkylaminoalkyl, hydroxyalkyl, optionally substituted heterocycloalkyl, optionally substituted heterocycloalkylalkyl, or optionally substituted heteroaryl; or R¹² and R^{12a} together with the nitrogen to which they are attached form a heterocycloalkyl optionally substituted with 1, 2, or 3 groups independently selected from alkyl, hydroxyalkyl, haloalkyl, alkylcarbonyl, alkoxycarbonyl, optionally substituted cycloalkyl, optionally substituted cycloalkylalkyl, optionally substituted heteroaryl, optionally substituted heteroarylalkyl, optionally substituted phenyl, and optionally substituted phenylalkyl;
R¹³ is hydrogen or alkyl;
R^{13a} is alkyl, aminoalkyl, alkylaminoalkyl, or dialkylaminoalkyl;
each R¹⁴ is independently hydrogen, alkyl, hydroxy, alkoxy, optionally substituted heteroarylalkyl, or optionally substituted heterocycloalkylalkyl;
each R^{14a} is hydrogen or alkyl; and R^{14b} is alkoxy, amino, alkylamino, dialkylamino, or optionally substituted heterocycloalkyl;
R¹⁵ is hydrogen, alkyl, alkoxyalkyl, hydroxyalkyl, or haloalkyl;
R^{15a} is hydrogen, alkyl, alkoxyalkyl, haloalkyl, hydroxyalkyl, carboxyalkyl, aminocarbonylalkyl, alkylaminocarbonylalkyl, dialkylaminocarbonylalkyl, optionally substituted cycloalkyl, or optionally substituted phenylalkyl;
R²³ is hydrogen or alkyl;
R^{23a} is hydrogen, alkyl, aminoalkyl, alkylaminoalkyl, dialkylaminoalkyl, or optionally substituted heterocycloalkylalkyl; and
R²⁴ is hydrogen or alkyl, hydroxy, or alkoxy; R^{24a} is hydroxy, alkoxy, amino, alkylamino, or dialkylamino.

In some embodiments of the methods, compositions and kits provided herein hedgehog pathway inhibitors, including SMO inhibitors, include compounds of Table 3.

TABLE 3
Example compounds of Formula I

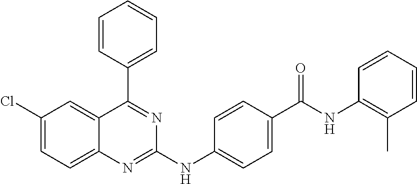 [SMILES: Cc1ccccc1NC(=O)c1ccc(Nc2nc(-c3ccccc3)c3cc(Cl)ccc3n2)cc1]

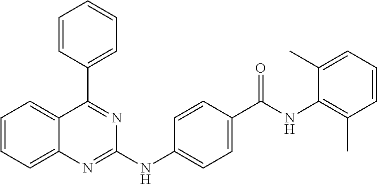 [SMILES: Cc1cccc(C)c1NC(=O)c1ccc(Nc2nc(-c3ccccc3)c3ccccc3n2)cc1]

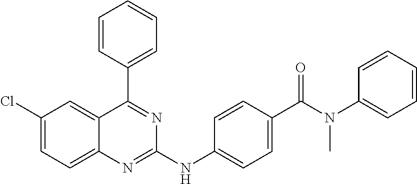 [SMILES: CN(C(=O)c1ccc(Nc2nc(-c3ccccc3)c3cc(Cl)ccc3n2)cc1)c1ccccc1]

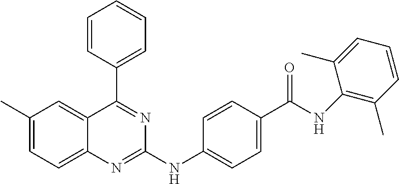 [SMILES: Cc1ccc2nc(Nc3ccc(C(=O)Nc4c(C)cccc4C)cc3)nc(-c3ccccc3)c2c1]

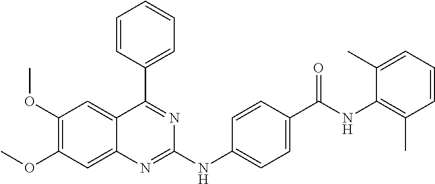 [SMILES: COc1cc2nc(Nc3ccc(C(=O)Nc4c(C)cccc4C)cc3)nc(-c3ccccc3)c2cc1OC]

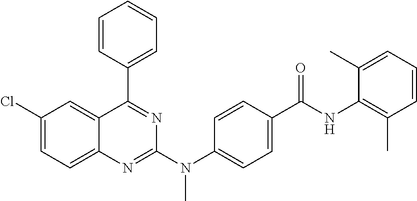 [SMILES: Cc1cccc(C)c1NC(=O)c1ccc(N(C)c2nc(-c3ccccc3)c3cc(Cl)ccc3n2)cc1]

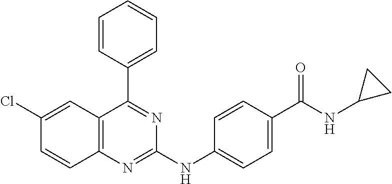 [SMILES: O=C(NC1CC1)c1ccc(Nc2nc(-c3ccccc3)c3cc(Cl)ccc3n2)cc1]

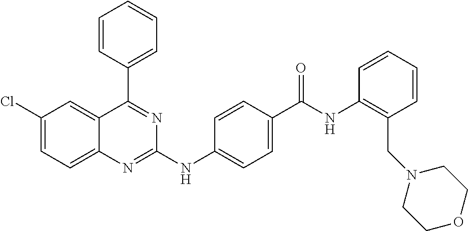 [SMILES: O=C(Nc1ccccc1CN1CCOCC1)c1ccc(Nc2nc(-c3ccccc3)c3cc(Cl)ccc3n2)cc1]

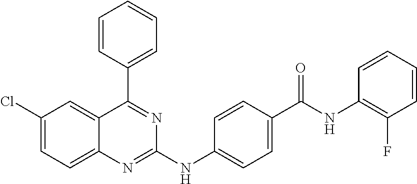 [SMILES: O=C(Nc1ccccc1F)c1ccc(Nc2nc(-c3ccccc3)c3cc(Cl)ccc3n2)cc1]

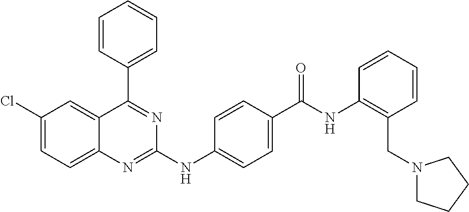 [SMILES: O=C(Nc1ccccc1CN1CCCC1)c1ccc(Nc2nc(-c3ccccc3)c3cc(Cl)ccc3n2)cc1]

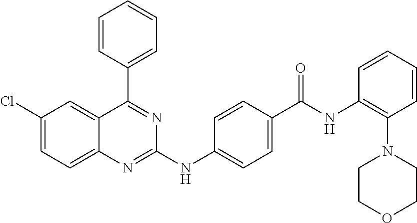 [SMILES: O=C(Nc1ccccc1N1CCOCC1)c1ccc(Nc2nc(-c3ccccc3)c3cc(Cl)ccc3n2)cc1]

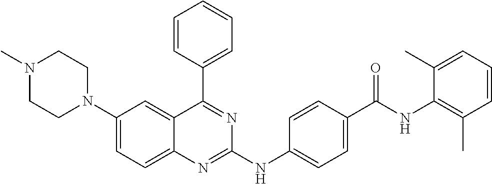 [SMILES: Cc1cccc(C)c1NC(=O)c1ccc(Nc2nc(-c3ccccc3)c3cc(N4CCN(C)CC4)ccc3n2)cc1]

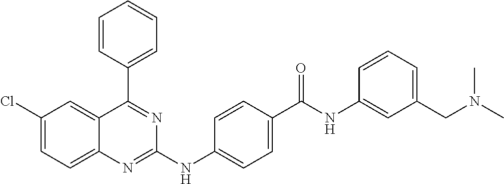 [SMILES: CN(C)Cc1cccc(NC(=O)c2ccc(Nc3nc(-c4ccccc4)c4cc(Cl)ccc4n3)cc2)c1]

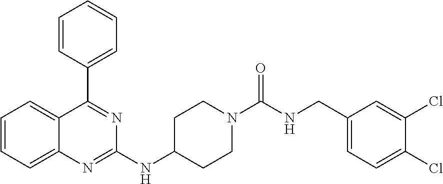 [SMILES: O=C(NCc1ccc(Cl)c(Cl)c1)N1CCC(Nc2nc(-c3ccccc3)c3ccccc3n2)CC1]

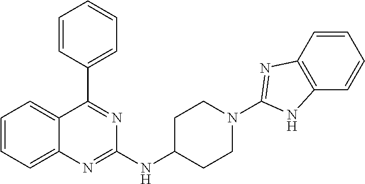 [SMILES: c1ccc(-c2nc(NC3CCN(c4nc5ccccc5[nH]4)CC3)nc3ccccc23)cc1]

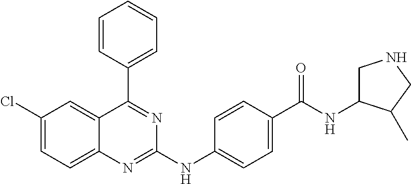 [SMILES: CC1CNCC1NC(=O)c1ccc(Nc2nc(-c3ccccc3)c3cc(Cl)ccc3n2)cc1]

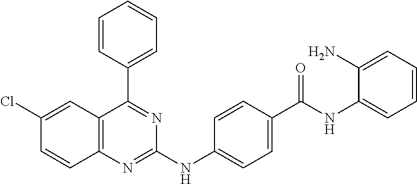 [SMILES: Nc1ccccc1NC(=O)c1ccc(Nc2nc(-c3ccccc3)c3cc(Cl)ccc3n2)cc1]

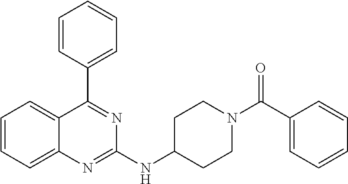 [SMILES: O=C(c1ccccc1)N1CCC(Nc2nc(-c3ccccc3)c3ccccc3n2)CC1]

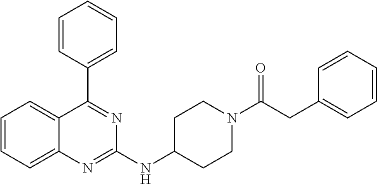 [SMILES: O=C(Cc1ccccc1)N1CCC(Nc2nc(-c3ccccc3)c3ccccc3n2)CC1]

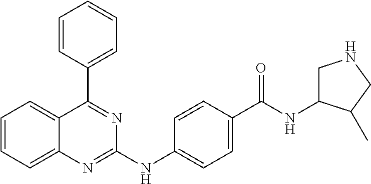 [SMILES: CC1CNCC1NC(=O)c1ccc(Nc2nc(-c3ccccc3)c3ccccc3n2)cc1]

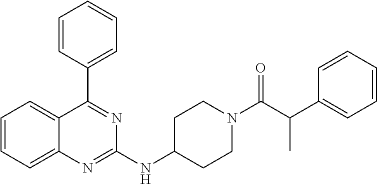 [SMILES: CC(C(=O)N1CCC(Nc2nc(-c3ccccc3)c3ccccc3n2)CC1)c1ccccc1]

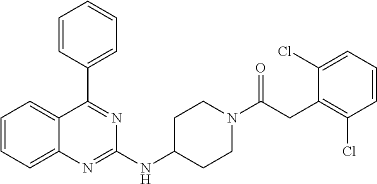 [SMILES: O=C(Cc1c(Cl)cccc1Cl)N1CCC(Nc2nc(-c3ccccc3)c3ccccc3n2)CC1]

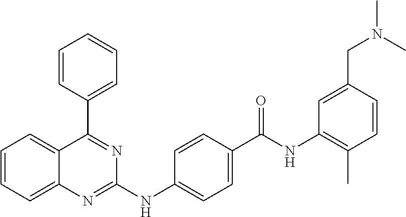 [SMILES: Cc1ccc(CN(C)C)cc1NC(=O)c1ccc(Nc2nc(-c3ccccc3)c3ccccc3n2)cc1]

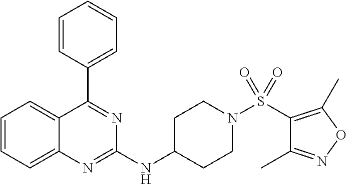 [SMILES: Cc1noc(C)c1S(=O)(=O)N1CCC(Nc2nc(-c3ccccc3)c3ccccc3n2)CC1]

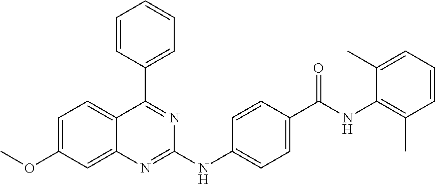 [SMILES: COc1ccc2c(-c3ccccc3)nc(Nc3ccc(C(=O)Nc4c(C)cccc4C)cc3)nc2c1]

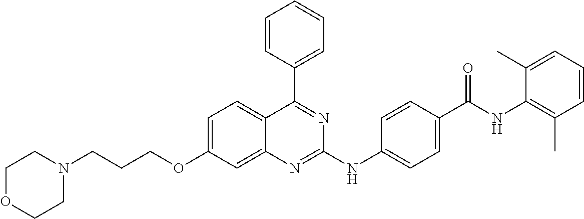 [SMILES: Cc1cccc(C)c1NC(=O)c1ccc(Nc2nc(-c3ccccc3)c3ccc(OCCCN4CCOCC4)cc3n2)cc1]

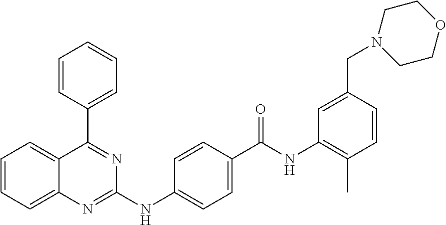 [SMILES: Cc1ccc(CN2CCOCC2)cc1NC(=O)c1ccc(Nc2nc(-c3ccccc3)c3ccccc3n2)cc1]

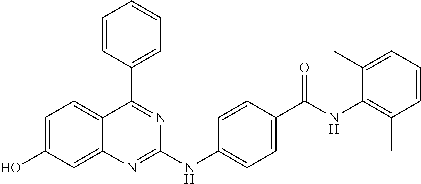 [SMILES: Cc1cccc(C)c1NC(=O)c1ccc(Nc2nc(-c3ccccc3)c3ccc(O)cc3n2)cc1]

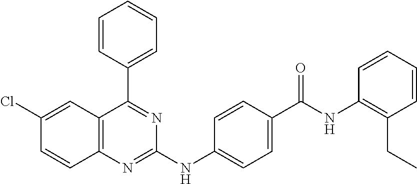 [SMILES: CCc1ccccc1NC(=O)c1ccc(Nc2nc(-c3ccccc3)c3cc(Cl)ccc3n2)cc1]

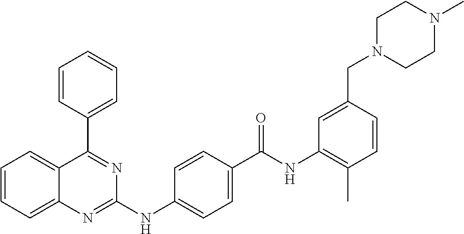 [SMILES: Cc1ccc(CN2CCN(C)CC2)cc1NC(=O)c1ccc(Nc2nc(-c3ccccc3)c3ccccc3n2)cc1]

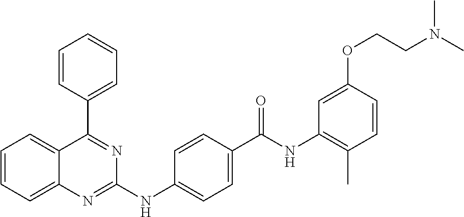 [SMILES: Cc1ccc(OCCN(C)C)cc1NC(=O)c1ccc(Nc2nc(-c3ccccc3)c3ccccc3n2)cc1]

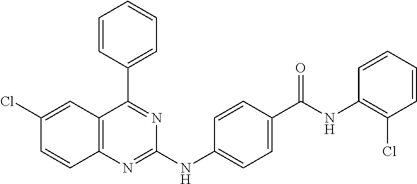 [SMILES: O=C(Nc1ccccc1Cl)c1ccc(Nc2nc(-c3ccccc3)c3cc(Cl)ccc3n2)cc1]

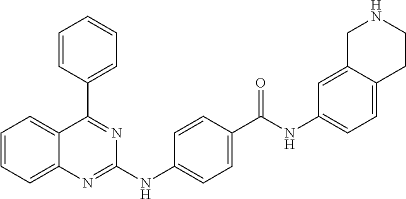 [SMILES: O=C(Nc1ccc2c(c1)CNCC2)c1ccc(Nc2nc(-c3ccccc3)c3ccccc3n2)cc1]

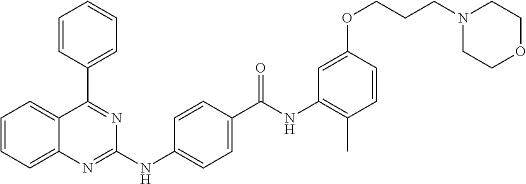 [SMILES: Cc1ccc(OCCCN2CCOCC2)cc1NC(=O)c1ccc(Nc2nc(-c3ccccc3)c3ccccc3n2)cc1]

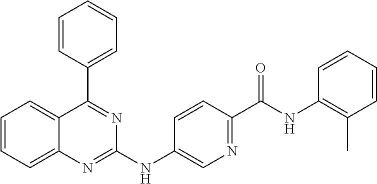 [SMILES: Cc1ccccc1NC(=O)c1ccc(Nc2nc(-c3ccccc3)c3ccccc3n2)cn1]

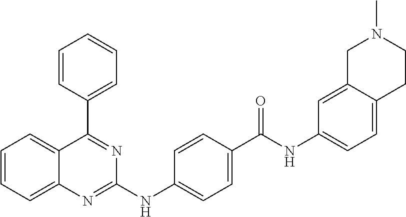 [SMILES: CN1CCc2ccc(NC(=O)c3ccc(Nc4nc(-c5ccccc5)c5ccccc5n4)cc3)cc2C1]

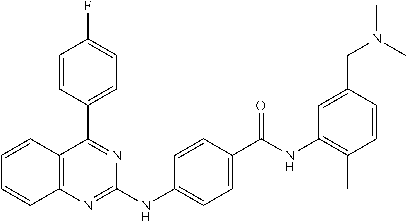 [SMILES: Cc1ccc(CN(C)C)cc1NC(=O)c1ccc(Nc2nc(-c3ccc(F)cc3)c3ccccc3n2)cc1]

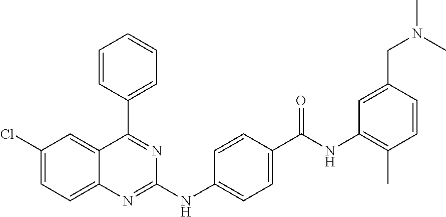 [SMILES: Cc1ccc(CN(C)C)cc1NC(=O)c1ccc(Nc2nc(-c3ccccc3)c3cc(Cl)ccc3n2)cc1]

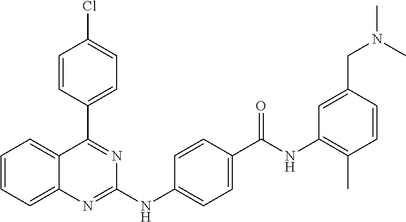 [SMILES: Cc1ccc(CN(C)C)cc1NC(=O)c1ccc(Nc2nc(-c3ccc(Cl)cc3)c3ccccc3n2)cc1]

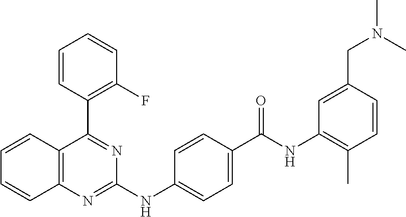 [SMILES: Cc1ccc(CN(C)C)cc1NC(=O)c1ccc(Nc2nc(-c3ccccc3F)c3ccccc3n2)cc1]

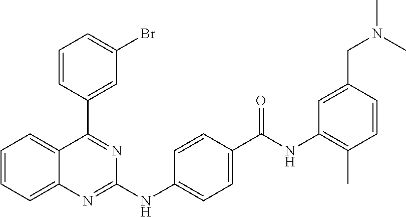 [SMILES: Cc1ccc(CN(C)C)cc1NC(=O)c1ccc(Nc2nc(-c3cccc(Br)c3)c3ccccc3n2)cc1]

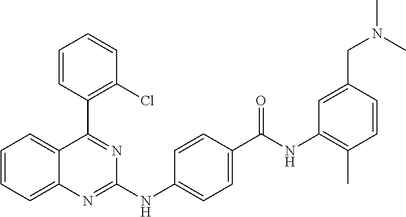 [SMILES: Cc1ccc(CN(C)C)cc1NC(=O)c1ccc(Nc2nc(-c3ccccc3Cl)c3ccccc3n2)cc1]

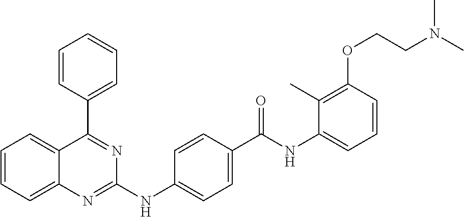 [SMILES: Cc1c(NC(=O)c2ccc(Nc3nc(-c4ccccc4)c4ccccc4n3)cc2)cccc1OCCN(C)C]

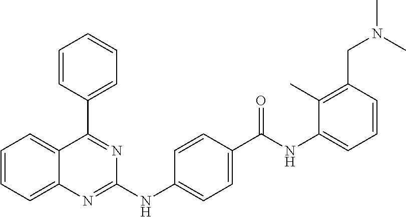 [SMILES: Cc1c(CN(C)C)cccc1NC(=O)c1ccc(Nc2nc(-c3ccccc3)c3ccccc3n2)cc1]

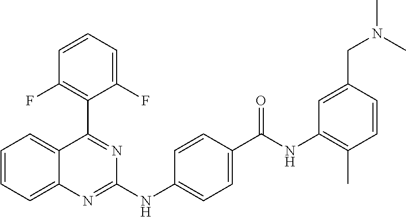 [SMILES: Cc1ccc(CN(C)C)cc1NC(=O)c1ccc(Nc2nc(-c3c(F)cccc3F)c3ccccc3n2)cc1]

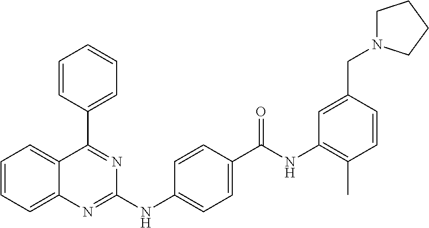 [SMILES: Cc1ccc(CN2CCCC2)cc1NC(=O)c1ccc(Nc2nc(-c3ccccc3)c3ccccc3n2)cc1]

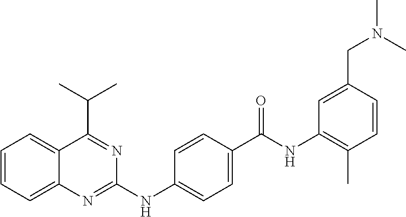 [SMILES: Cc1ccc(CN(C)C)cc1NC(=O)c1ccc(Nc2nc(C(C)C)c3ccccc3n2)cc1]

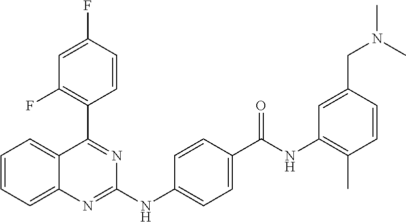 [SMILES: Cc1ccc(CN(C)C)cc1NC(=O)c1ccc(Nc2nc(-c3ccc(F)cc3F)c3ccccc3n2)cc1]

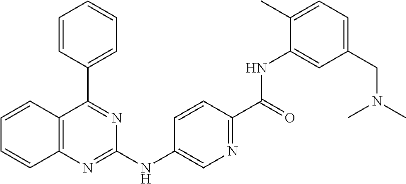 [SMILES: Cc1ccc(CN(C)C)cc1NC(=O)c1ccc(Nc2nc(-c3ccccc3)c3ccccc3n2)cn1]

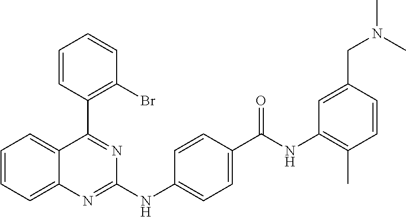 [SMILES: Cc1ccc(CN(C)C)cc1NC(=O)c1ccc(Nc2nc(-c3ccccc3Br)c3ccccc3n2)cc1]

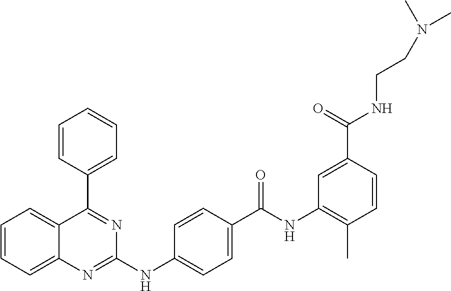 [SMILES: Cc1ccc(C(=O)NCCN(C)C)cc1NC(=O)c1ccc(Nc2nc(-c3ccccc3)c3ccccc3n2)cc1]

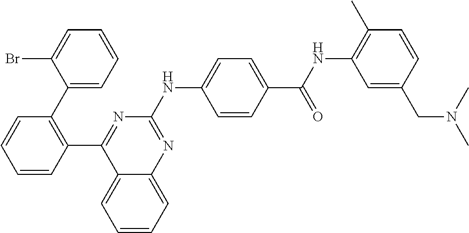 [SMILES: Cc1ccc(CN(C)C)cc1NC(=O)c1ccc(Nc2nc(-c3ccccc3-c3ccccc3Br)c3ccccc3n2)cc1]

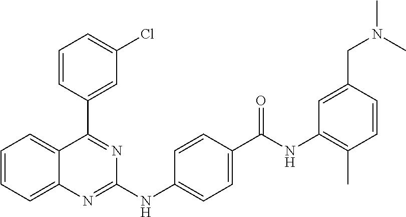 [SMILES: Cc1ccc(CN(C)C)cc1NC(=O)c1ccc(Nc2nc(-c3cccc(Cl)c3)c3ccccc3n2)cc1]

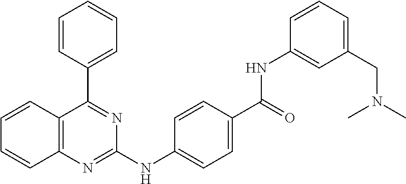 [SMILES: CN(C)Cc1cccc(NC(=O)c2ccc(Nc3nc(-c4ccccc4)c4ccccc4n3)cc2)c1]

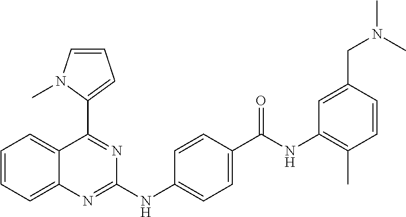 [SMILES: Cc1ccc(CN(C)C)cc1NC(=O)c1ccc(Nc2nc(-c3cccn3C)c3ccccc3n2)cc1]

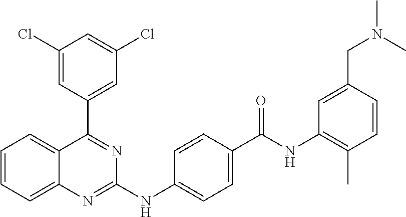 [SMILES: Cc1ccc(CN(C)C)cc1NC(=O)c1ccc(Nc2nc(-c3cc(Cl)cc(Cl)c3)c3ccccc3n2)cc1]

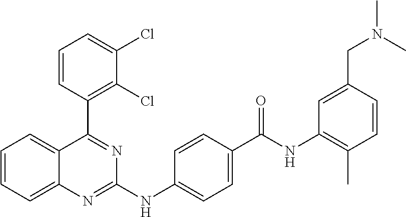 [SMILES: Cc1ccc(CN(C)C)cc1NC(=O)c1ccc(Nc2nc(-c3cccc(Cl)c3Cl)c3ccccc3n2)cc1]

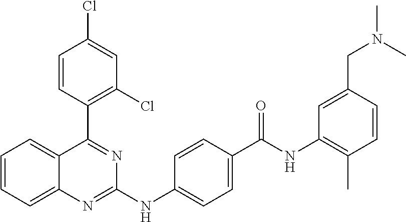 [SMILES: Cc1ccc(CN(C)C)cc1NC(=O)c1ccc(Nc2nc(-c3ccc(Cl)cc3Cl)c3ccccc3n2)cc1]

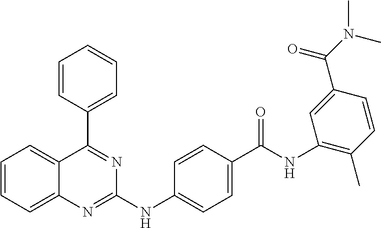 [SMILES: Cc1ccc(C(=O)N(C)C)cc1NC(=O)c1ccc(Nc2nc(-c3ccccc3)c3ccccc3n2)cc1]

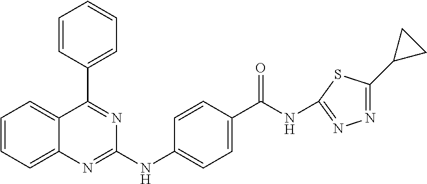 [SMILES: O=C(Nc1nnc(C2CC2)s1)c1ccc(Nc2nc(-c3ccccc3)c3ccccc3n2)cc1]

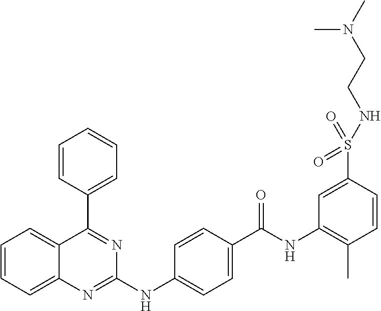 [SMILES: Cc1ccc(S(=O)(=O)NCCN(C)C)cc1NC(=O)c1ccc(Nc2nc(-c3ccccc3)c3ccccc3n2)cc1]

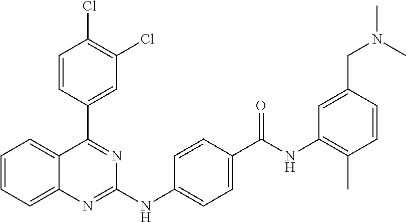 [SMILES: Cc1ccc(CN(C)C)cc1NC(=O)c1ccc(Nc2nc(-c3ccc(Cl)c(Cl)c3)c3ccccc3n2)cc1]

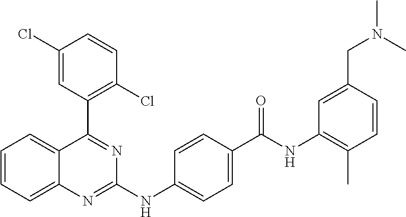 [SMILES: Cc1ccc(CN(C)C)cc1NC(=O)c1ccc(Nc2nc(-c3cc(Cl)ccc3Cl)c3ccccc3n2)cc1]

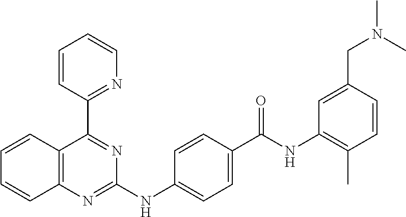 [SMILES: Cc1ccc(CN(C)C)cc1NC(=O)c1ccc(Nc2nc(-c3ccccn3)c3ccccc3n2)cc1]

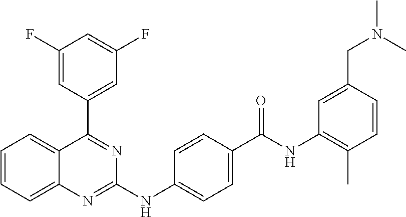 [SMILES: Cc1ccc(CN(C)C)cc1NC(=O)c1ccc(Nc2nc(-c3cc(F)cc(F)c3)c3ccccc3n2)cc1]

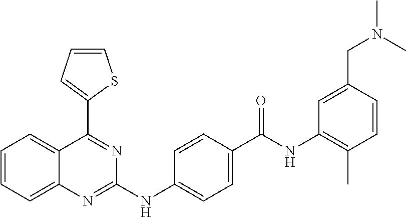 [SMILES: Cc1ccc(CN(C)C)cc1NC(=O)c1ccc(Nc2nc(-c3cccs3)c3ccccc3n2)cc1]

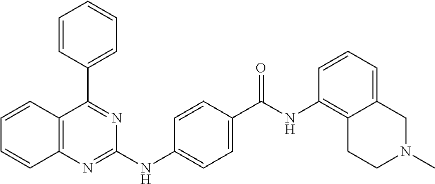 [SMILES: CN1CCc2c(cccc2NC(=O)c2ccc(Nc3nc(-c4ccccc4)c4ccccc4n3)cc2)C1]

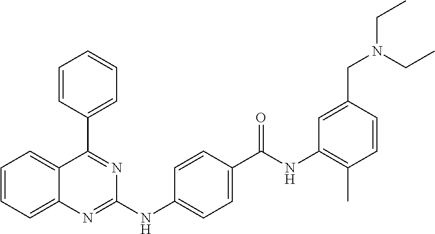 [SMILES: CCN(CC)Cc1ccc(C)c(NC(=O)c2ccc(Nc3nc(-c4ccccc4)c4ccccc4n3)cc2)c1]

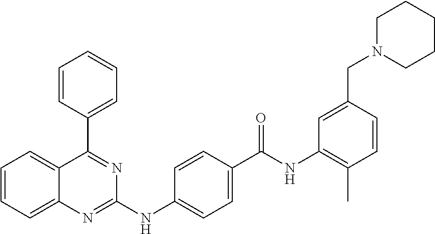 [SMILES: Cc1ccc(CN2CCCCC2)cc1NC(=O)c1ccc(Nc2nc(-c3ccccc3)c3ccccc3n2)cc1]

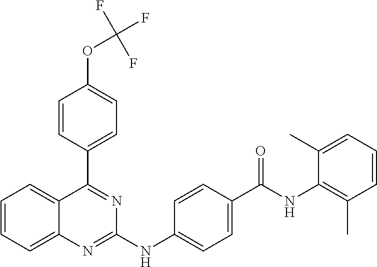 [SMILES: Cc1cccc(C)c1NC(=O)c1ccc(Nc2nc(-c3ccc(OC(F)(F)F)cc3)c3ccccc3n2)cc1]

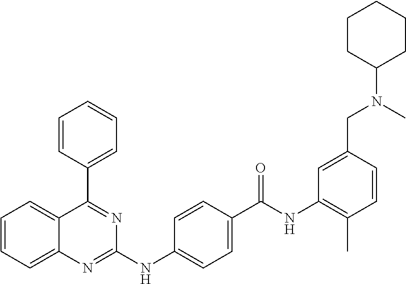 [SMILES: Cc1ccc(CN(C)C2CCCCC2)cc1NC(=O)c1ccc(Nc2nc(-c3ccccc3)c3ccccc3n2)cc1]

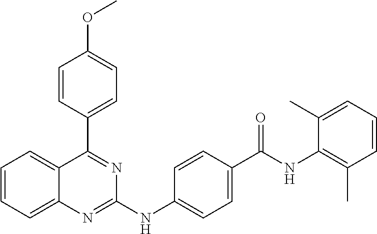 [SMILES: COc1ccc(-c2nc(Nc3ccc(C(=O)Nc4c(C)cccc4C)cc3)nc3ccccc23)cc1]

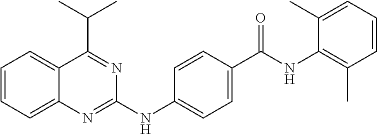 [SMILES: Cc1cccc(C)c1NC(=O)c1ccc(Nc2nc(C(C)C)c3ccccc3n2)cc1]

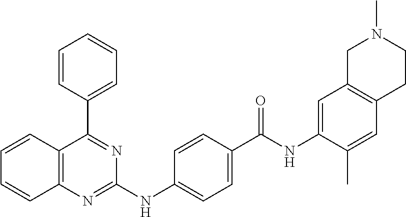 [SMILES: Cc1cc2c(cc1NC(=O)c1ccc(Nc3nc(-c4ccccc4)c4ccccc4n3)cc1)CN(C)CC2]

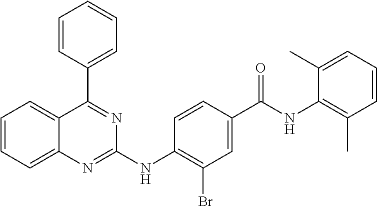 [SMILES: Cc1cccc(C)c1NC(=O)c1ccc(Nc2nc(-c3ccccc3)c3ccccc3n2)c(Br)c1]

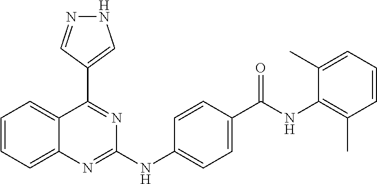 [SMILES: Cc1cccc(C)c1NC(=O)c1ccc(Nc2nc(-c3cn[nH]c3)c3ccccc3n2)cc1]

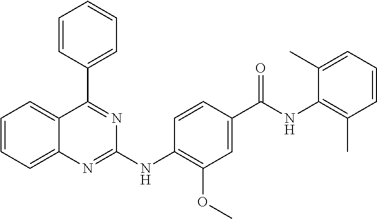 [SMILES: COc1cc(C(=O)Nc2c(C)cccc2C)ccc1Nc1nc(-c2ccccc2)c2ccccc2n1]

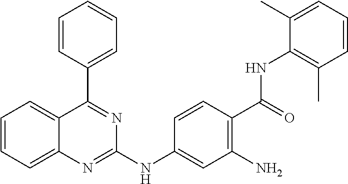 [SMILES: Cc1cccc(C)c1NC(=O)c1ccc(Nc2nc(-c3ccccc3)c3ccccc3n2)cc1N]

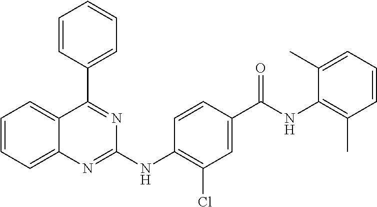 [SMILES: Cc1cccc(C)c1NC(=O)c1ccc(Nc2nc(-c3ccccc3)c3ccccc3n2)c(Cl)c1]

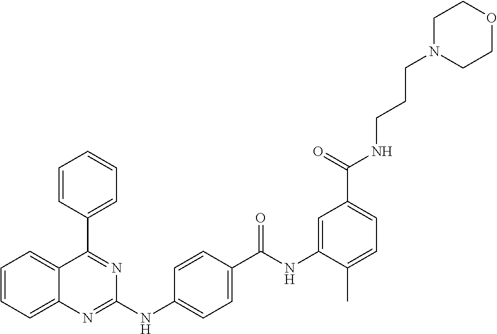 [SMILES: Cc1ccc(C(=O)NCCCN2CCOCC2)cc1NC(=O)c1ccc(Nc2nc(-c3ccccc3)c3ccccc3n2)cc1]

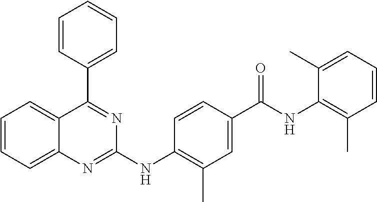 [SMILES: Cc1cc(C(=O)Nc2c(C)cccc2C)ccc1Nc1nc(-c2ccccc2)c2ccccc2n1]

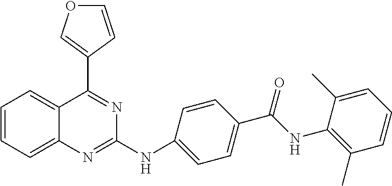 [SMILES: Cc1cccc(C)c1NC(=O)c1ccc(Nc2nc(-c3ccoc3)c3ccccc3n2)cc1]

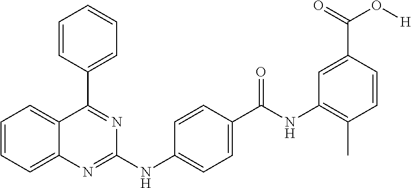 [SMILES: Cc1ccc(C(=O)O)cc1NC(=O)c1ccc(Nc2nc(-c3ccccc3)c3ccccc3n2)cc1]

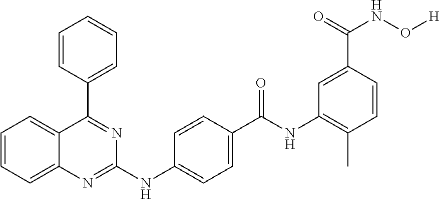 [SMILES: Cc1ccc(C(=O)NO)cc1NC(=O)c1ccc(Nc2nc(-c3ccccc3)c3ccccc3n2)cc1]

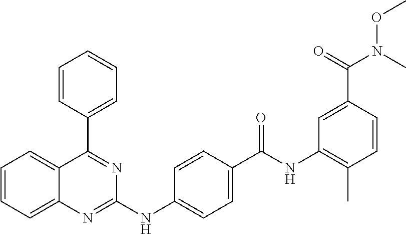 [SMILES: CON(C)C(=O)c1ccc(C)c(NC(=O)c2ccc(Nc3nc(-c4ccccc4)c4ccccc4n3)cc2)c1]

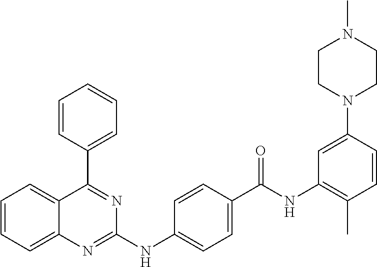 [SMILES: Cc1ccc(N2CCN(C)CC2)cc1NC(=O)c1ccc(Nc2nc(-c3ccccc3)c3ccccc3n2)cc1]

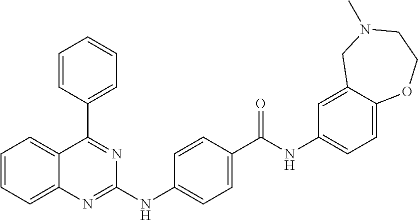 [SMILES: CN1CCOc2ccc(NC(=O)c3ccc(Nc4nc(-c5ccccc5)c5ccccc5n4)cc3)cc2C1]

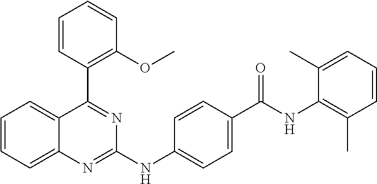 [SMILES: COc1ccccc1-c1nc(Nc2ccc(C(=O)Nc3c(C)cccc3C)cc2)nc2ccccc12]

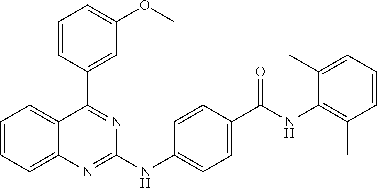 [SMILES: COc1cccc(-c2nc(Nc3ccc(C(=O)Nc4c(C)cccc4C)cc3)nc3ccccc23)c1]

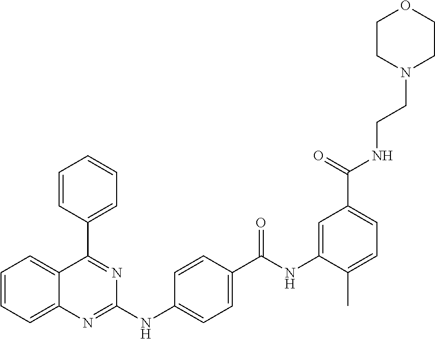 [SMILES: Cc1ccc(C(=O)NCCN2CCOCC2)cc1NC(=O)c1ccc(Nc2nc(-c3ccccc3)c3ccccc3n2)cc1]

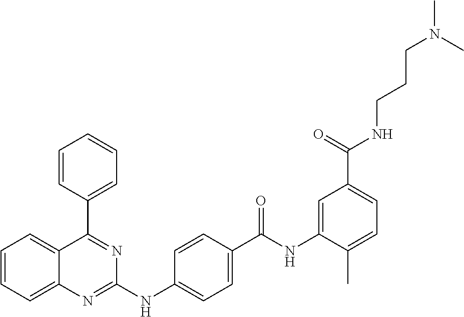 [SMILES: Cc1ccc(C(=O)NCCCN(C)C)cc1NC(=O)c1ccc(Nc2nc(-c3ccccc3)c3ccccc3n2)cc1]

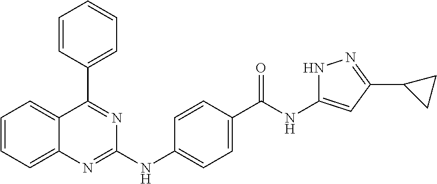 [SMILES: O=C(Nc1cc(C2CC2)n[nH]1)c1ccc(Nc2nc(-c3ccccc3)c3ccccc3n2)cc1]

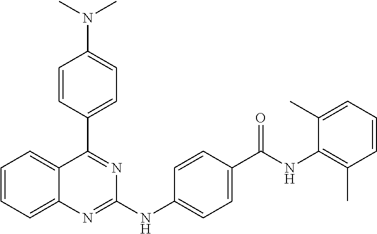 [SMILES: Cc1cccc(C)c1NC(=O)c1ccc(Nc2nc(-c3ccc(N(C)C)cc3)c3ccccc3n2)cc1]

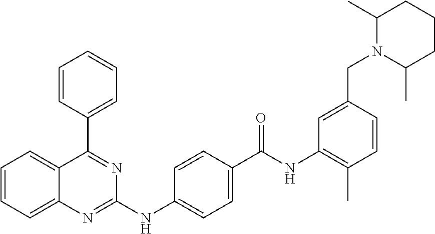 [SMILES: Cc1ccc(CN2C(C)CCCC2C)cc1NC(=O)c1ccc(Nc2nc(-c3ccccc3)c3ccccc3n2)cc1]

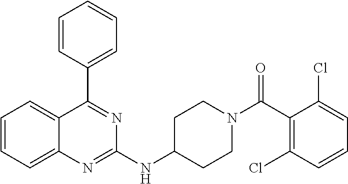 [SMILES: O=C(c1c(Cl)cccc1Cl)N1CCC(Nc2nc(-c3ccccc3)c3ccccc3n2)CC1]

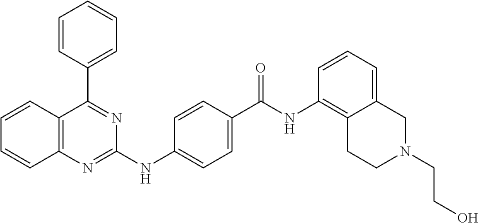 [SMILES: O=C(Nc1cccc2c1CCN(CCO)C2)c1ccc(Nc2nc(-c3ccccc3)c3ccccc3n2)cc1]

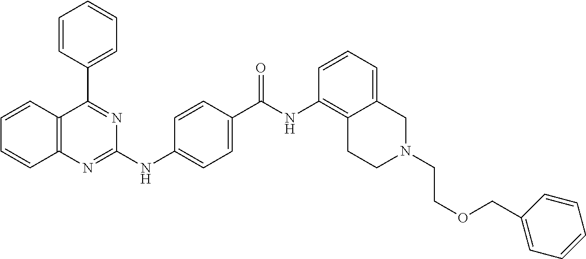 [SMILES: O=C(Nc1cccc2c1CCN(CCOCc1ccccc1)C2)c1ccc(Nc2nc(-c3ccccc3)c3ccccc3n2)cc1]

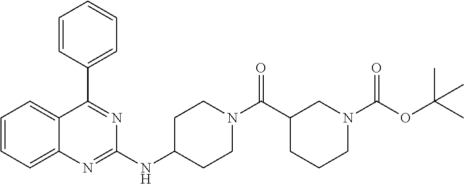 [SMILES: CC(C)(C)OC(=O)N1CCCC(C(=O)N2CCC(Nc3nc(-c4ccccc4)c4ccccc4n3)CC2)C1]

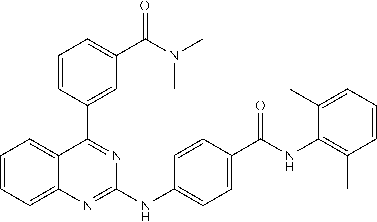 [SMILES: Cc1cccc(C)c1NC(=O)c1ccc(Nc2nc(-c3cccc(C(=O)N(C)C)c3)c3ccccc3n2)cc1]

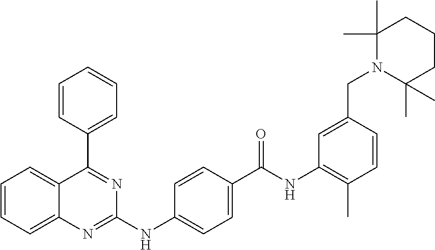 [SMILES: Cc1ccc(CN2C(C)(C)CCCC2(C)C)cc1NC(=O)c1ccc(Nc2nc(-c3ccccc3)c3ccccc3n2)cc1]

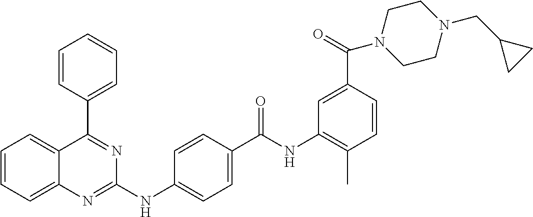 [SMILES: Cc1ccc(C(=O)N2CCN(CC3CC3)CC2)cc1NC(=O)c1ccc(Nc2nc(-c3ccccc3)c3ccccc3n2)cc1]

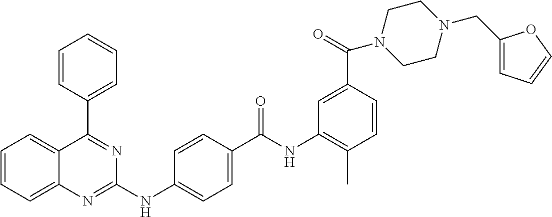 [SMILES: Cc1ccc(C(=O)N2CCN(Cc3ccco3)CC2)cc1NC(=O)c1ccc(Nc2nc(-c3ccccc3)c3ccccc3n2)cc1]

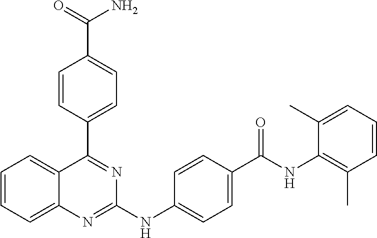 [SMILES: Cc1cccc(C)c1NC(=O)c1ccc(Nc2nc(-c3ccc(C(N)=O)cc3)c3ccccc3n2)cc1]

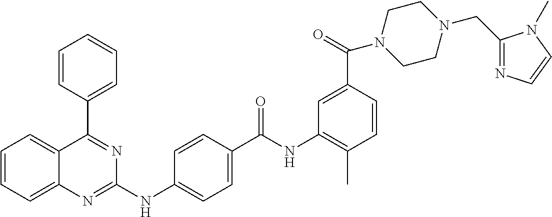 [SMILES: Cc1ccc(C(=O)N2CCN(Cc3nccn3C)CC2)cc1NC(=O)c1ccc(Nc2nc(-c3ccccc3)c3ccccc3n2)cc1]

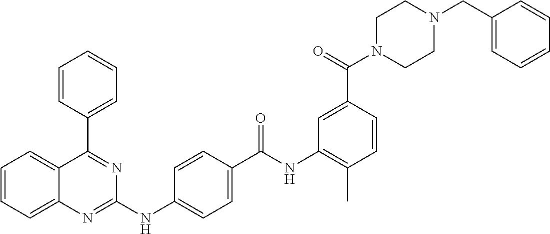 [SMILES: Cc1ccc(C(=O)N2CCN(Cc3ccccc3)CC2)cc1NC(=O)c1ccc(Nc2nc(-c3ccccc3)c3ccccc3n2)cc1]

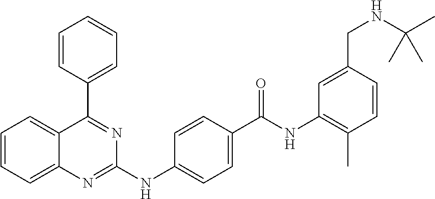 [SMILES: Cc1ccc(CNC(C)(C)C)cc1NC(=O)c1ccc(Nc2nc(-c3ccccc3)c3ccccc3n2)cc1]

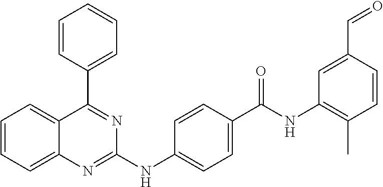 [SMILES: Cc1ccc(C=O)cc1NC(=O)c1ccc(Nc2nc(-c3ccccc3)c3ccccc3n2)cc1]

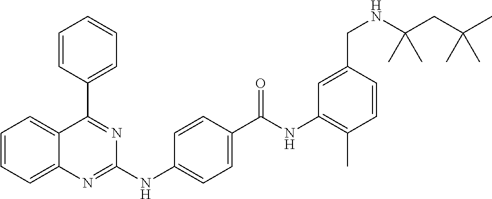 [SMILES: Cc1ccc(CNC(C)(C)CC(C)(C)C)cc1NC(=O)c1ccc(Nc2nc(-c3ccccc3)c3ccccc3n2)cc1]

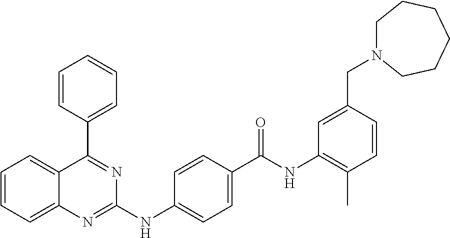 [SMILES: Cc1ccc(CN2CCCCCC2)cc1NC(=O)c1ccc(Nc2nc(-c3ccccc3)c3ccccc3n2)cc1]

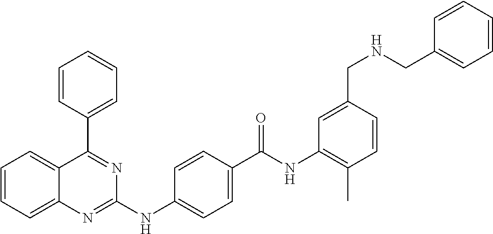 [SMILES: Cc1ccc(CNCc2ccccc2)cc1NC(=O)c1ccc(Nc2nc(-c3ccccc3)c3ccccc3n2)cc1]

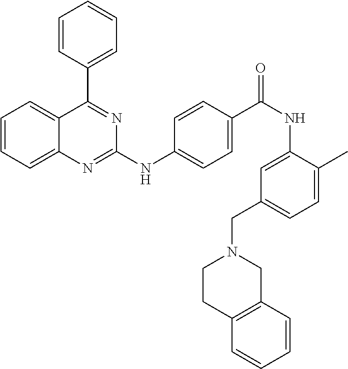 [SMILES: Cc1ccc(CN2CCc3ccccc3C2)cc1NC(=O)c1ccc(Nc2nc(-c3ccccc3)c3ccccc3n2)cc1]

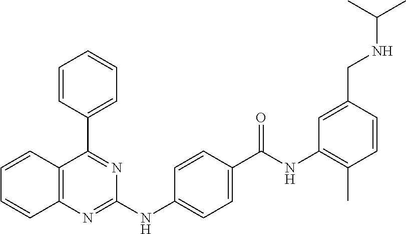 [SMILES: Cc1ccc(CNC(C)C)cc1NC(=O)c1ccc(Nc2nc(-c3ccccc3)c3ccccc3n2)cc1]

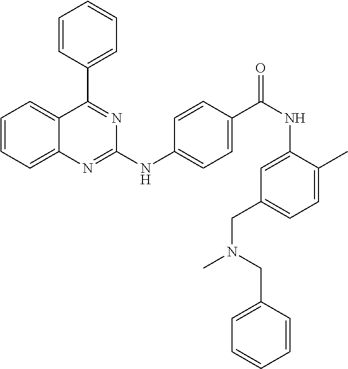 [SMILES: Cc1ccc(CN(C)Cc2ccccc2)cc1NC(=O)c1ccc(Nc2nc(-c3ccccc3)c3ccccc3n2)cc1]

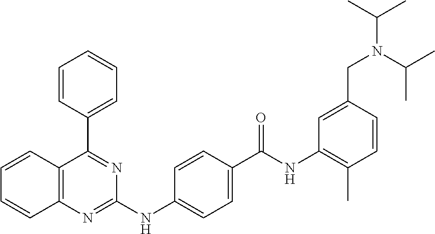 [SMILES: Cc1ccc(CN(C(C)C)C(C)C)cc1NC(=O)c1ccc(Nc2nc(-c3ccccc3)c3ccccc3n2)cc1]

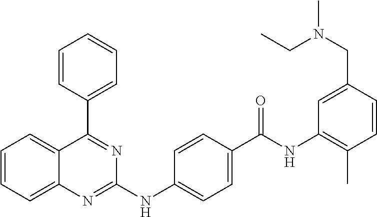 [SMILES: CCN(C)Cc1ccc(C)c(NC(=O)c2ccc(Nc3nc(-c4ccccc4)c4ccccc4n3)cc2)c1]

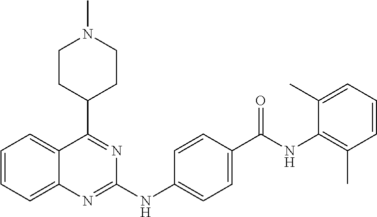 [SMILES: Cc1cccc(C)c1NC(=O)c1ccc(Nc2nc(C3CCN(C)CC3)c3ccccc3n2)cc1]

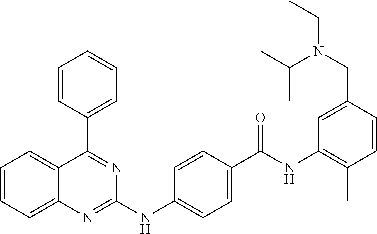 [SMILES: CCN(Cc1ccc(C)c(NC(=O)c2ccc(Nc3nc(-c4ccccc4)c4ccccc4n3)cc2)c1)C(C)C]

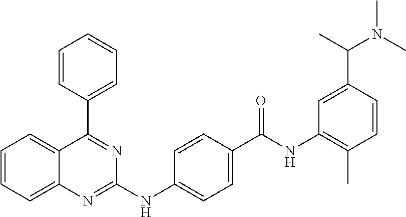 [SMILES: Cc1ccc(C(C)N(C)C)cc1NC(=O)c1ccc(Nc2nc(-c3ccccc3)c3ccccc3n2)cc1]

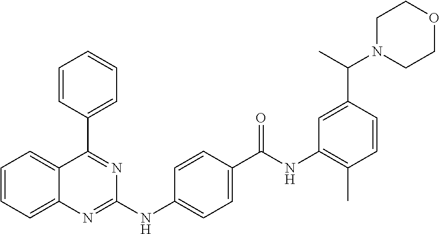 [SMILES: Cc1ccc(C(C)N2CCOCC2)cc1NC(=O)c1ccc(Nc2nc(-c3ccccc3)c3ccccc3n2)cc1]

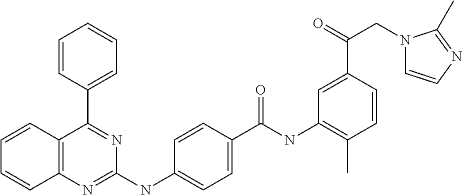 [SMILES: Cc1ccc(C(=O)Cn2ccnc2C)cc1NC(=O)c1ccc(Nc2nc(-c3ccccc3)c3ccccc3n2)cc1]

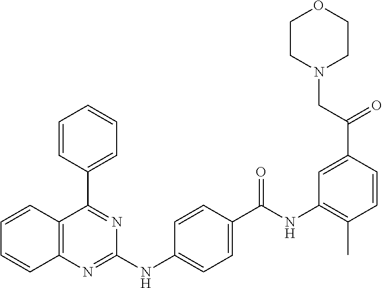 [SMILES: Cc1ccc(C(=O)CN2CCOCC2)cc1NC(=O)c1ccc(Nc2nc(-c3ccccc3)c3ccccc3n2)cc1]

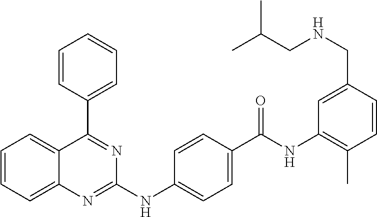 [SMILES: Cc1ccc(CNCC(C)C)cc1NC(=O)c1ccc(Nc2nc(-c3ccccc3)c3ccccc3n2)cc1]

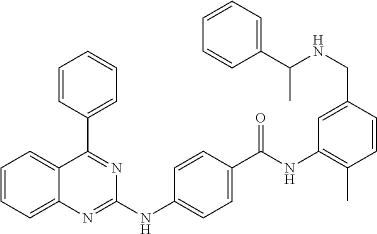 [SMILES: Cc1ccc(CNC(C)c2ccccc2)cc1NC(=O)c1ccc(Nc2nc(-c3ccccc3)c3ccccc3n2)cc1]

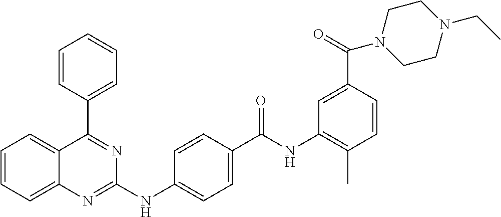 [SMILES: CCN1CCN(C(=O)c2ccc(C)c(NC(=O)c3ccc(Nc4nc(-c5ccccc5)c5ccccc5n4)cc3)c2)CC1]

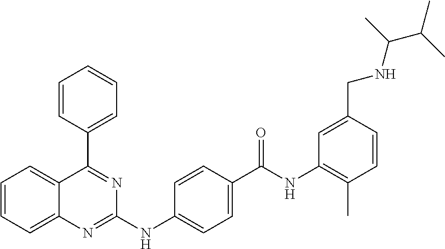 [SMILES: Cc1ccc(CNC(C)C(C)C)cc1NC(=O)c1ccc(Nc2nc(-c3ccccc3)c3ccccc3n2)cc1]

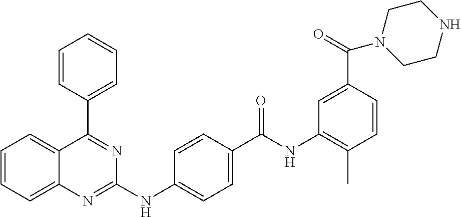 [SMILES: Cc1ccc(C(=O)N2CCNCC2)cc1NC(=O)c1ccc(Nc2nc(-c3ccccc3)c3ccccc3n2)cc1]

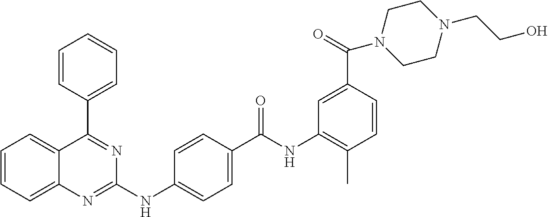 [SMILES: Cc1ccc(C(=O)N2CCN(CCO)CC2)cc1NC(=O)c1ccc(Nc2nc(-c3ccccc3)c3ccccc3n2)cc1]

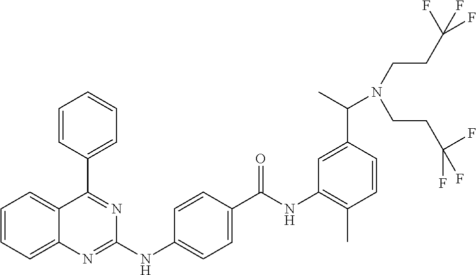 [SMILES: Cc1ccc(C(C)N(CCC(F)(F)F)CCC(F)(F)F)cc1NC(=O)c1ccc(Nc2nc(-c3ccccc3)c3ccccc3n2)cc1]

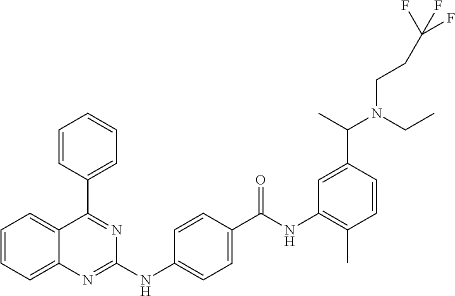 [SMILES: CCN(CCC(F)(F)F)C(C)c1ccc(C)c(NC(=O)c2ccc(Nc3nc(-c4ccccc4)c4ccccc4n3)cc2)c1]

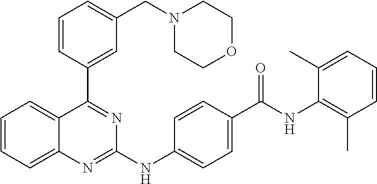 [SMILES: Cc1cccc(C)c1NC(=O)c1ccc(Nc2nc(-c3cccc(CN4CCOCC4)c3)c3ccccc3n2)cc1]

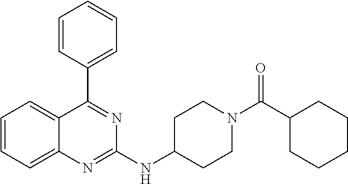 [SMILES: O=C(C1CCCCC1)N1CCC(Nc2nc(-c3ccccc3)c3ccccc3n2)CC1]

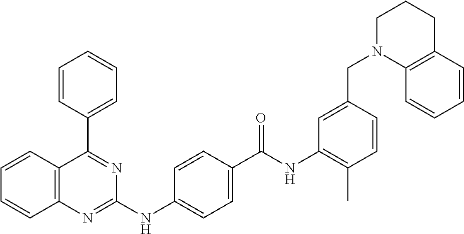 [SMILES: Cc1ccc(CN2CCCc3ccccc32)cc1NC(=O)c1ccc(Nc2nc(-c3ccccc3)c3ccccc3n2)cc1]

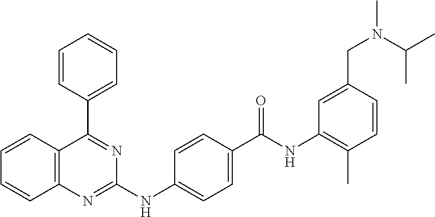 [SMILES: Cc1ccc(CN(C)C(C)C)cc1NC(=O)c1ccc(Nc2nc(-c3ccccc3)c3ccccc3n2)cc1]

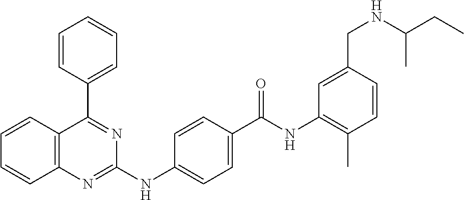 [SMILES: CCC(C)NCc1ccc(C)c(NC(=O)c2ccc(Nc3nc(-c4ccccc4)c4ccccc4n3)cc2)c1]

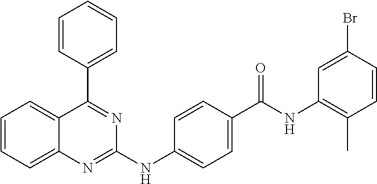 [SMILES: Cc1ccc(Br)cc1NC(=O)c1ccc(Nc2nc(-c3ccccc3)c3ccccc3n2)cc1]

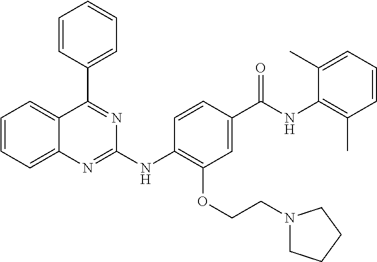 [SMILES: Cc1cccc(C)c1NC(=O)c1ccc(Nc2nc(-c3ccccc3)c3ccccc3n2)c(OCCN2CCCC2)c1]

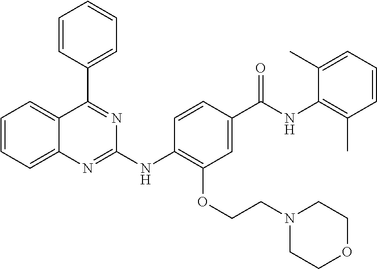 [SMILES: Cc1cccc(C)c1NC(=O)c1ccc(Nc2nc(-c3ccccc3)c3ccccc3n2)c(OCCN2CCOCC2)c1]

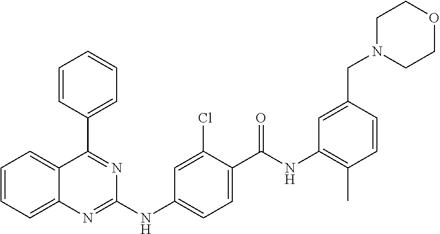 [SMILES: Cc1ccc(CN2CCOCC2)cc1NC(=O)c1ccc(Nc2nc(-c3ccccc3)c3ccccc3n2)cc1Cl]

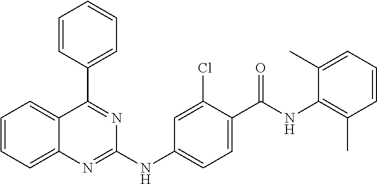 [SMILES: Cc1cccc(C)c1NC(=O)c1ccc(Nc2nc(-c3ccccc3)c3ccccc3n2)cc1Cl]

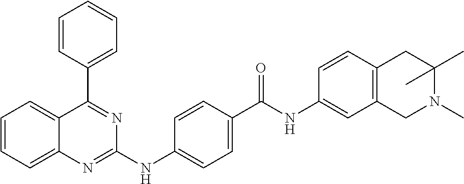

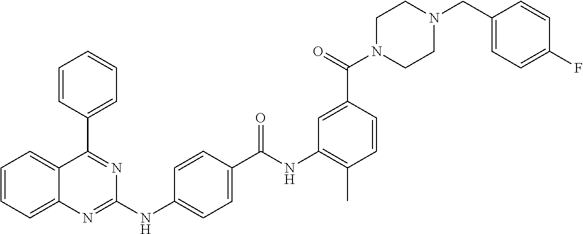 [SMILES: Cc1ccc(C(=O)N2CCN(Cc3ccc(F)cc3)CC2)cc1NC(=O)c1ccc(Nc2nc(-c3ccccc3)c3ccccc3n2)cc1]

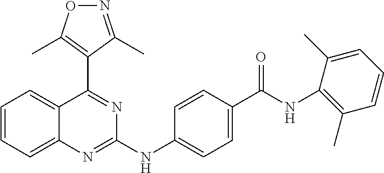 [SMILES: Cc1cccc(C)c1NC(=O)c1ccc(Nc2nc(-c3c(C)noc3C)c3ccccc3n2)cc1]

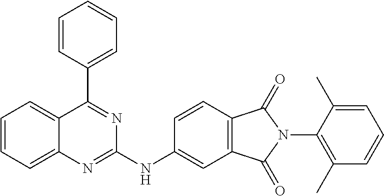 [SMILES: Cc1cccc(C)c1N1C(=O)c2ccc(Nc3nc(-c4ccccc4)c4ccccc4n3)cc2C1=O]

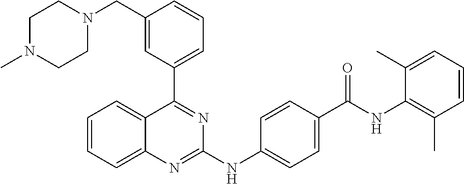 [SMILES: Cc1cccc(C)c1NC(=O)c1ccc(Nc2nc(-c3cccc(CN4CCN(C)CC4)c3)c3ccccc3n2)cc1]

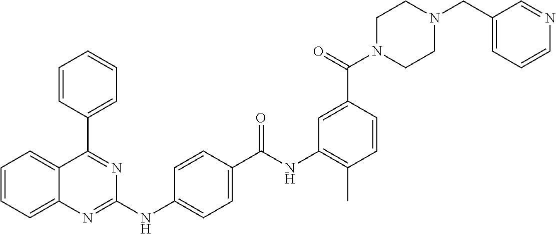 [SMILES: Cc1ccc(C(=O)N2CCN(Cc3cccnc3)CC2)cc1NC(=O)c1ccc(Nc2nc(-c3ccccc3)c3ccccc3n2)cc1]

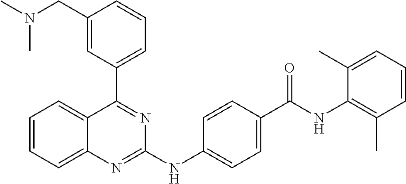 [SMILES: Cc1cccc(C)c1NC(=O)c1ccc(Nc2nc(-c3cccc(CN(C)C)c3)c3ccccc3n2)cc1]

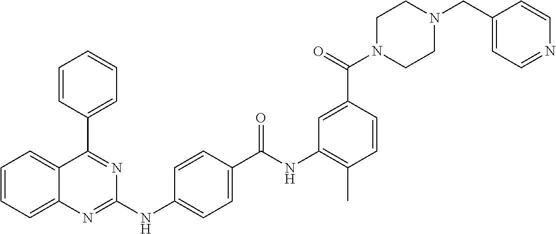 [SMILES: Cc1ccc(C(=O)N2CCN(Cc3ccncc3)CC2)cc1NC(=O)c1ccc(Nc2nc(-c3ccccc3)c3ccccc3n2)cc1]

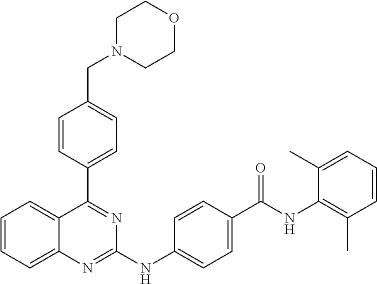 [SMILES: Cc1cccc(C)c1NC(=O)c1ccc(Nc2nc(-c3ccc(CN4CCOCC4)cc3)c3ccccc3n2)cc1]

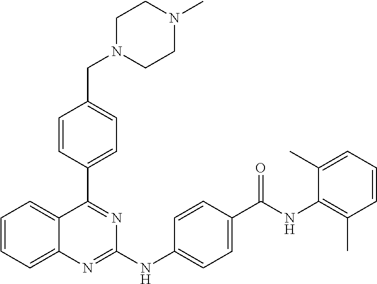 [SMILES: Cc1cccc(C)c1NC(=O)c1ccc(Nc2nc(-c3ccc(CN4CCN(C)CC4)cc3)c3ccccc3n2)cc1]

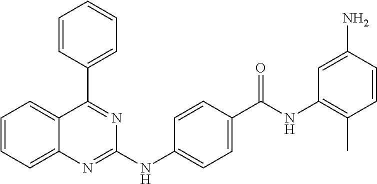 [SMILES: Cc1ccc(N)cc1NC(=O)c1ccc(Nc2nc(-c3ccccc3)c3ccccc3n2)cc1]

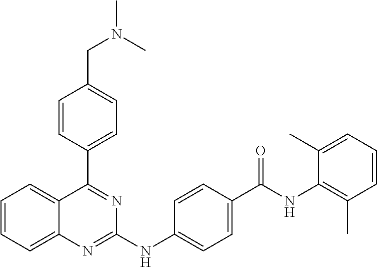 [SMILES: Cc1cccc(C)c1NC(=O)c1ccc(Nc2nc(-c3ccc(CN(C)C)cc3)c3ccccc3n2)cc1]

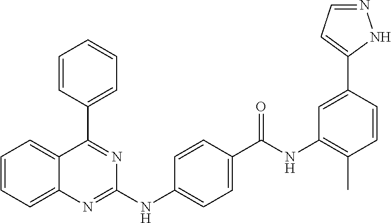 [SMILES: Cc1ccc(-c2ccn[nH]2)cc1NC(=O)c1ccc(Nc2nc(-c3ccccc3)c3ccccc3n2)cc1]

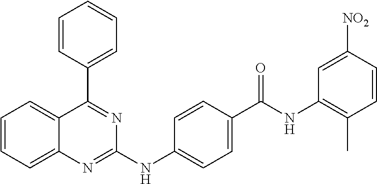 [SMILES: Cc1ccc([N+](=O)[O-])cc1NC(=O)c1ccc(Nc2nc(-c3ccccc3)c3ccccc3n2)cc1]

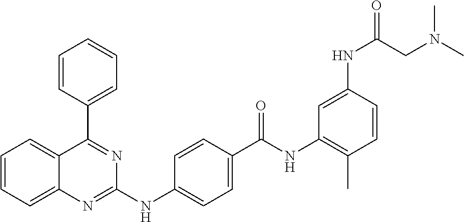 [SMILES: Cc1ccc(NC(=O)CN(C)C)cc1NC(=O)c1ccc(Nc2nc(-c3ccccc3)c3ccccc3n2)cc1]

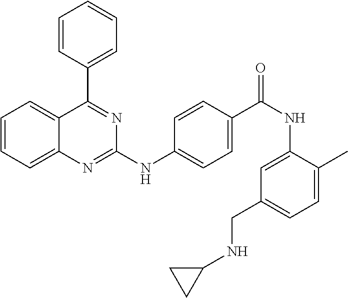 [SMILES: Cc1ccc(CNC2CC2)cc1NC(=O)c1ccc(Nc2nc(-c3ccccc3)c3ccccc3n2)cc1]

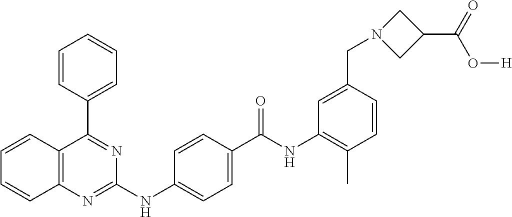 [SMILES: Cc1ccc(CN2CC(C(=O)O)C2)cc1NC(=O)c1ccc(Nc2nc(-c3ccccc3)c3ccccc3n2)cc1]

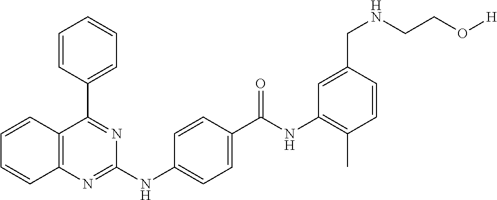 [SMILES: Cc1ccc(CNCCO)cc1NC(=O)c1ccc(Nc2nc(-c3ccccc3)c3ccccc3n2)cc1]

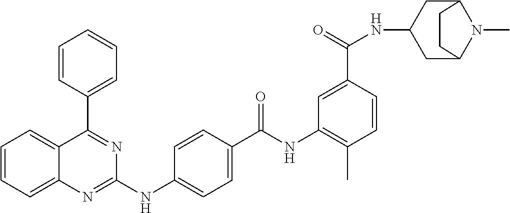 [SMILES: Cc1ccc(C(=O)NC2CC3CCC(C2)N3C)cc1NC(=O)c1ccc(Nc2nc(-c3ccccc3)c3ccccc3n2)cc1]

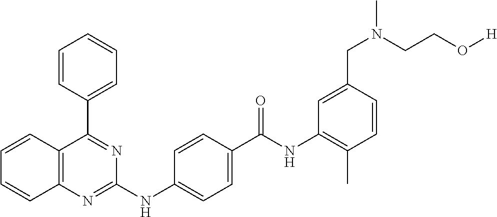 [SMILES: Cc1ccc(CN(C)CCO)cc1NC(=O)c1ccc(Nc2nc(-c3ccccc3)c3ccccc3n2)cc1]

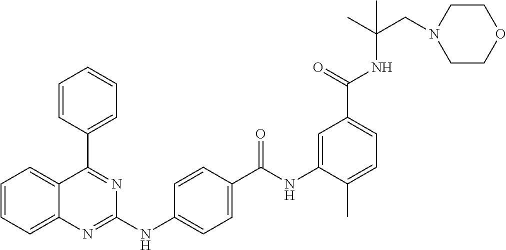 [SMILES: Cc1ccc(C(=O)NC(C)(C)CN2CCOCC2)cc1NC(=O)c1ccc(Nc2nc(-c3ccccc3)c3ccccc3n2)cc1]

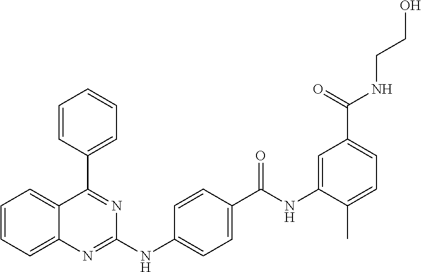 [SMILES: Cc1ccc(C(=O)NCCO)cc1NC(=O)c1ccc(Nc2nc(-c3ccccc3)c3ccccc3n2)cc1]

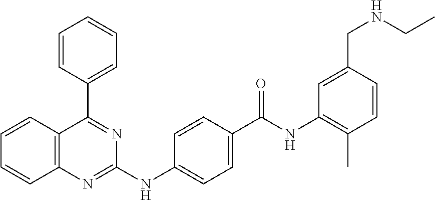 [SMILES: CCNCc1ccc(C)c(NC(=O)c2ccc(Nc3nc(-c4ccccc4)c4ccccc4n3)cc2)c1]

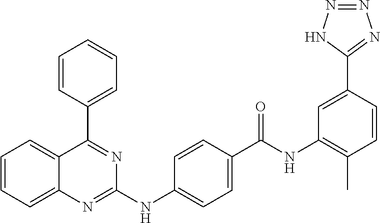 [SMILES: Cc1ccc(-c2nnn[nH]2)cc1NC(=O)c1ccc(Nc2nc(-c3ccccc3)c3ccccc3n2)cc1]

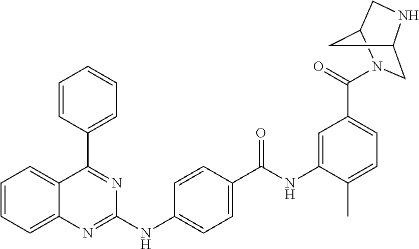 [SMILES: Cc1ccc(C(=O)N2CC3CC2CN3)cc1NC(=O)c1ccc(Nc2nc(-c3ccccc3)c3ccccc3n2)cc1]

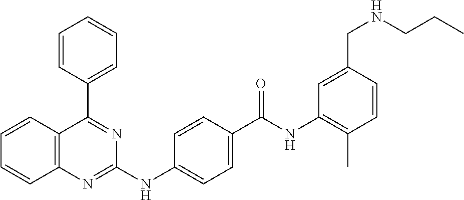 [SMILES: CCCNCc1ccc(C)c(NC(=O)c2ccc(Nc3nc(-c4ccccc4)c4ccccc4n3)cc2)c1]

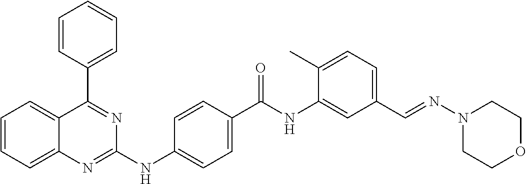 [SMILES: Cc1ccc(/C=N/N2CCOCC2)cc1NC(=O)c1ccc(Nc2nc(-c3ccccc3)c3ccccc3n2)cc1]

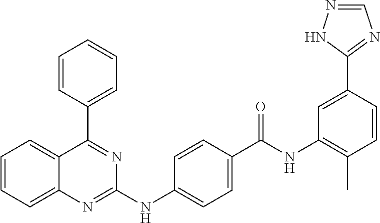 [SMILES: Cc1ccc(-c2ncn[nH]2)cc1NC(=O)c1ccc(Nc2nc(-c3ccccc3)c3ccccc3n2)cc1]

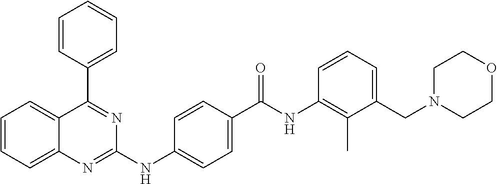 [SMILES: Cc1c(CN2CCOCC2)cccc1NC(=O)c1ccc(Nc2nc(-c3ccccc3)c3ccccc3n2)cc1]

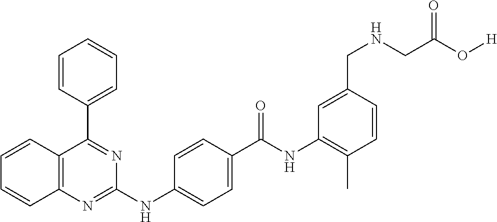 [SMILES: Cc1ccc(CNCC(=O)O)cc1NC(=O)c1ccc(Nc2nc(-c3ccccc3)c3ccccc3n2)cc1]

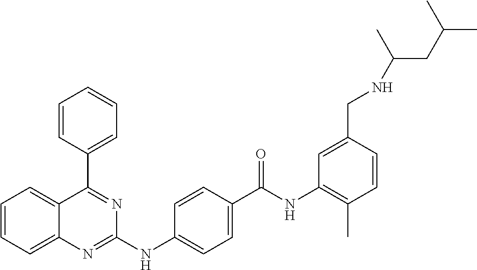 [SMILES: Cc1ccc(CNC(C)CC(C)C)cc1NC(=O)c1ccc(Nc2nc(-c3ccccc3)c3ccccc3n2)cc1]

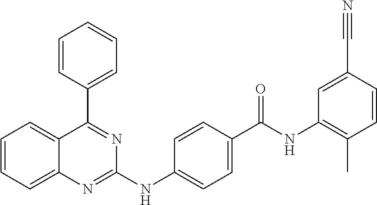 [SMILES: Cc1ccc(C#N)cc1NC(=O)c1ccc(Nc2nc(-c3ccccc3)c3ccccc3n2)cc1]

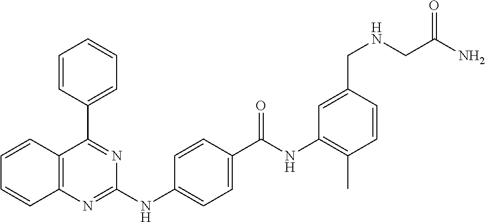 [SMILES: Cc1ccc(CNCC(N)=O)cc1NC(=O)c1ccc(Nc2nc(-c3ccccc3)c3ccccc3n2)cc1]

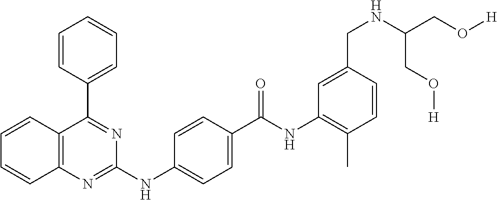 [SMILES: Cc1ccc(CNC(CO)CO)cc1NC(=O)c1ccc(Nc2nc(-c3ccccc3)c3ccccc3n2)cc1]

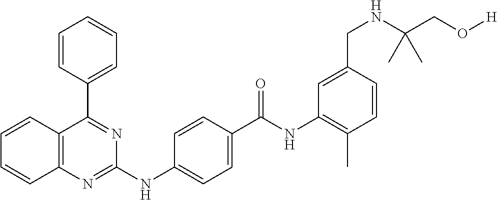 [SMILES: Cc1ccc(CNC(C)(C)CO)cc1NC(=O)c1ccc(Nc2nc(-c3ccccc3)c3ccccc3n2)cc1]

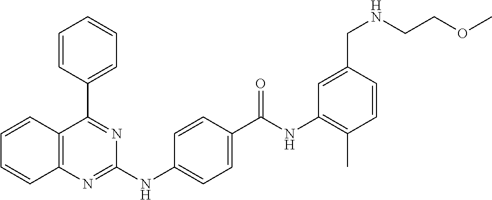 [SMILES: COCCNCc1ccc(C)c(NC(=O)c2ccc(Nc3nc(-c4ccccc4)c4ccccc4n3)cc2)c1]

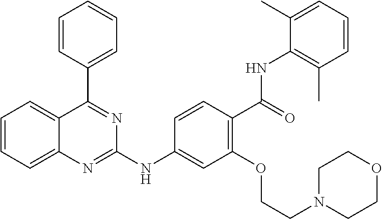 [SMILES: Cc1cccc(C)c1NC(=O)c1ccc(Nc2nc(-c3ccccc3)c3ccccc3n2)cc1OCCN1CCOCC1]

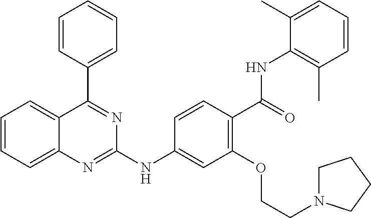 [SMILES: Cc1cccc(C)c1NC(=O)c1ccc(Nc2nc(-c3ccccc3)c3ccccc3n2)cc1OCCN1CCCC1]

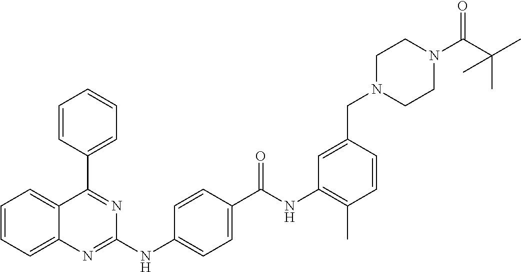 [SMILES: Cc1ccc(CN2CCN(C(=O)C(C)(C)C)CC2)cc1NC(=O)c1ccc(Nc2nc(-c3ccccc3)c3ccccc3n2)cc1]

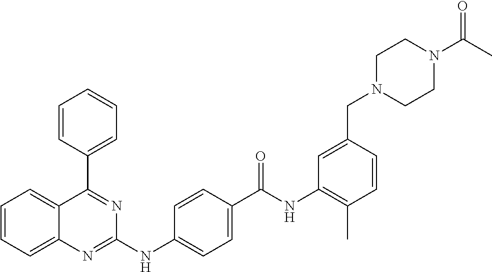 [SMILES: CC(=O)N1CCN(Cc2ccc(C)c(NC(=O)c3ccc(Nc4nc(-c5ccccc5)c5ccccc5n4)cc3)c2)CC1]

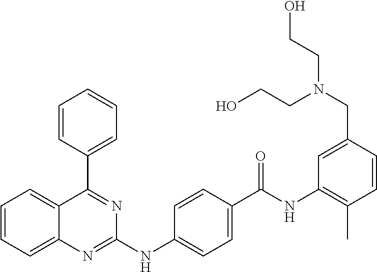 [SMILES: Cc1ccc(CN(CCO)CCO)cc1NC(=O)c1ccc(Nc2nc(-c3ccccc3)c3ccccc3n2)cc1]

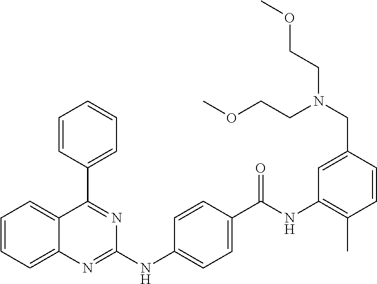 [SMILES: COCCN(CCOC)Cc1ccc(C)c(NC(=O)c2ccc(Nc3nc(-c4ccccc4)c4ccccc4n3)cc2)c1]

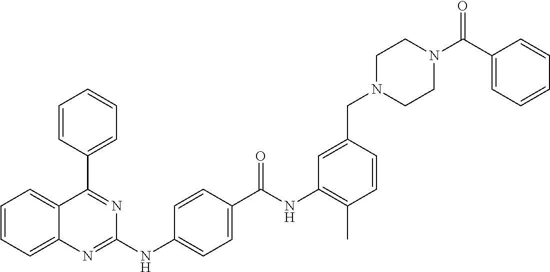 [SMILES: Cc1ccc(CN2CCN(C(=O)c3ccccc3)CC2)cc1NC(=O)c1ccc(Nc2nc(-c3ccccc3)c3ccccc3n2)cc1]

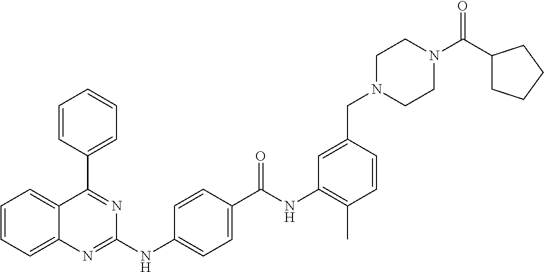 [SMILES: Cc1ccc(CN2CCN(C(=O)C3CCCC3)CC2)cc1NC(=O)c1ccc(Nc2nc(-c3ccccc3)c3ccccc3n2)cc1]

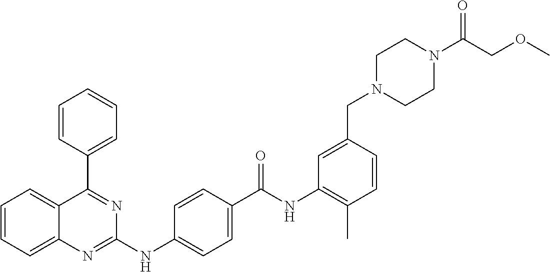 [SMILES: COCC(=O)N1CCN(Cc2ccc(C)c(NC(=O)c3ccc(Nc4nc(-c5ccccc5)c5ccccc5n4)cc3)c2)CC1]

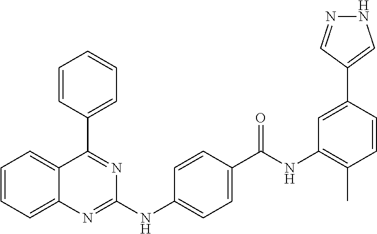 [SMILES: Cc1ccc(-c2cn[nH]c2)cc1NC(=O)c1ccc(Nc2nc(-c3ccccc3)c3ccccc3n2)cc1]

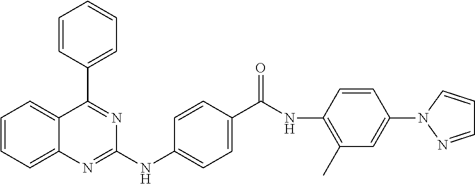 [SMILES: Cc1cc(-n2cccn2)ccc1NC(=O)c1ccc(Nc2nc(-c3ccccc3)c3ccccc3n2)cc1]

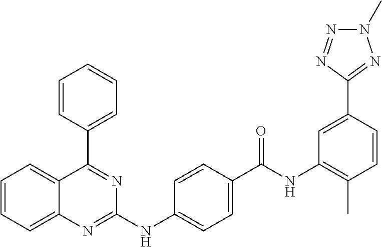 [SMILES: Cc1ccc(-c2nnn(C)n2)cc1NC(=O)c1ccc(Nc2nc(-c3ccccc3)c3ccccc3n2)cc1]

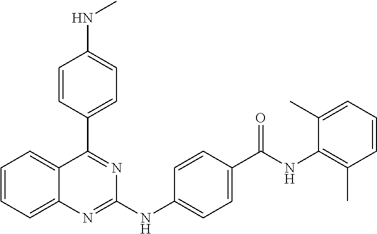 [SMILES: CNc1ccc(-c2nc(Nc3ccc(C(=O)Nc4c(C)cccc4C)cc3)nc3ccccc23)cc1]

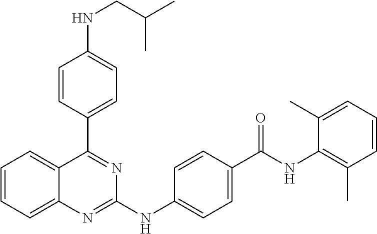 [SMILES: Cc1cccc(C)c1NC(=O)c1ccc(Nc2nc(-c3ccc(NCC(C)C)cc3)c3ccccc3n2)cc1]

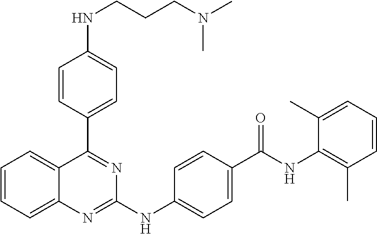 [SMILES: Cc1cccc(C)c1NC(=O)c1ccc(Nc2nc(-c3ccc(NCCCN(C)C)cc3)c3ccccc3n2)cc1]

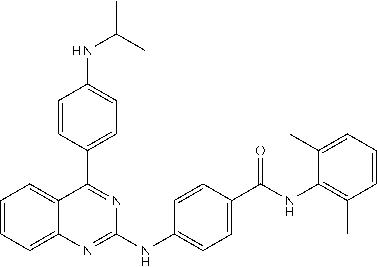 [SMILES: Cc1cccc(C)c1NC(=O)c1ccc(Nc2nc(-c3ccc(NC(C)C)cc3)c3ccccc3n2)cc1]

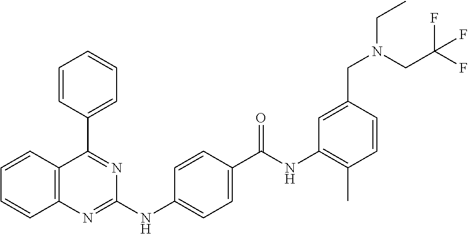 [SMILES: CCN(Cc1ccc(C)c(NC(=O)c2ccc(Nc3nc(-c4ccccc4)c4ccccc4n3)cc2)c1)CC(F)(F)F]

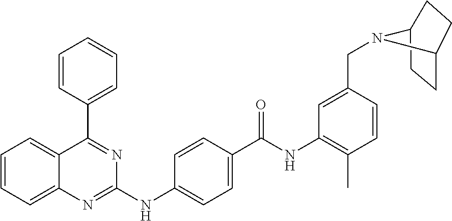 [SMILES: Cc1ccc(CN2C3CCC2CC3)cc1NC(=O)c1ccc(Nc2nc(-c3ccccc3)c3ccccc3n2)cc1]

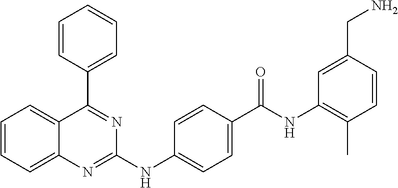 [SMILES: Cc1ccc(CN)cc1NC(=O)c1ccc(Nc2nc(-c3ccccc3)c3ccccc3n2)cc1]

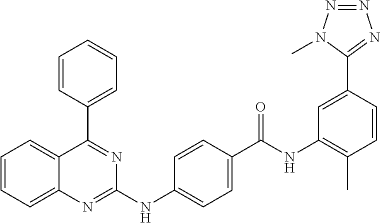 [SMILES: Cc1ccc(-c2nnnn2C)cc1NC(=O)c1ccc(Nc2nc(-c3ccccc3)c3ccccc3n2)cc1]

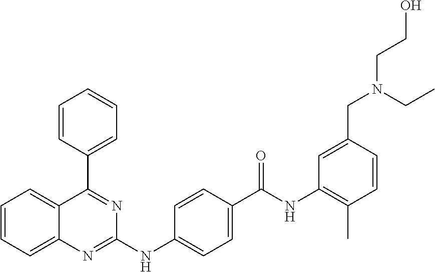 [SMILES: CCN(CCO)Cc1ccc(C)c(NC(=O)c2ccc(Nc3nc(-c4ccccc4)c4ccccc4n3)cc2)c1]

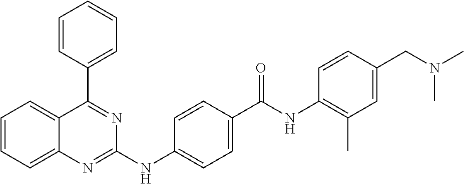 [SMILES: Cc1cc(CN(C)C)ccc1NC(=O)c1ccc(Nc2nc(-c3ccccc3)c3ccccc3n2)cc1]

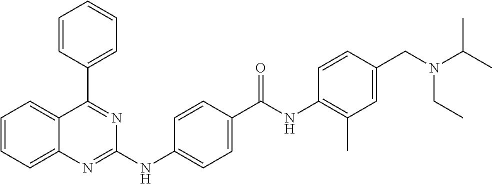 [SMILES: CCN(Cc1ccc(NC(=O)c2ccc(Nc3nc(-c4ccccc4)c4ccccc4n3)cc2)c(C)c1)C(C)C]

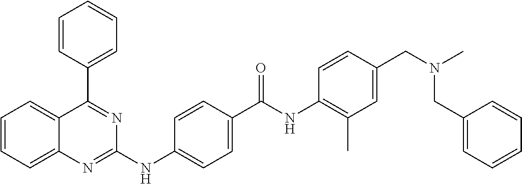 [SMILES: Cc1cc(CN(C)Cc2ccccc2)ccc1NC(=O)c1ccc(Nc2nc(-c3ccccc3)c3ccccc3n2)cc1]

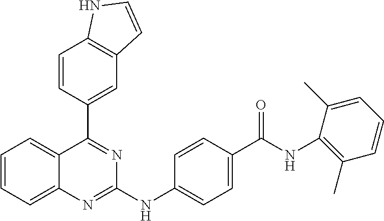 [SMILES: Cc1cccc(C)c1NC(=O)c1ccc(Nc2nc(-c3ccc4[nH]ccc4c3)c3ccccc3n2)cc1]

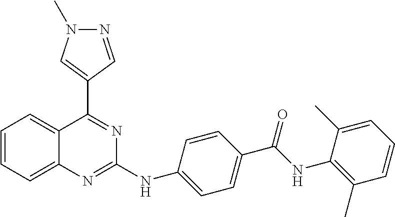 [SMILES: Cc1cccc(C)c1NC(=O)c1ccc(Nc2nc(-c3cnn(C)c3)c3ccccc3n2)cc1]

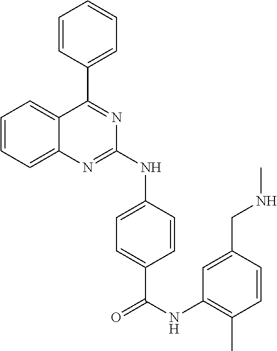 [SMILES: CNCc1ccc(C)c(NC(=O)c2ccc(Nc3nc(-c4ccccc4)c4ccccc4n3)cc2)c1]

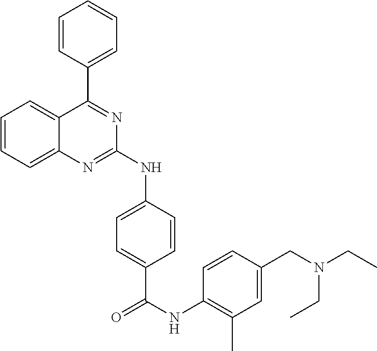 [SMILES: CCN(CC)Cc1ccc(NC(=O)c2ccc(Nc3nc(-c4ccccc4)c4ccccc4n3)cc2)c(C)c1]

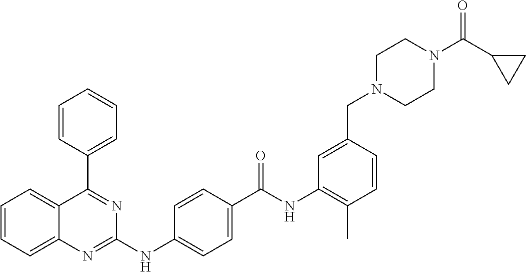 [SMILES: Cc1ccc(CN2CCN(C(=O)C3CC3)CC2)cc1NC(=O)c1ccc(Nc2nc(-c3ccccc3)c3ccccc3n2)cc1]

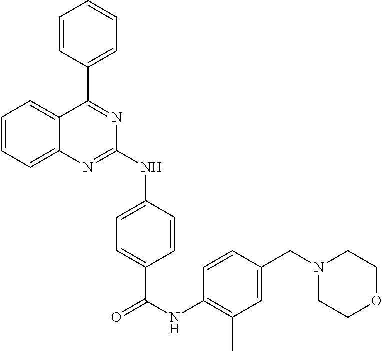 [SMILES: Cc1cc(CN2CCOCC2)ccc1NC(=O)c1ccc(Nc2nc(-c3ccccc3)c3ccccc3n2)cc1]

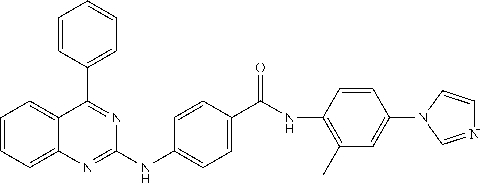 [SMILES: Cc1cc(-n2ccnc2)ccc1NC(=O)c1ccc(Nc2nc(-c3ccccc3)c3ccccc3n2)cc1]

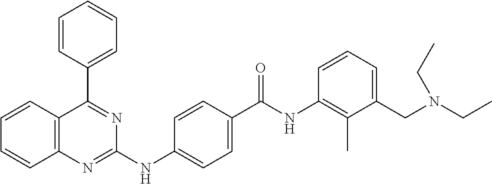 [SMILES: CCN(CC)Cc1cccc(NC(=O)c2ccc(Nc3nc(-c4ccccc4)c4ccccc4n3)cc2)c1C]

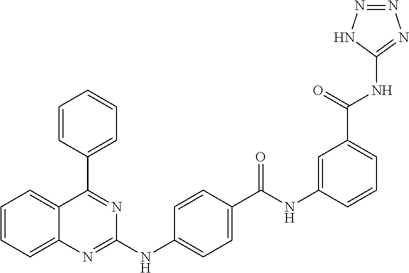 [SMILES: O=C(Nc1cccc(C(=O)Nc2nnn[nH]2)c1)c1ccc(Nc2nc(-c3ccccc3)c3ccccc3n2)cc1]

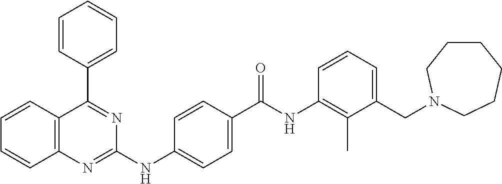 [SMILES: Cc1c(CN2CCCCCC2)cccc1NC(=O)c1ccc(Nc2nc(-c3ccccc3)c3ccccc3n2)cc1]

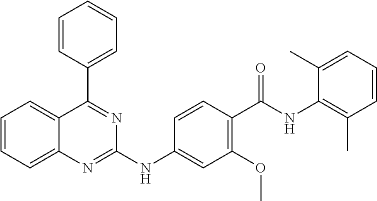 [SMILES: COc1cc(Nc2nc(-c3ccccc3)c3ccccc3n2)ccc1C(=O)Nc1c(C)cccc1C]

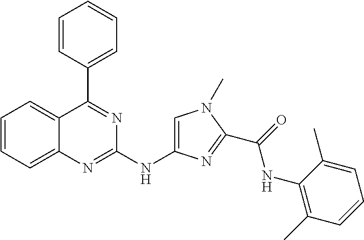 [SMILES: Cc1cccc(C)c1NC(=O)c1nc(Nc2nc(-c3ccccc3)c3ccccc3n2)cn1C]

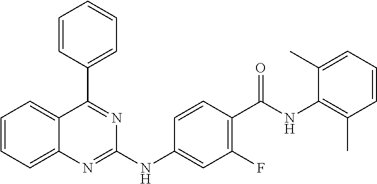 [SMILES: Cc1cccc(C)c1NC(=O)c1ccc(Nc2nc(-c3ccccc3)c3ccccc3n2)cc1F]

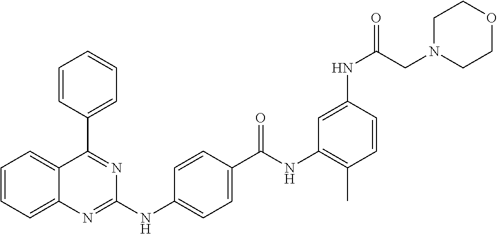 [SMILES: Cc1ccc(NC(=O)CN2CCOCC2)cc1NC(=O)c1ccc(Nc2nc(-c3ccccc3)c3ccccc3n2)cc1]

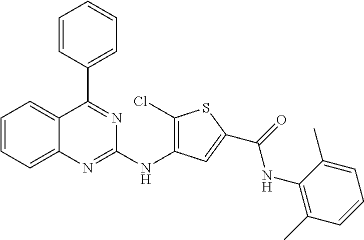 [SMILES: Cc1cccc(C)c1NC(=O)c1cc(Nc2nc(-c3ccccc3)c3ccccc3n2)c(Cl)s1]

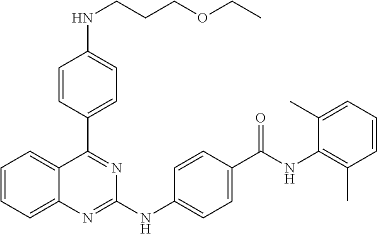 [SMILES: CCOCCCNc1ccc(-c2nc(Nc3ccc(C(=O)Nc4c(C)cccc4C)cc3)nc3ccccc23)cc1]

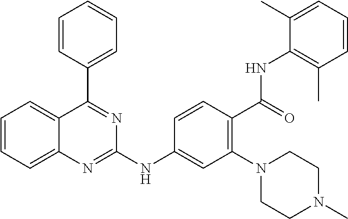 [SMILES: Cc1cccc(C)c1NC(=O)c1ccc(Nc2nc(-c3ccccc3)c3ccccc3n2)cc1N1CCN(C)CC1]

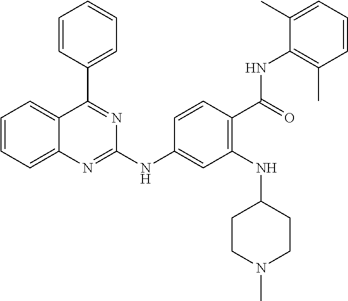 [SMILES: Cc1cccc(C)c1NC(=O)c1ccc(Nc2nc(-c3ccccc3)c3ccccc3n2)cc1NC1CCN(C)CC1]

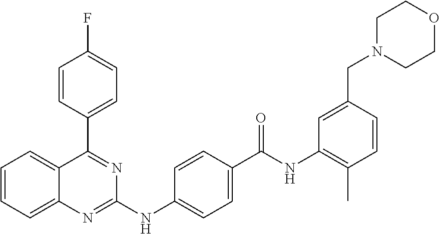 [SMILES: Cc1ccc(CN2CCOCC2)cc1NC(=O)c1ccc(Nc2nc(-c3ccc(F)cc3)c3ccccc3n2)cc1]

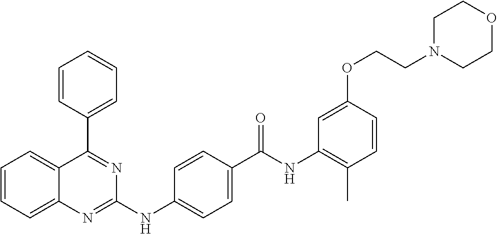 [SMILES: Cc1ccc(OCCN2CCOCC2)cc1NC(=O)c1ccc(Nc2nc(-c3ccccc3)c3ccccc3n2)cc1]

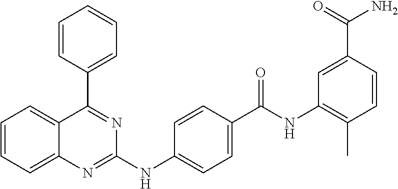 [SMILES: Cc1ccc(C(N)=O)cc1NC(=O)c1ccc(Nc2nc(-c3ccccc3)c3ccccc3n2)cc1]

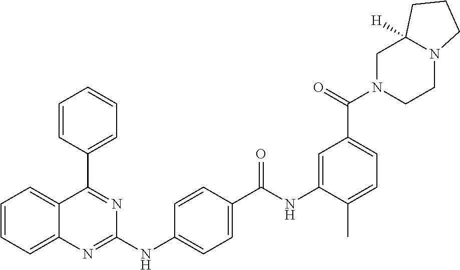 [SMILES: Cc1ccc(C(=O)N2CCN3CCC[C@@H]3C2)cc1NC(=O)c1ccc(Nc2nc(-c3ccccc3)c3ccccc3n2)cc1]

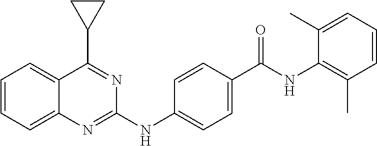 [SMILES: Cc1cccc(C)c1NC(=O)c1ccc(Nc2nc(C3CC3)c3ccccc3n2)cc1]

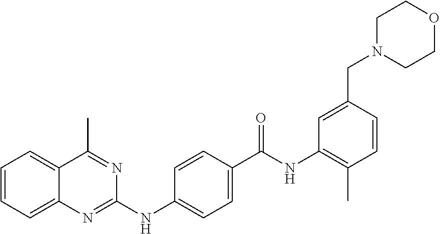 [SMILES: Cc1ccc(CN2CCOCC2)cc1NC(=O)c1ccc(Nc2nc(C)c3ccccc3n2)cc1]

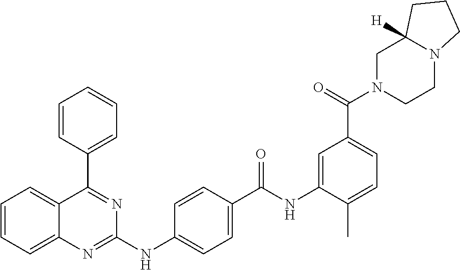 [SMILES: Cc1ccc(C(=O)N2CCN3CCC[C@H]3C2)cc1NC(=O)c1ccc(Nc2nc(-c3ccccc3)c3ccccc3n2)cc1]

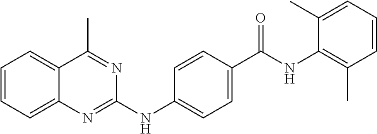 [SMILES: Cc1cccc(C)c1NC(=O)c1ccc(Nc2nc(C)c3ccccc3n2)cc1]

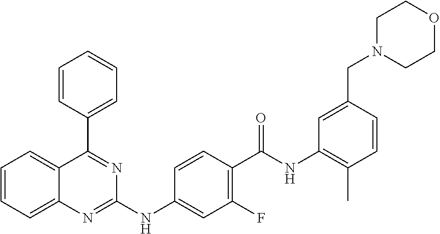 [SMILES: Cc1ccc(CN2CCOCC2)cc1NC(=O)c1ccc(Nc2nc(-c3ccccc3)c3ccccc3n2)cc1F]

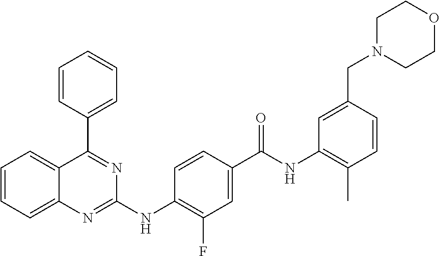 [SMILES: Cc1ccc(CN2CCOCC2)cc1NC(=O)c1ccc(Nc2nc(-c3ccccc3)c3ccccc3n2)c(F)c1]

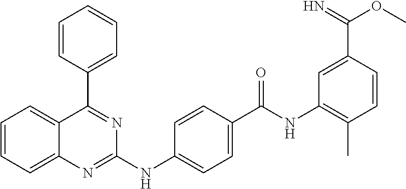 [SMILES: COC(=N)c1ccc(C)c(NC(=O)c2ccc(Nc3nc(-c4ccccc4)c4ccccc4n3)cc2)c1]

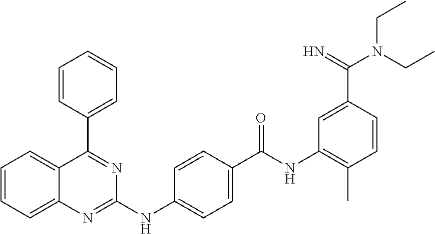 [SMILES: CCN(CC)C(=N)c1ccc(C)c(NC(=O)c2ccc(Nc3nc(-c4ccccc4)c4ccccc4n3)cc2)c1]

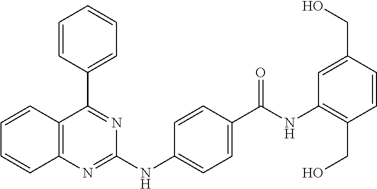 [SMILES: O=C(Nc1cc(CO)ccc1CO)c1ccc(Nc2nc(-c3ccccc3)c3ccccc3n2)cc1]

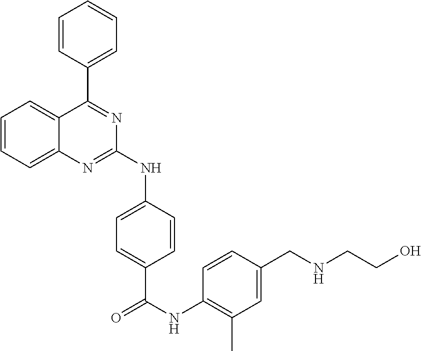 [SMILES: Cc1cc(CNCCO)ccc1NC(=O)c1ccc(Nc2nc(-c3ccccc3)c3ccccc3n2)cc1]

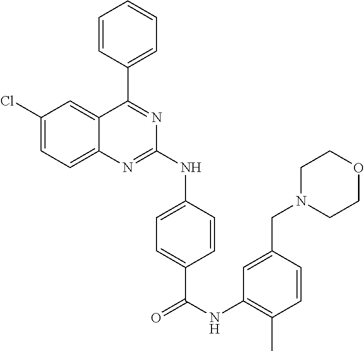 [SMILES: Cc1ccc(CN2CCOCC2)cc1NC(=O)c1ccc(Nc2nc(-c3ccccc3)c3cc(Cl)ccc3n2)cc1]

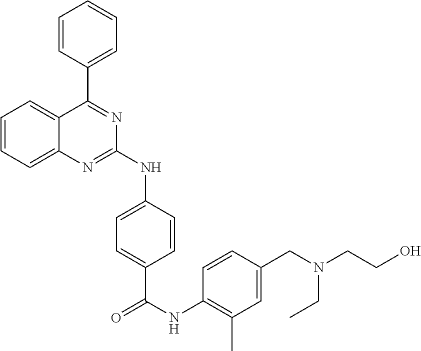 [SMILES: CCN(CCO)Cc1ccc(NC(=O)c2ccc(Nc3nc(-c4ccccc4)c4ccccc4n3)cc2)c(C)c1]

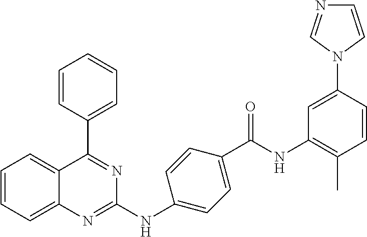 [SMILES: Cc1ccc(-n2ccnc2)cc1NC(=O)c1ccc(Nc2nc(-c3ccccc3)c3ccccc3n2)cc1]

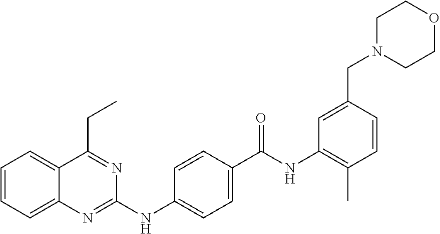 [SMILES: CCc1nc(Nc2ccc(C(=O)Nc3cc(CN4CCOCC4)ccc3C)cc2)nc2ccccc12]

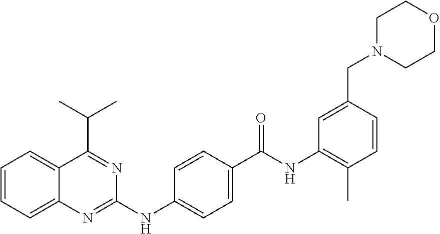 [SMILES: Cc1ccc(CN2CCOCC2)cc1NC(=O)c1ccc(Nc2nc(C(C)C)c3ccccc3n2)cc1]

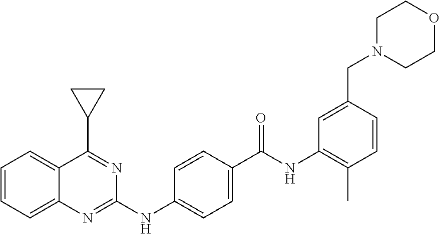 [SMILES: Cc1ccc(CN2CCOCC2)cc1NC(=O)c1ccc(Nc2nc(C3CC3)c3ccccc3n2)cc1]

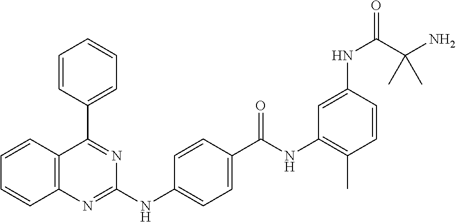 [SMILES: Cc1ccc(NC(=O)C(C)(C)N)cc1NC(=O)c1ccc(Nc2nc(-c3ccccc3)c3ccccc3n2)cc1]

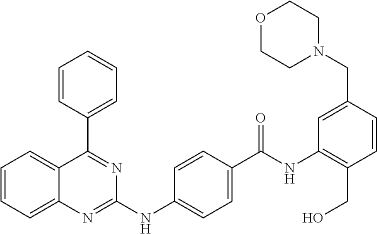 [SMILES: O=C(Nc1cc(CN2CCOCC2)ccc1CO)c1ccc(Nc2nc(-c3ccccc3)c3ccccc3n2)cc1]

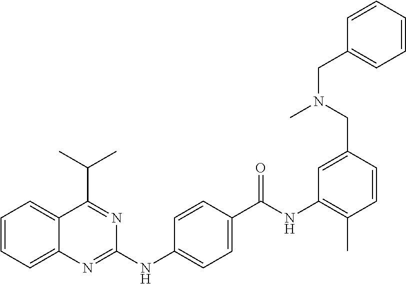 [SMILES: Cc1ccc(CN(C)Cc2ccccc2)cc1NC(=O)c1ccc(Nc2nc(C(C)C)c3ccccc3n2)cc1]

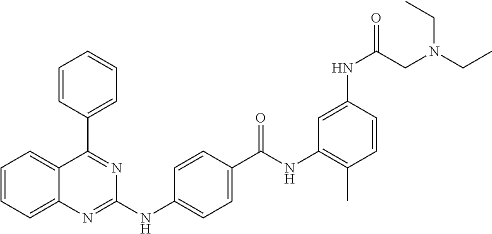 [SMILES: CCN(CC)CC(=O)Nc1ccc(C)c(NC(=O)c2ccc(Nc3nc(-c4ccccc4)c4ccccc4n3)cc2)c1]

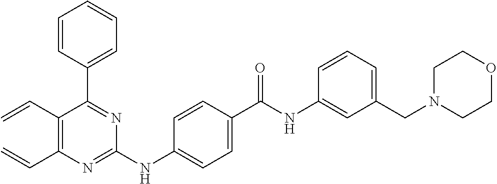 [SMILES: C=Cc1nc(Nc2ccc(C(=O)Nc3cccc(CN4CCOCC4)c3)cc2)nc(-c2ccccc2)c1C=C]

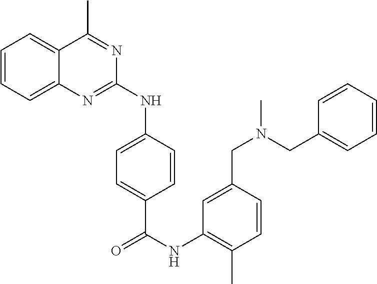 [SMILES: Cc1ccc(CN(C)Cc2ccccc2)cc1NC(=O)c1ccc(Nc2nc(C)c3ccccc3n2)cc1]

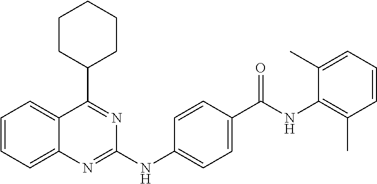 [SMILES: Cc1cccc(C)c1NC(=O)c1ccc(Nc2nc(C3CCCCC3)c3ccccc3n2)cc1]

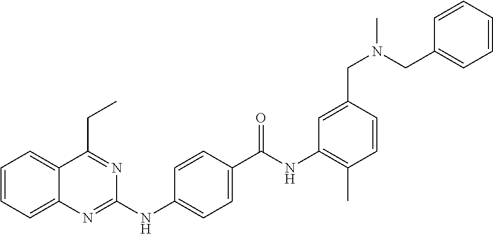 [SMILES: CCc1nc(Nc2ccc(C(=O)Nc3cc(CN(C)Cc4ccccc4)ccc3C)cc2)nc2ccccc12]

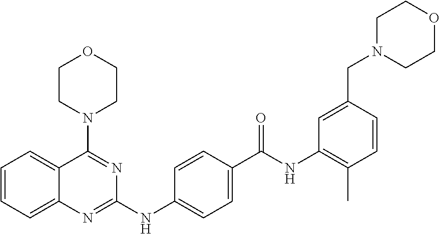 [SMILES: Cc1ccc(CN2CCOCC2)cc1NC(=O)c1ccc(Nc2nc(N3CCOCC3)c3ccccc3n2)cc1]

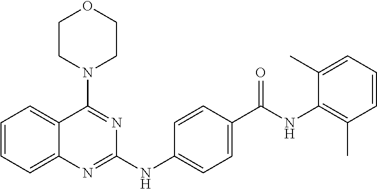 [SMILES: Cc1cccc(C)c1NC(=O)c1ccc(Nc2nc(N3CCOCC3)c3ccccc3n2)cc1]

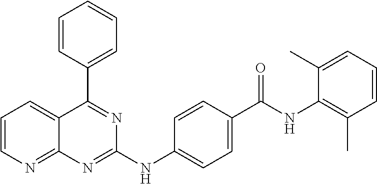 [SMILES: Cc1cccc(C)c1NC(=O)c1ccc(Nc2nc(-c3ccccc3)c3cccnc3n2)cc1]

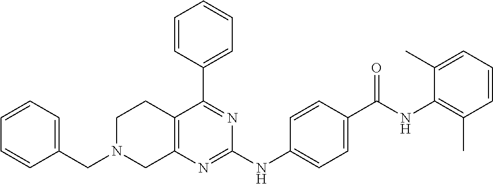 [SMILES: Cc1cccc(C)c1NC(=O)c1ccc(Nc2nc3c(c(-c4ccccc4)n2)CCN(Cc2ccccc2)C3)cc1]

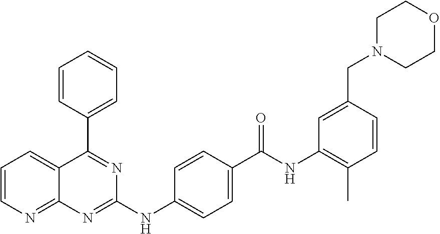 [SMILES: Cc1ccc(CN2CCOCC2)cc1NC(=O)c1ccc(Nc2nc(-c3ccccc3)c3cccnc3n2)cc1]

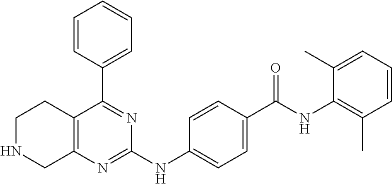 [SMILES: Cc1cccc(C)c1NC(=O)c1ccc(Nc2nc3c(c(-c4ccccc4)n2)CCNC3)cc1]

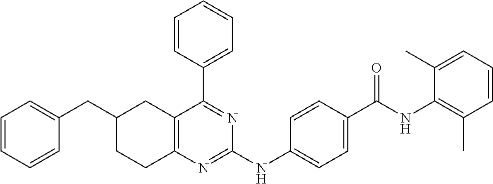 [SMILES: Cc1cccc(C)c1NC(=O)c1ccc(Nc2nc3c(c(-c4ccccc4)n2)CC(Cc2ccccc2)CC3)cc1]

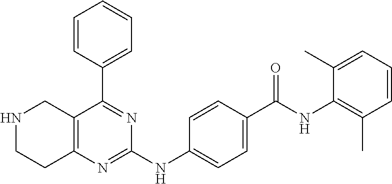 [SMILES: Cc1cccc(C)c1NC(=O)c1ccc(Nc2nc3c(c(-c4ccccc4)n2)CNCC3)cc1]

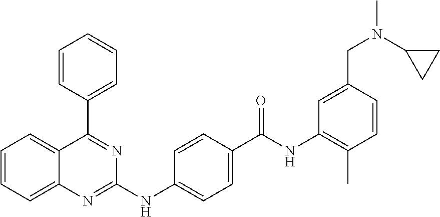 [SMILES: Cc1ccc(CN(C)C2CC2)cc1NC(=O)c1ccc(Nc2nc(-c3ccccc3)c3ccccc3n2)cc1]

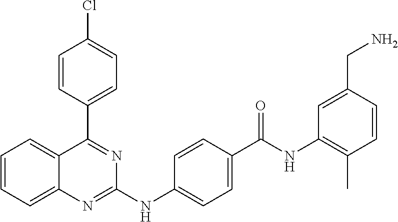 [SMILES: Cc1ccc(CN)cc1NC(=O)c1ccc(Nc2nc(-c3ccc(Cl)cc3)c3ccccc3n2)cc1]

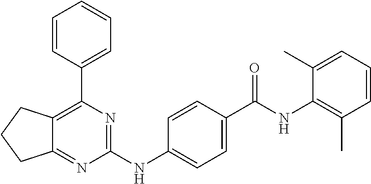 [SMILES: Cc1cccc(C)c1NC(=O)c1ccc(Nc2nc3c(c(-c4ccccc4)n2)CCC3)cc1]

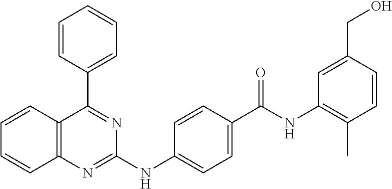 [SMILES: Cc1ccc(CO)cc1NC(=O)c1ccc(Nc2nc(-c3ccccc3)c3ccccc3n2)cc1]

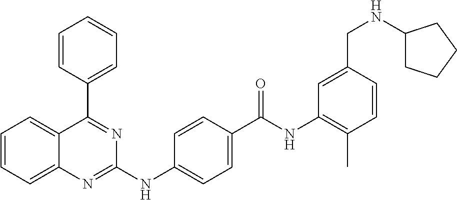 [SMILES: Cc1ccc(CNC2CCCC2)cc1NC(=O)c1ccc(Nc2nc(-c3ccccc3)c3ccccc3n2)cc1]

### Methods of Treatment

Some embodiments of the present disclosure relate to methods of killing or retarding the growth of a neoplastic cell, methods of increasing the sensitivity of a cell to a chemotherapeutic agent, methods of ameliorating cancer in a subject, methods of increasing the sensitivity of a cancer in a subject to chemotherapeutic compounds, and methods of reducing the dosage of a chemotherapeutic agent needed by a subject. Some embodiments provided herein include the use of an effective amount of a SMO inhibitor for reducing the dosage of a chemotherapeutic agent needed to ameliorate cancer in a subject. Some embodiments provided herein include the use of an effective amount a SMO inhibitor for increasing the sensitivity of a cancer to a chemotherapeutic compound, wherein the effective amount of the chemotherapeutic compound is significantly less than the effective amount of the chemotherapeutic compound in the absence of the SMO inhibitor. Some embodiments provided herein include the use of an effective amount of a SMO inhibitor in combination with an effective amount of a chemotherapeutic agent for ameliorating cancer in a subject in need thereof. In particular embodiments, the chemotherapeutic compound comprises a platinum-based therapeutic compound.

Some embodiments, include contacting a cell or administering to a subject an effective amount of a SMO inhibitor in combination with an effective amount of a chemotherapeutic agent. Examples of SMO inhibitors useful with the methods described herein include BMS-833923, compounds of Table 3, cyclopamine, agents that decrease the expression of the SMO protein, and agents that increase expression of the SMO inhibitor PTCH protein. Examples of SMO inhibitors are disclosed in U.S. Patent Application No. 2009/0105211, the contents of which are incorporated herein by reference in its entirety. Examples of chemotherapeutic agents useful with the methods described herein include platinum-based compounds such as cisplatin, carboplatin, nedaplatin, oxaliplatin, satraplatin, and triplatin tetranitrate, nitrogen mustards such as cyclophosphamide, mechlorethamine, uramustine, melphalan, chlorambucil, and ifosfamide, nitrosoureas such as carmustine, lomustine, and streptozocin, alkyl sulfonates such as busulfan, thiotepa, procarbazine, and altretamine. More examples of chemotherapeutic agents include taxol, gemcitabine, toptecan hydrochloride, doxorubicin and pegylated doxorubicin.

In some embodiments, the effective amount of the chemotherapeutic compound is less than the effective amount of the chemotherapeutic compound in the absence of the SMO inhibitor by at least about 10%, 20%, 30%, 40%, 50%, 60%, 70%, 80%, 90%, 100%, or more. In some embodiments, the effective amount of the SMO inhibitor is an amount which significantly reduces the IC50 of the chemotherapeutic compound. In some such embodiments, the IC50 of the chemotherapeutic agent is reduced by at least about 10%, 20%, 30%, 40%, 50%, 60%, 70%, 80%, 90%, 100%, or more. In some embodiments, the effective amount of the chemotherapeutic compound and the effective amount of the SMO inhibitor have a Combination Index less than 1 as determined by Calcusyn software (Version 2, BIOSOFT, Cambridge U.K.).

Some embodiments include methods of increasing the sensitivity of a neoplastic cell to a chemotherapeutic compound. Some such methods include contacting the cell with an effective amount a SMO inhibitor and an effective amount of the chemotherapeutic compound, wherein the effective amount of the chemotherapeutic compound is significantly less than the effective amount of the chemotherapeutic compound in the absence of the SMO inhibitor. In some embodiments the SMO inhibitor comprises BMS-833923. In some embodiments the SMO inhibitor comprises a compound of Table 3. In some embodiments, the cell is contacted in vitro. In some embodiments, the cell is contacted in vivo. In some embodiments, the cell comprises an ovarian cancer cell. In some embodiments, the cell comprises a platinum resistant ovarian cancer cell. In some embodiments, the cell comprises an ovarian cancer stem cell. In some embodiments, the cell is mammalian, for example, human.

Contacting a cell with a SMO inhibitor, such as BMS-833923 or a compound of Table 3, in combination with a chemotherapeutic agent can include contacting the cell with the SMO inhibitor and the chemotherapeutic agent at the same time, at different times, and at overlapping time periods. The cell may be contacted with the SMO inhibitor before, after, or during the period of time the cell is contacted with the chemotherapeutic agent. In some embodiments, contacting the cell with the chemotherapeutic agent commences before contacting the cell with the SMO inhibitor, such as BMS-833923 or a compound of Table 3, and continues through the period when the cell is contacted with the SMO inhibitor. In some embodiments, contacting the cell with the chemotherapeutic agent is before contacting the cell with the SMO inhibitor, such as BMS-833923 or a compound of Table 3, and the chemotherapeutic agent and SMO inhibitor are not placed in contact with the cell at overlapping times. In some embodiments, contacting the cell with the SMO inhibitor, such as BMS-833923 or a compound of Table 3, commences before contacting the cell with the chemotherapeutic agent, and continues through the period when the cell is contacted with the chemotherapeutic agent. In some embodiments, contacting the cell with the SMO inhibitor, such as BMS-833923 or a compound of Table 3, is before contacting the cell with the chemotherapeutic agent, and the chemotherapeutic agent and SMO inhibitor are not placed in contact with the cell at overlapping times.

In some embodiments, the time period between contacting the cell with the chemotherapeutic agent and contacting the cell with the SMO inhibitor, such as BMS-833923 or a compound of Table 3, is less than or more than about 5 minutes, 10 minutes, 20 minutes, 30 minutes, 40 minutes, 50 minutes, or 60 minutes. In some embodiments, the period of time between contacting the cell with the chemotherapeutic agent and contacting the cell with the SMO inhibitor, such as BMS-833923 or a compound of Table 3, is less than or more than about 1 hour, 2 hours, 3 hours, 4 hours, 5 hours, 6 hours, 7 hours, 8 hours, 9 hours, 10 hours, 11 hours, or 12 hours. In some embodiments, the period of time between contacting the cell with the chemotherapeutic agent and contacting the cell with the SMO inhibitor, such as BMS-833923 or a compound of Table 3, is less than or more than about 12 hour, 24 hours, 36 hours, or 48 hours. In some embodiments, the cell is contacted in vitro. In some embodiments, the cell is contacted in vivo. In some embodiments, the cell comprises an ovarian cancer cell. In some embodiments, the cell comprises a platinum resistant ovarian cancer cell. In some embodiments, the cell comprises an ovarian cancer stem cell. In some embodiments, the cell is mammalian, e.g., human.

Some embodiments include methods of ameliorating cancer in a subject. Some such embodiments include administering an effective amount a SMO inhibitor and an effective amount of the chemotherapeutic compound to the subject, wherein the effective amount of the chemotherapeutic compound is significantly less than the effective amount of the chemotherapeutic compound in the absence of the SMO inhibitor. In some embodiments, the SMO inhibitor comprises BMS-833923 or a compound of Table 3. In some embodiments, the chemotherapeutic agent is an agent described herein. Some embodiments include methods for reducing the dosage of a chemotherapeutic agent needed to treat a cancer in a subject. Some such embodiments include administering an effective amount a SMO inhibitor and an effective amount of the chemotherapeutic compound to the subject, wherein the effective amount of the chemotherapeutic compound is significantly less than the effective amount of the chemotherapeutic compound in the absence of the SMO inhibitor. In some embodiments, the SMO inhibitor comprises BMS-833923 or a compound of Table 3. In some embodiments, the chemotherapeutic agent is an agent described herein. More embodiments include increasing the sensitivity of a cancer in a subject to a chemotherapeutic compound. Some such embodiments include administering an effective amount a SMO inhibitor and an effective amount of the chemotherapeutic compound to the subject, wherein the effective amount of the chemotherapeutic compound is significantly less than the effective amount of the chemotherapeutic compound in the absence of the SMO inhibitor.

Administrating a SMO inhibitor, such as BMS-833923 or a compound of Table 3, to a subject in combination with a chemotherapeutic agent can include administering the SMO inhibitor and the chemotherapeutic agent at the same time, at different times, and at overlapping time periods. The SMO inhibitor may be administered to the subject before, after, or during the period of time the cell is contacted with the chemotherapeutic agent. In some embodiments, administering the chemotherapeutic agent to the subject commences before administering the SMO inhibitor, such as BMS-833923 or a compound of Table 3, to the subject. In some embodiments, administering the chemotherapeutic agent to the subject is before administering the SMO inhibitor, such as BMS-833923 or a compound of Table 3 to the subject. In some embodiments, contacting the cell with the SMO inhibitor, such as BMS-833923 or a compound of Table 3, commences before contacting the cell with the chemotherapeutic agent. In some embodiments, contacting the cell with the SMO inhibitor, such as BMS-833923 or a compound of Table 3, is before contacting the cell with the chemotherapeutic agent.

In some embodiments, the time period between administering the chemotherapeutic agent to the subject and administering the SMO inhibitor, such as BMS-833923 or a compound of Table 3, to the subject is less than, or greater than, about 5 minutes, 10 minutes, 20 minutes, 30 minutes, 40 minutes, 50 minutes, or 60 minutes. In some embodiments, the time period between administering the chemotherapeutic agent to the subject and administering the SMO inhibitor, such as BMS-833923 or a compound of Table 3, to the subject, is less than, or more than, about 1 hour, 2 hours, 3 hours, 4 hours, 5 hours, 6 hours, 7 hours, 8 hours, 9 hours, 10 hours, 11 hours, or 12 hours. In some embodiments, the time period between administering the chemotherapeutic agent to the subject and administering the SMO inhibitor, such as BMS-833923 or a compound of Table 3, to the subject, is less than or more than about 12 hour, 24 hours, 36 hours, and 48 hours. In some embodiments, the cell is contacted in vitro. In some embodiments, the cell is contacted in vivo. In some embodiments, the cancer comprises ovarian cancer. In some embodiments, the cancer comprises a platinum resistant ovarian cancer cell. In some embodiments, the cancer comprises an ovarian cancer stem cell. In some embodiments, the subject is mammalian, e.g., human.

In some embodiments, the SMO inhibitor, such as BMS-833923 or a compound of Table 3, is administered at least about daily, at least about weekly, or at least about monthly. In some embodiments, the dosage of the SMO inhibitor, such as BMS-833923 or a compound of Table 3, administered to the subject comprises at least about or no more than about 1 mg, 2 mg, 3, mg, 4 mg, 5 mg, 6 mg, 7, mg, 8 mg, 9 mg, or 10 mg. In some embodiments, the dosage of the SMO inhibitor, such as BMS-833923 or a compound of Table 3, administered to the subject comprises at least about or no more than about 10 mg, 20 mg, 30, mg, 40 mg, 50 mg, 60 mg, 70, mg, 80 mg, 90 mg, or 100 mg. As would be understood by a person of ordinary skill in the art, the dose administered to a subject can be modulated according to the body mass of the subject.

Some embodiments include pharmaceutical compositions comprising a SMO inhibitor, such as BMS-833923 or a compound of Table 3, and/or a chemotherapeutic agent. Such pharmaceutical compositions can also include a suitable carrier. While any suitable carrier known to those of ordinary skill in the art may be employed in the pharmaceutical compositions described herein, the type of carrier will typically vary depending on the mode of administration. Compositions described herein may be formulated for any appropriate manner of administration, including for example, topical, oral, nasal, mucosal, intravenous, intracranial, intraperitoneal, subcutaneous and intramuscular administration. Carriers for use within such pharmaceutical compositions are biocompatible, and may also be biodegradable. In certain embodiments, the formulation preferably provides a relatively constant level of active component release.

The pharmaceutical compositions described herein can further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions described herein may be formulated as a lyophilizate. Exemplary components which may be included in pharmaceutical compositions are described in Remington's The Science and Practice of Pharmacy, 21st Ed., Lippincott Williams & Wilkins (2005), the disclosure of which is incorporated by reference in its entirety.

Pharmaceutical compositions described herein can be administered to a subject, such as a mammal, such as a human. Pharmaceutical compositions can be administered at a therapeutically effective amount. A “therapeutically effective amount” is a quantity of a chemical composition which achieves a desired effect in a subject being treated.

### Method for Identifying Therapeutic Compounds

Some embodiments include methods for identifying a therapeutic compound. Some such embodiments can include methods for determining whether a candidate agent for ameliorating cancer acts in synergy with a SMO inhibitor. Some such methods can include contacting a population of cells with a SMO inhibitor in combination with a test compound; and determining whether the level of cell survival in the population of cells contacted with the SMO inhibitor in combination with the test compound is significantly less than the combined level of cell survival in a population of cells contacted with the SMO inhibitor and a population of cells contacted with the test compound. In some embodiments, a significantly lower level of cell survival in the population of cells contacted with the SMO inhibitor in combination with the test compound compared to the combined level of cell survival in a population of cells contacted with the SMO inhibitor and a population of cells contacted with the test compound indicates that the test compound acts in synergy with the SMO inhibitor. The significantly lower level of cell survival in the population of cells contacted with the SMO inhibitor in combination with the test compound can include a decrease in the combined level of cell survival of a population of cells contacted with the SMO inhibitor and a population of cells contacted with the test compound of at least about 1%, 5%, 10%, 20%, 30%, 40%, 50%, 60%, 70%, 80%, 90%, 100%, or more.

Some identified therapeutic compounds can be useful for ameliorating disorders such as a cancer, for example, ovarian cancer. In some embodiments, a population of cells is contacted with a SMO inhibitor, such as BMS-833923 or a compound of Table 3, in combination with a test compound, such as a candidate drug. Contacting a cell with a SMO inhibitor, such as BMS-833923 or a compound of Table 3, in combination with a test agent can include contacting the cell with the SMO inhibitor and the test agent at the same time, at different times, and at overlapping time periods.

Some embodiments also include determining whether the level of cell survival in the population of cells contacted with the SMO inhibitor in combination with the test compound is significantly less than the combined level of cell survival in a population of cells contacted with the SMO inhibitor and a population of cells contacted with the test compound. The significantly lower level of cell survival in the population of cells contacted with the SMO inhibitor in combination with the test compound can include a decrease in the combined level of cell survival of a population of cells contacted with the SMO inhibitor and a population of cells contacted with the test compound of at least about 1%, 5%, 10%, 20%, 30%, 40%, 50%, 60%, 70%, 80%, 90%, 100%, or more.

In some embodiments, a significantly lower level of cell survival in the population of cells contacted with the SMO inhibitor in combination with the test compound compared to the combined level of cell survival in a population of cells contacted with the SMO inhibitor and a population of cells contacted with the test compound is indicative of a therapeutic compound. In some embodiments, each population of cells is mammalian. In some embodiments, each population of cells is human. In some embodiments, each population of cells comprises cancer cells. In some embodiments, each population of cells comprises ovarian cancer cells.

Certain embodiments also include preparing a pharmaceutical composition comprising a test compound which acts in synergy with the SMO inhibitor. In some embodiments, the pharmaceutical composition is suitable for intravenous administration. In some embodiments, the pharmaceutical composition is a pill.

### Kits

Some embodiments include kits comprising a SMO inhibitor, such as BMS-833923 or a compound of Table 3. In some embodiments a kit further comprises a platinum-based chemotherapeutic agent, such as an agent described herein. Agents can include lyophilized compounds. In some such embodiments, the kit can further comprise buffers and carriers for reconstituting the agent. More embodiments include instruments for administering agents to a subject.

While the present invention has been described in some detail for purposes of clarity and understanding, one skilled in the art will appreciate that various changes in form and detail can be made without departing from the true scope of the invention.

### EXAMPLES

### Methods and Materials

Cell Culture

Ovarian cancer cell lines SKOV3, OV90, TOV112D and ES2 were purchased from American Type Culture Collection. Cells were maintained in DMEM/F12 (Invitrogen, Carlsbad, Calif.) supplemented with 1% sodium pyruvate (Invitrogen), 0.2% non-essential amino acids (Invitrogen), and 5% FBS in a humidified atmosphere containing 5% CO₂ at 37° C. Immortalized normal ovarian surface epithelial cell line IOSE80 were grown in a 1:1 combination of two media, Medium 199 (Invitrogen) and MCDB 105 (Cell Applications Inc, San Diego, Calif.) with 10% FBS in a humidified atmosphere containing 5% CO₂ at 37° C. Ovarian cancer cell lines and normal ovarian cell lines were cultured in monolayer in the above stated conditions.

Western Blot Experiments

Nuclear protein extraction was performed as described by Sadowski and Gilman (1993). A confluent monolayer of cells was washed twice in ice-cold PBS. Buffer A (10 mM Hepes, 10 mM KCl, 0.1 mM EDTA, 200 μl of 10% IGEPAL, 1 mM DTT and 10 μl/ml protease inhibitor cocktail (Sigma cat# P8340) was added to the plate and maintained at room temperature for 10 minutes. The lysate was spun at 15,000 g for 3 minutes at 4° C. The supernatant was saved as cytosolic fraction. The pellet was suspended in buffer B (20 mM Hepes, 400 mM NaCl, 1 mM EDTA, 10% glycerol, 1 mM DTT, and 10 μl/ml protease inhibitor cocktail and kept in a shaker for overnight at 4° C. The nuclear lysate was obtained by centrifuging at 15,000 g for 5 minutes at 4° C.

A confluent monolayer of cells was washed once with ice cold PBS. PBS was aspirated and 0.5 ml of ice cold NP40 lysis buffer (1% NP40, 150 mM NaCl, 50 mM Tris-base, and protease inhibitor cocktail 10 μl/ml) was added to the plate. Cells were scraped off and transferred to a 1.5 ml eppendorf tube. The tube was placed on ice to ensure complete cell lysis for 1 hour. Cells were centrifuged at 14,000 g for 10 minutes and the supernatant was collected as the whole cell lysate.

Protein concentration was measured by using Precision Red protein assay reagent (Cytoskeleton, cat# ADV02-A). 30 μg proteins were subjected to SDS-PAGE and transferred to polyvinylidene fluoride membrane (0.2 μm). The membranes were blocked with 5% nonfat dry milk in Tris-buffered-saline Tween 20 (TBST) [1 M Tris (pH 7.3), 9% NaCl and 0.05% Tween-20] and incubated with primary antibodies overnight at 4° C. After washes with TBST (pH 7.3) and incubation with the respective horseradish peroxidase tagged secondary antibody, the blots were developed using SuperSignal (Pierce, Rockford, Ill., USA). The Gli1 antibody (Santacruz Biotech, SantaCruz, Calif.) was used at a final dilution of 1:100 and the SMO antibody (LifeSpan Biosciences, Inc., Seattle, Wash.) and PTCH antibody (Santacruz Biotech) were used at a final concentration of 1:200.

IC50 Dosage Calculations with SMO Inhibitor BMS-833923

Ovarian cancer cells were seeded at a density of 4,000 cells/well in 96 well plates. The following day, fresh medium was added to each well and they were treated with BMS-833923 at a dose-escalation at the following doses 0, 0.1, 0.25, 0.5, 1, 2, 5, 10, and 20 μM. These cells were kept at 37° C. in a humidified 5% CO₂ atmosphere. Every 24 hours, 20 μl/well of MTS reagent were added in 96 well plates and stored in 37° C. incubator for at least 3 hours followed by absorbance reading at 490 nm. The assay was carried out up to 72 hrs to obtain the IC50 concentrations of each agent.

Quantitative Real-Time PCR

Total RNA was isolated using Trizol reagent from ovarian cancer cell line monolayer and spheroids-forming cells according to the manufacturer instructions (Invitrogen Corporation, Carlsbad, Calif.) and cDNA was prepared using High Capacity Reverse Transcriptase Kit (Applied Biosystem, Foster City, Calif.). Real-time PCR was performed using a Bio-Rad iQ5 Real-time Detection system (Bio-Rad). All reactions were done as three independent replicates. All assays were done using the TaqMan Gene Expression Assays from Applied Biosystems. Primers and probes for the TaqMan system were selected from the Applied Biosystems website. [Gli1 assay ID: Hs01110766 ml, Patched (PTCH) assay ID: Hs00970980 ml. The relative expression mRNA levels of GLI1, and PTCH were normalized to internal control glyceraldehyde-3-phosphate dehydrogenase (assay ID: Hs99999905 ml) levels (2^{-δδCT}).

Localization of Gli1 by Immunofluorescent Staining

BMS-833923, a Hh signaling inhibitor, has previously demonstrated antitumor properties via direct binding to SMO. Considering intranuclear Gli1 confers an activated Hh pathway, SKOV3 cells were evaluated for Gli1 location after BMS-833923 treatment and compared to control (no treatment). Cells were grown on Poly-L-Lysine coated Glass Coverslips (BD Bioscience) for overnight, then incubated with 5 μM BMS-833923 for 24 hours, respectively, fixed in 4% paraformaldehyde for 15 minutes at room temperature, washed with PBS and permeabilized with 0.25% Triton X-100 (Sigma) for 10 minutes. Primary antibody (Anti-human Gli1 antibody, Santa Cruz Biotechnology, Inc.) incubation followed for overnight at 4° C. After washing thoroughly with PBS, cells were further probed with secondary antibody (anti-rabbit IgG-FITC, Sigma) for one hour at room temperature. Nuclei were counterstained with diluted DAPI (Sigma) for 3 minutes before mounting and analyzing with microscope.

Matrigel Invasion Assay

Fluorescence-activated cell sorting (FACS) was performed in order to sort into 2 distinct phenotypes: 1) ovarian cancer stem cell phenotype (OCSC=CD44+/CD24−) and 2) non-ovarian cancer stem cells (non-OCSC=all other phenotypes). Each ovarian cancer phenotype was evaluated for invasion properties using the Matrigel invasion assay for the SKOV3 and OV90 cell lines. Matrigel™-coated chamber (BD Pharmingen) were rehydrated for 2 hours in humidified tissue culture incubator at 37° C., 5% CO₂. After rehydration, the medium was carefully removed without disturbing the layer of Matrigel Matrix on the membrane. The cell suspension was prepared in serum-free medium containing 10⁵ cells/mL. The lower chambers of the 24-well plate were filled with 750 μL serum-free medium containing 10 μg/mL fibronectin as a chemotactic factor. 500 μL serum-free medium containing 5×10⁴ cells were added to the upper chambers. The plate was incubated in a humidified tissue culture incubator at 37° C., 5% CO₂ atmosphere for 24 hours. The cells were then fixed, stained and counted. Numbers of invaded cells at least 10 consecutive fields were enumerated and their average was calculated. Data are expressed as the number of migrating cells per field. Three groups were evaluated: 1) non-OCSC; 2) OCSC; 3) OCSC plus BMS833923 treatment.

Cell Proliferation Assay with BMS-833923 and with or without Chemotherapy

An MTS in vitro assay was performed using chemotherapy agents commonly used for ovarian cancer (carboplatin, paclitaxel, gemcitabine, topotecan and liposomal doxorubicin) with or without BMS-833923. Ovarian cancer cells ES2, SKOV3, OV90 and TOV112D were seeded at a density of 4,000 cells/well in 96-well tissue culture plates. The following day, fresh medium was added to each well and treated with IC50 concentrations of BMS-833923 as well as the aforementioned chemotherapies and kept at 37° C. in a humidified 5% CO₂ atmosphere. Every 24 hr, 20 μl/well of MTS reagent were added in 96 well plates and stored in 37° C. incubator for about 3 hr followed by absorbance reading at 490 nm. The assay was carried out up to 72 hrs at various concentrations described herein.

Detecting Synergy of Chemotherapy Drug Combinations Using Calculsyn Software

With a cross-diagonal multidrug treatment design, the combination index (CI) was calculated to determine mathematical synergy using the median effect method per Calcusyn software (Biosoft, Ferguson, Mo.). This software utilizes multiple drug dose-effect calculations using the Median Effect methods (Chou T. C., et al., Trends Pharmacol. Sci. 4, 450-454). Varying percentages of IC50 doses (25%, 50%, 75%, 100%, and 125%) for each agent in a typical cross-diagonal treatment design is depicted in Table 4. The fraction of cells affected (Fa) by carboplatin and BMS-833923 was used to calculate dose response curves and the CI. Statistically Calcusyn quantifies phenomena such as synergism vs. additive effects as well as inhibition (antagonism). Calcusyn software defines synergy as a CI value<1 with the extent of synergism stratified as follows: Possible Synergy: 1.0-0.91; Moderate Synergy: 0.9-0.85; and Clear Synergy: 0.7-0.3. A CI=1 may be indicative of additivity; a CI>1 may be indicative of antagonism.

TABLE 4

| | | BMS-899923 IC50 dose | | | | |
|---|---|---|---|---|---|---|
| | | 25% | 50% | 75% | 100% | 125% |
| Carboplatin | 25% | X | | | | |
| IC50 dose | 50% | | X | | | |
| | 75% | | | X | | |
| | 100% | | | | X | |
| | 125% | | | | | X |

Statistical Analysis

For comparative effectiveness of experiments, student's t-test and ANOVA were utilized for continuous variables where appropriate. Statistical significance is determined at p<0.05.

Xenograft Experiments

Female SCID mice 6-8 weeks old were inoculated i.p. with 5×10⁶ A2780 or A2780/CP70 cells suspended in 200 μL PBS (PH 7.4). Seven days later when the tumor reached a size of greater than 150 mm³, the mice were randomly divided into 4 groups (8 mice per group) as follows: (1) PBS-treated control; (2) BMS-833923 alone (100 mg/kg, i.p. daily, two weeks); (3) Carboplatin alone (40 mg/kg, i.p. twice a week, two weeks); (4) Combination of BMS-833923 and Carboplatin treatment. Mice were weighed and tumor diameters were measured with calipers every 2-3 days for 20 days. The tumor volumes (mm³) were calculated as (W²×L)/2, where W and L are the minor and major diameters (in millimeters), respectively.

### Example 1

### Ovarian Cancer Cells Demonstrate Enhanced Activation of Hh Pathway

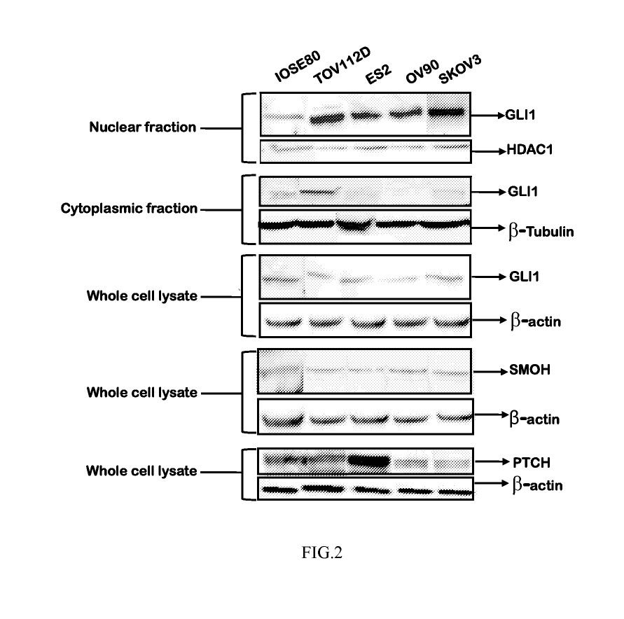

Western blot analysis was performed as described above. Activation of the Hh pathway is initiated by the cell surface protein, smoothened (SMO) which leads to the translocation of cytoplasmic Gli1 to the nucleus to function as a transcription factor. As such, intranuclear Gli1 was detected by Western blot in nuclear fraction from all cell lines evaluated with increased expression in ovarian cancer cell lines (ES2, SKOV3 and TOV112D) compared to immortalized normal ovarian cell line (IOSE80) (FIG. 2). Consistent with a normally dormant innate pathway, all cell lines expressed varying levels of SMO and PTCH.

### Example 2

### IC50 Of BMS-833923 was Calculated for Ovarian Cancer Cell Lines

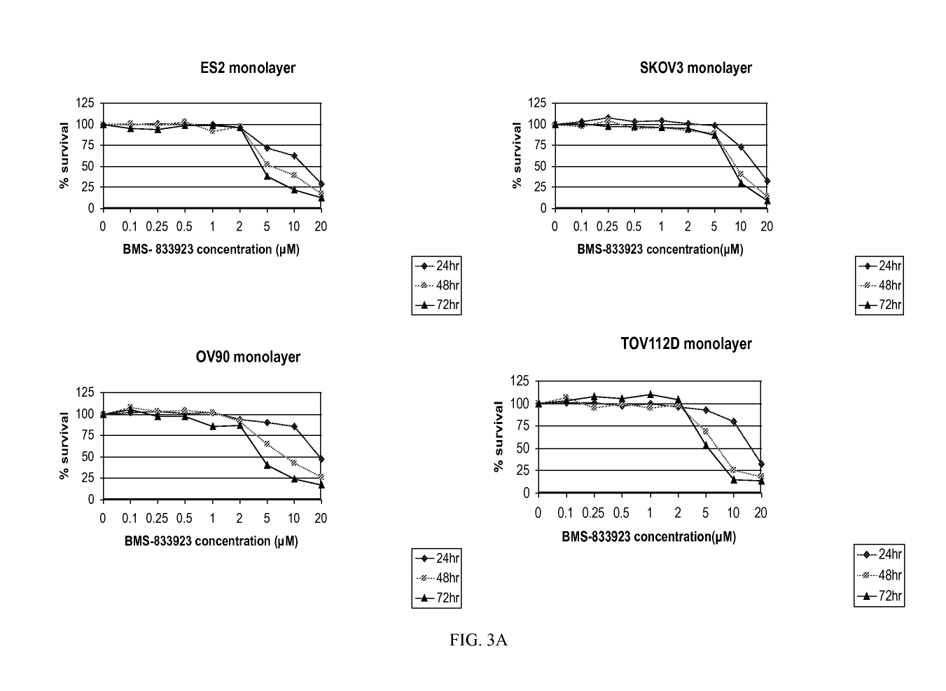

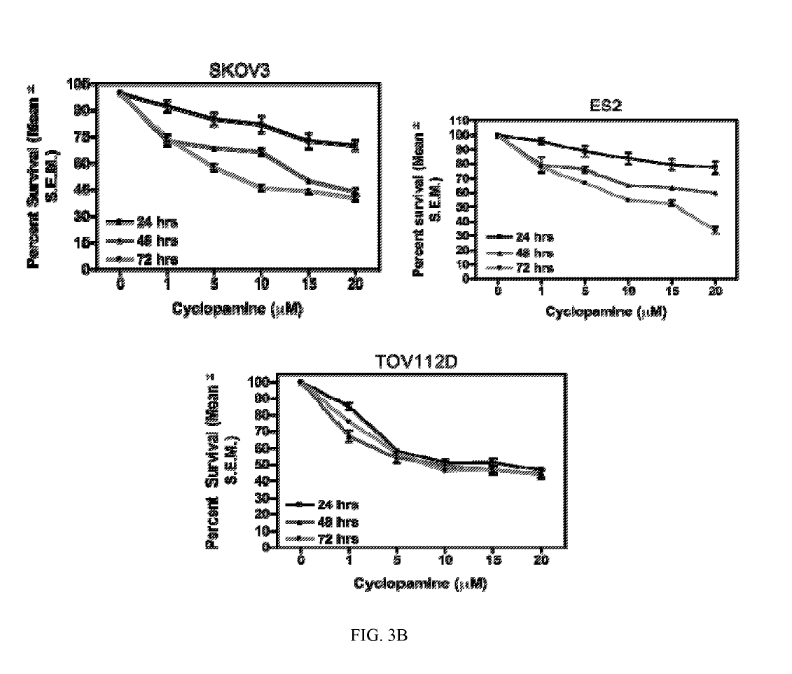

IC50 dosage determinations were performed as described above. BMS-833923 was given in a dose-escalation fashion and demonstrated typical dose-response curves with IC50 between 5-10 μM for the treated ovarian cancer cell lines in monolayer (ES2=4 μM; TOV112D=5 μM; OV90=4 μM; SKOV3=7.5 μM) (FIG. 3A). BMS-833923 IC50 doses were used for all related experiments for specific ovarian cancer cell lines.

### Example 3

### BMS-833923 Treatment Down-Regulates Hedgehog Pathway Mediators

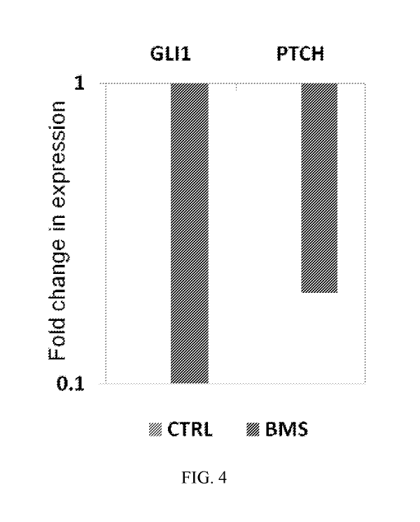

Quantitative realtime PCR (RT-PCR) was performed as described above. To assess the effects of inhibition of SMO by BMS-833923, levels of GLI1 and PTCH were measured by quantitative RT-PCR. SKOV3 ovarian cancer cell line was treated with 2.5 μM BMS-833923 for 24 hr and 48 hr and compared to untreated controls. Utilizing an untreated baseline of 1 on logarithmic scale, RTQ of GLI1 demonstrated a 10-fold decrease in activity with BMS-833923 treatment (FIG. 4). Additionally, PTCH also demonstrated a down-regulation in activity with BMS-833923 treatment compared to untreated controls.

### Example 4

### BMS-833923 Inhibits Cancer Invasion Properties of Ovarian Cancer Stem Cells

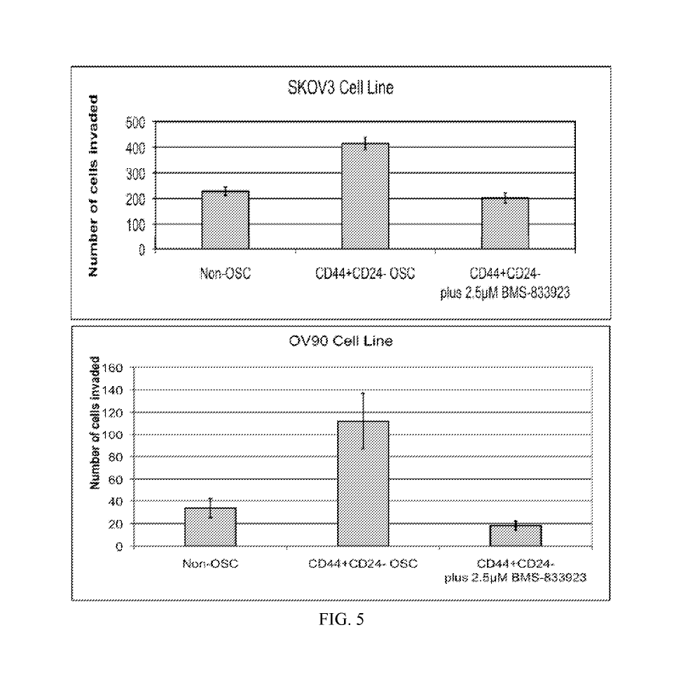

One aggressive property of cancer includes the ability to invade tissues locally. Therefore, the ability of Ovarian Cancer Stem Cells (OCSC) with CD44+/CD24− phenotype to invade via Matrigel invasion assay was evaluated. Matrigel invasion assays were performed as described above. SKOV3 and OV90 cell lines were sorted via FACS into an OCSC and non-OCSC populations and plated for invasion. Compared to non-OCSC, OCSC (CD44+/CD24−) demonstrated a 1.8-fold increase in invasive properties in SKOV3 cell line and 3.3-fold increase in invasion for OV90. (p<0.001 for each) (FIG. 5). Of note, BMS-833923 treatment of OCSC (CD44+/CD24−) rendered the invasive properties of each cell line to a value lower than non-OCSCs.

### Example 5

### BMS-833923 Inhibits Activation of Hh Pathway by Preventing Intranuclear Translocation of Gli1

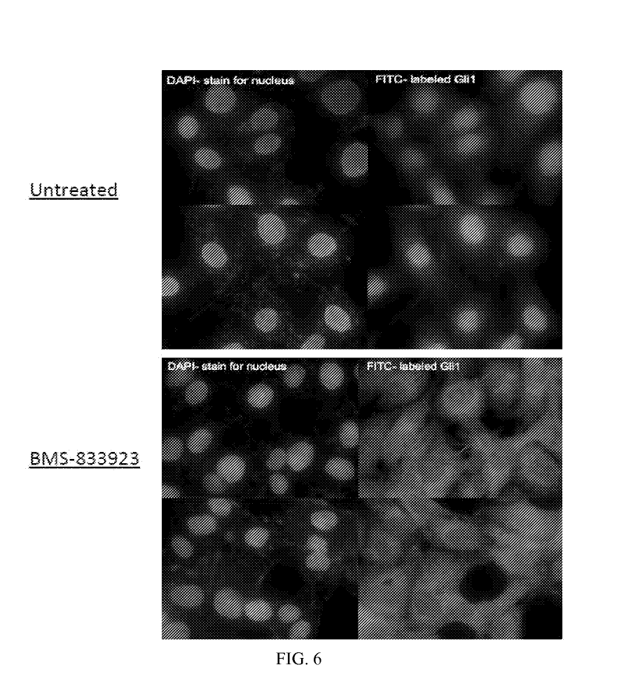

SKOV3 cells were treated with 2.5 μBMS-833923 and the cellular location of Gli1 determined at various times using immunofluorescence. Immunofluorescence analysis was performed as described above. In untreated control cell, Gli1 is concentrated in the nucleus at 24 hrs, indicating an activated Hh pathway. Conversely, SKOV3 ovarian cancer cells treated with IC50 concentration of BMS-833923 for 24 hours resulted in Gli1 being excluded from the nucleus with evidence of nuclear “shadowing” and vacuolization (FIG. 6). Both indicating restriction of Gli1 from the nucleus and inhibition of Hh pathway.

### Example 6

### Combination of Chemotherapy and BMS-833923 was More Effective than Chemotherapy or BMS-833923 Alone

Combination indices were calculated to determine mathematical synergy using the median effect method per Calcusyn software (Biosoft, Ferguson, Mo.) as described above. Monolayers of SKOV3 cells were contacted with various chemotherapeutic agents, and BMS-833923 in combination with various chemotherapeutic agents. The chemotherapeutic agents included Carboplatin, Taxol™, Gemzar™, Topotecan (Hycamtim™), and Doxil™. The percentage survival of the contacted SKOV3 cells was measured over 72 hours. Synergistic effects were calculated statistically using Calcusyn™ Software. A combination index (CI) less than 1 indicates synergy; a CI equal to 1 indicates additivity; a CI greater than 1 indicated antagonism.

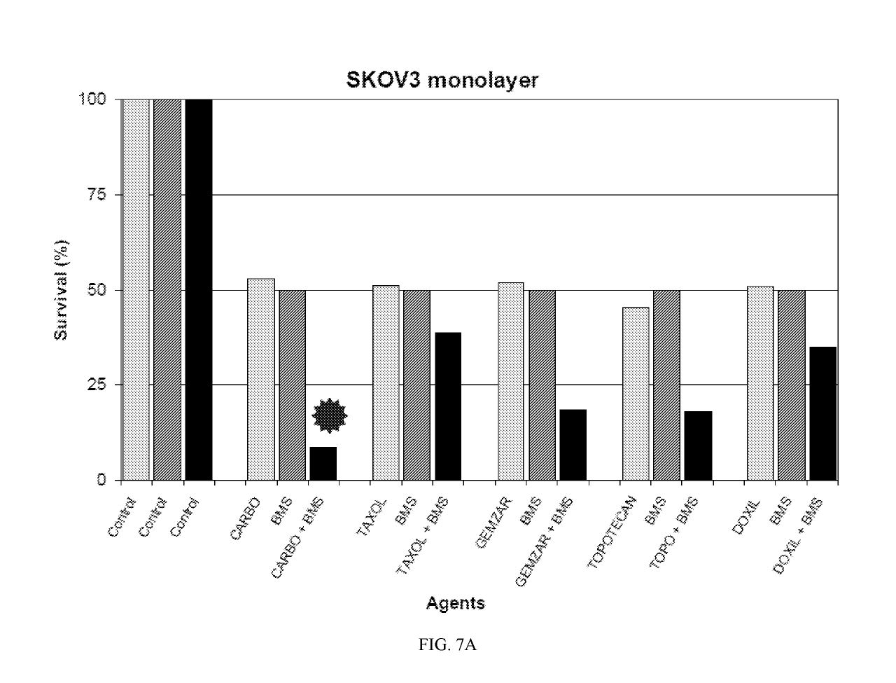

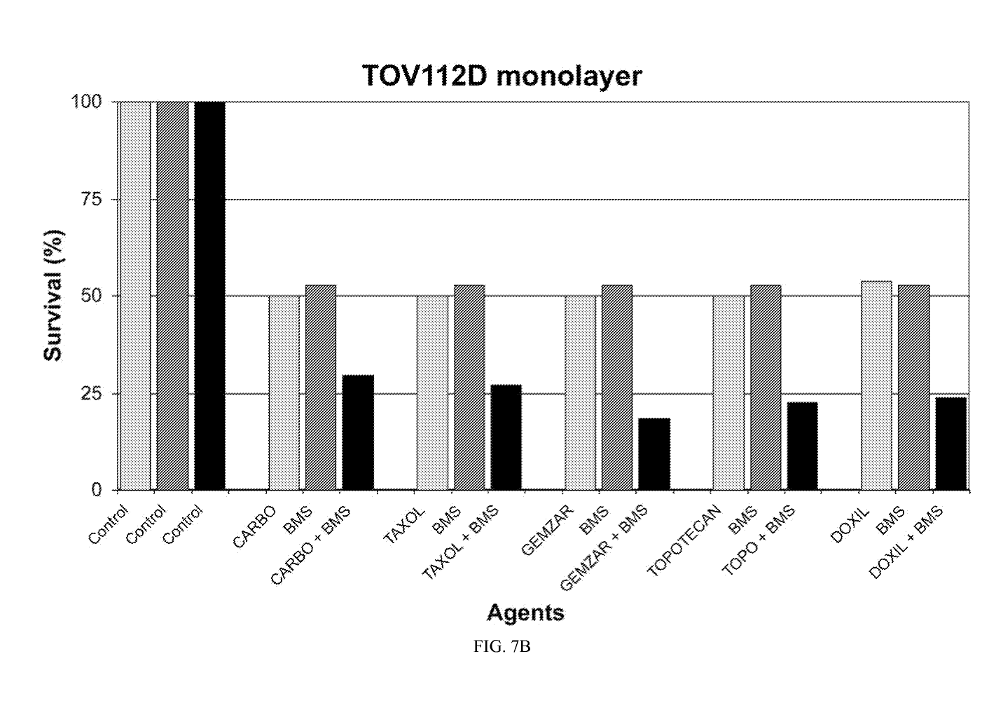

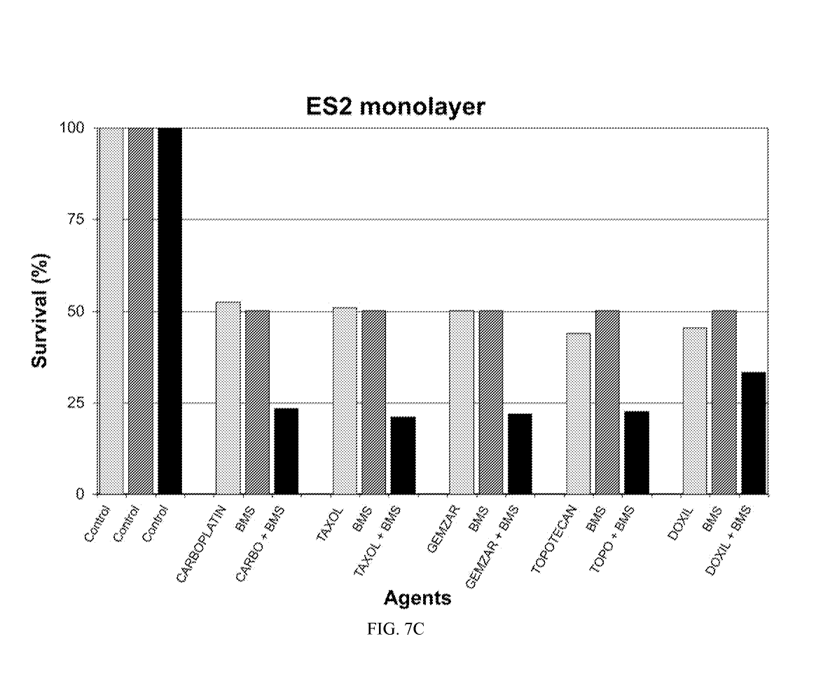

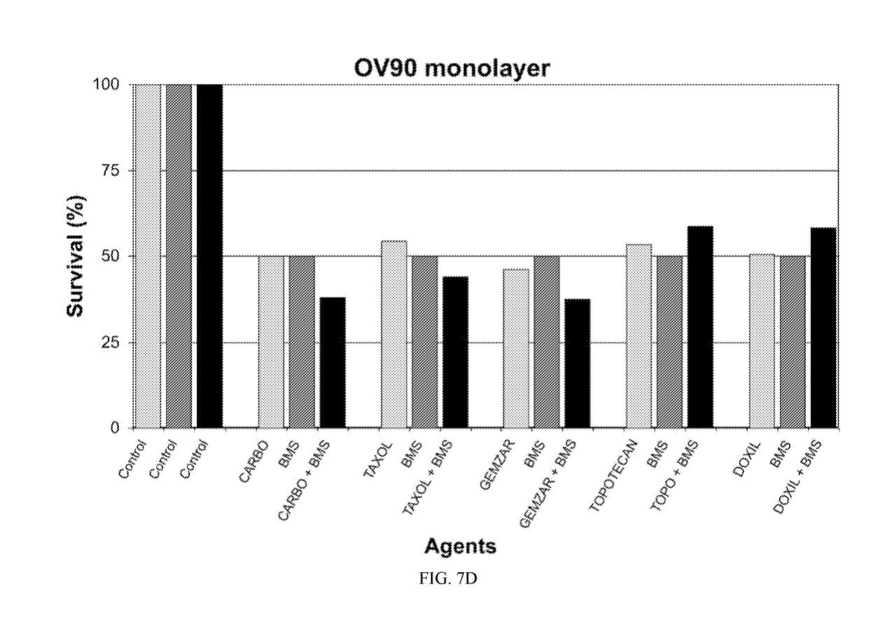

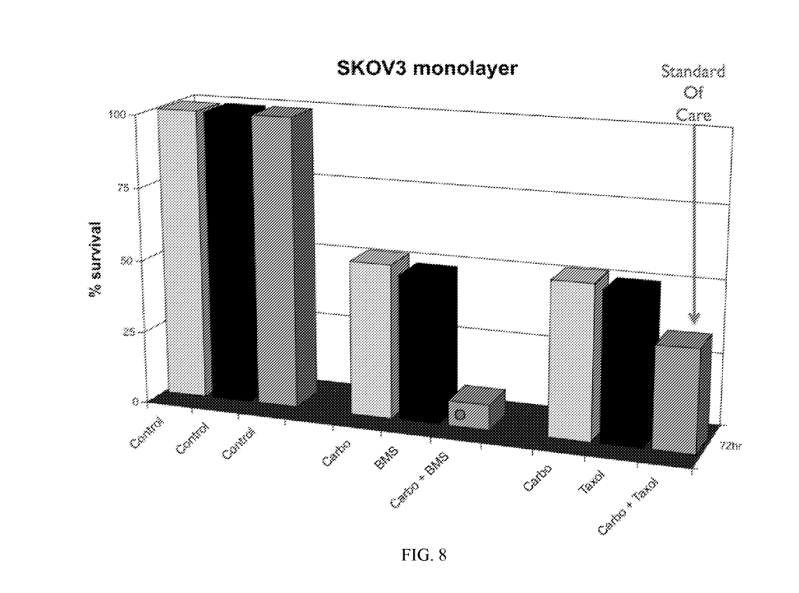

In MTS cell survival assays, the combination of chemotherapy and BMS-833923 was more effective than chemotherapy or BMS-833923 alone (FIGS. 7A-7D). For combinational therapeutic assays utilizing IC50 concentrations, any cell survival of <25% is suggestive of a possible synergy. Although several combinations were suggestive, the greatest combinational effect was demonstrated with the combination of BMS-833923 and carboplatin (8% cell survival). Indeed, this combination was so effective, it demonstrated a greater efficacy than the current standard of care of combination chemotherapy regimen of carboplatin and paclitaxel (FIG. 8).

### Example 7

### BMS-833923 Plus Carboplatin Demonstrates Synergistic Effect on Ovarian Cancer

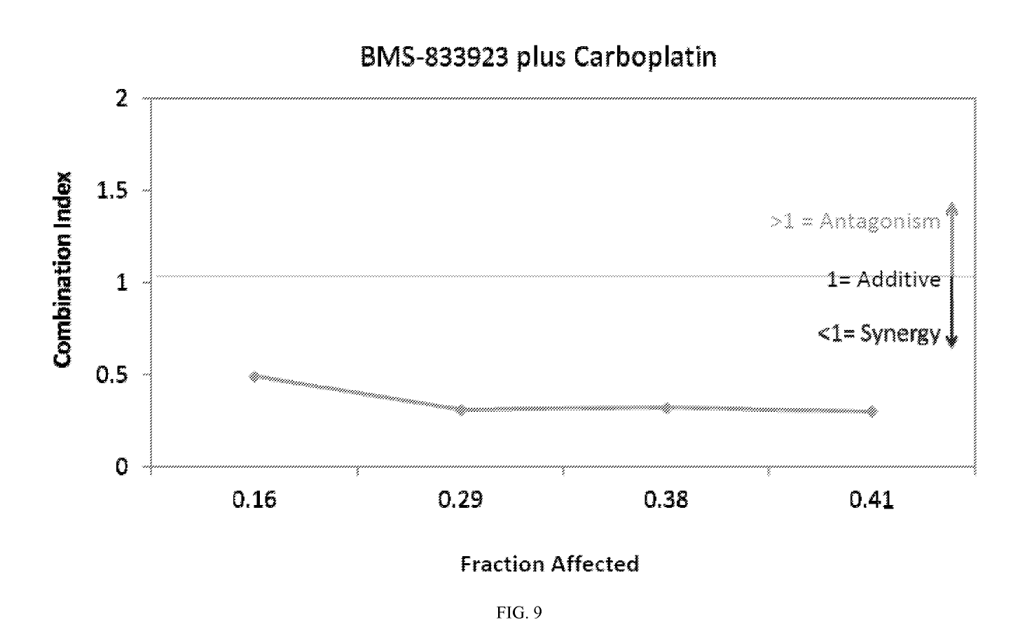

Combination indices were calculated to determine mathematical synergy using the median effect method per Calcusyn software (Biosoft, Ferguson, Mo.) as described above. Significant cell death was demonstrated with the combination of BMS-833923 and carboplatin (8% cell survival) which is highly suggestive of a combination synergy. Calcusyn CI calculations determined that multiple dosage combinations resulted in synergy with a CI range of 0.3 to 0.49. Per prior definitions this represents Clear Synergy defined as CI<0.7. This level of synergy would result in a 2- to 14-fold reduction of carboplatin dose required with the addition of BMS-833923. (Table 5 and FIG. 9).

TABLE 5

| BMS-833923 | | Fraction | | |
|---|---|---|---|---|
| (μM) | Carboplatin (μM) | affected (%) | CI | DRI |
| 4 | 60 | 16.6 | 0.49 | 14 |
| 4 | 70 | 29.1 | 0.31 | 8 |
| 5 | 60 | 38.4 | 0.32 | 2 |
| 5 | 70 | 41.8 | 0.30 | 2 |

The dose reduction index (DRI) was determined for experimental values using the Calcusyn software package, with the relative CI and the fraction affected to determine the relative reduction in a dose to achieve similar effects with a single agent (Table 6).

TABLE 6

| | BMS- | | | |
|---|---|---|---|---|
| Fraction | 833923 | Carboplatin | BMS-833923 | Carboplatin |
| affected (%) | (μM) | (μM) | DRI | DRI |
| 16.6 | 9.2573 | 892.1349 | 2.314 | 14.869 |
| 29.1 | 12.8916 | 5.642e+008 | 3.223 | 8.06e+006 |
| 38.4 | 15.6024 | 1.243e+012 | 3.120 | 2.07e+010 |
| 41.8 | 16.6516 | 1.716e+013 | 3.330 | 2.45e+011 |

### Example 8

### Carboplatin Treatment Sensitizes Ovarian Cancer Cells to BMS-833923 and Increases Overall Cytotoxic Effect

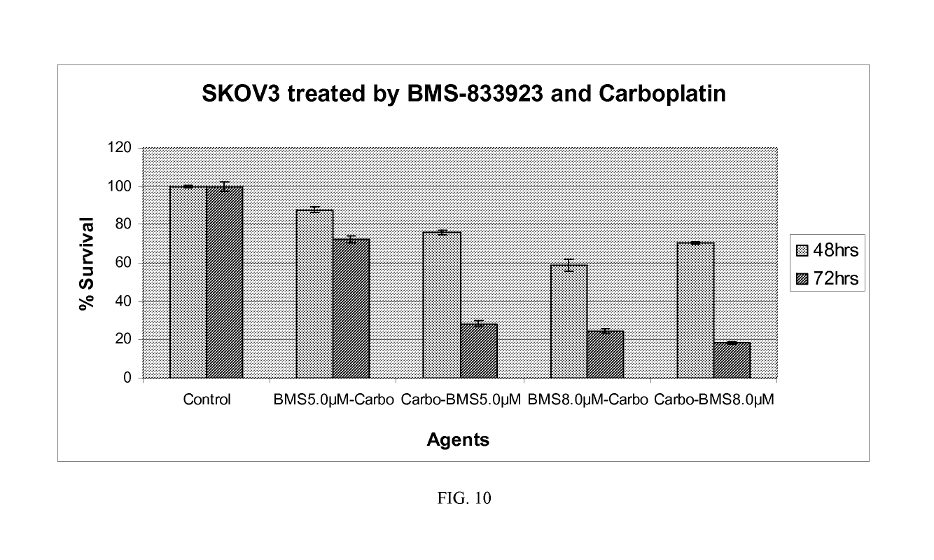

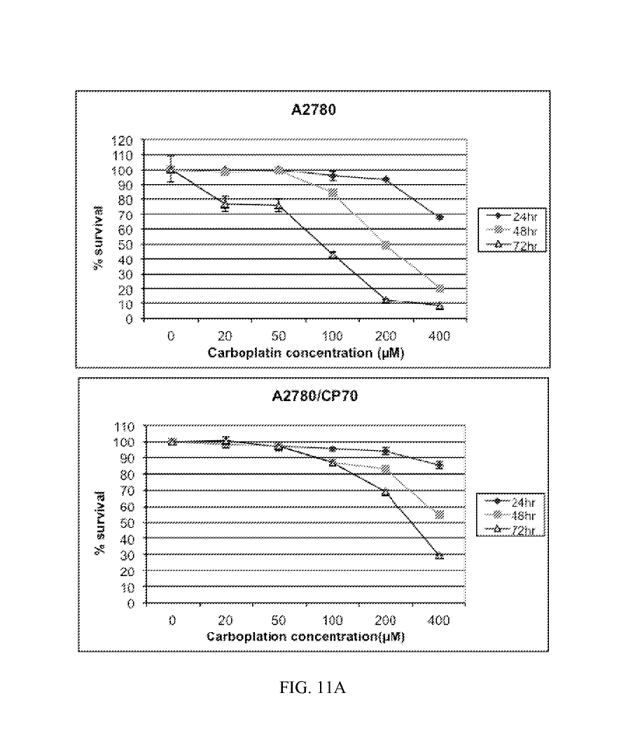

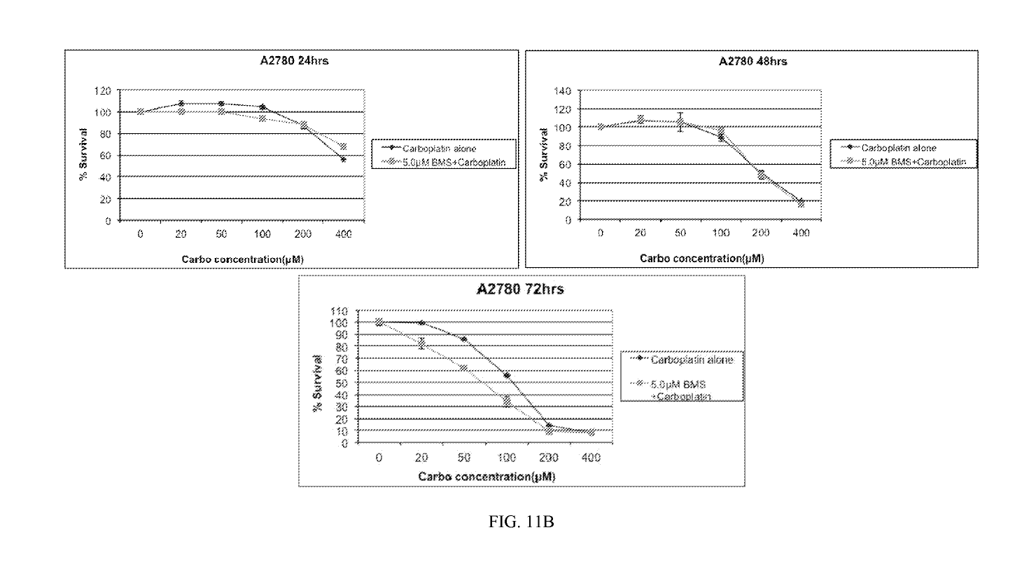

Considering the synergy determined with carboplatin and BMS-833923, the effect of sequential delivery of agents was determined. In sequential order, deliveries of carboplatin 1^{st}—followed by 5.0 μM or 8.0 μM BMS-833923; or 5.0 μM or 8.0 μM BMS-833923 1^{st}—followed by Carboplatin were determined. For each group, the first agent was given at timepoint 0 hours with the second agent given 24 hours later. Then using MTS assay the cell death at time points 48 and 72 hours was determined (FIG. 10). Compared to controls in SKOV3 cell lines at 100% cell survival, BMS 1^{st} group showed a 79% cell survival at 72 hours of total treatment, while the Carbo 1^{st} group demonstrating a 20% cell survival at 72 hours. Treating cells with Carboplatin at 0 hr and BMS-833923 at 24 hr had the greatest killing effect on the cells.

### Example 9

### BMS-833923 Reverses Platinum Resistance in Ovarian Cancer Cells

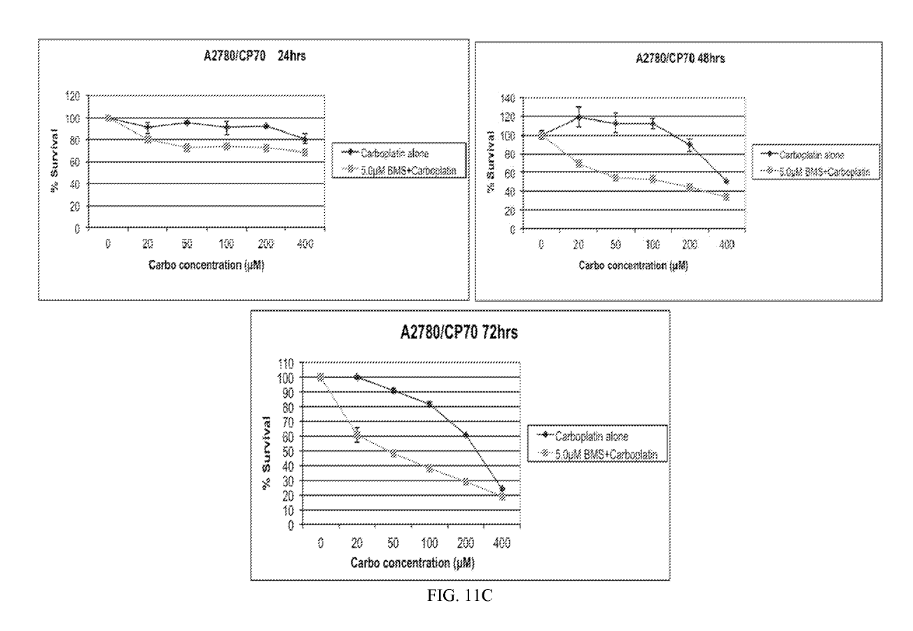

Considering the synergy described above and that the Hedgehog pathway is associated with cancer stem cell properties. In the effect of BMS-833923 plus carboplatin in two isogenic ovarian cancer cell lines specifically designed to evaluate platinum resistance mechanisms: A2780 (platinum sensitive) & A2780/CP70 (platinum resistant). Each cell line was evaluated to determine the IC50 dose of carboplatin to ensure a discernable difference in platinum treatment could be determined (FIG. 11A). The A2780/CP70 cell line required a 4-fold increase in carboplatin to reach IC50 (A2780/CP70=400 μM; A2780=100 μM). The addition of BMS-833923 at 5 μM demonstrated a 10-fold reduction of platinum needed to achieve the same cell death in A2780/CP70 platinum resistant cell line. (FIG. 11B and FIG. 11C).

### Example 10

### In Vivo Assessment of BMS-833923 and Carboplatin as Single Agents and as Combination Therapy in the Carboplatin Sensitive A2780 and Carboplatin Resistant CP2780 Tumor Models

Xenograft experiments were performed as described above. Carboplatin was dosed days 7, 11, 14, 18; BMS-833923 was dosed days 8 through 20. The experiment was terminated due to excessive tumor size in vehicle and BMS-833923 treatment groups.

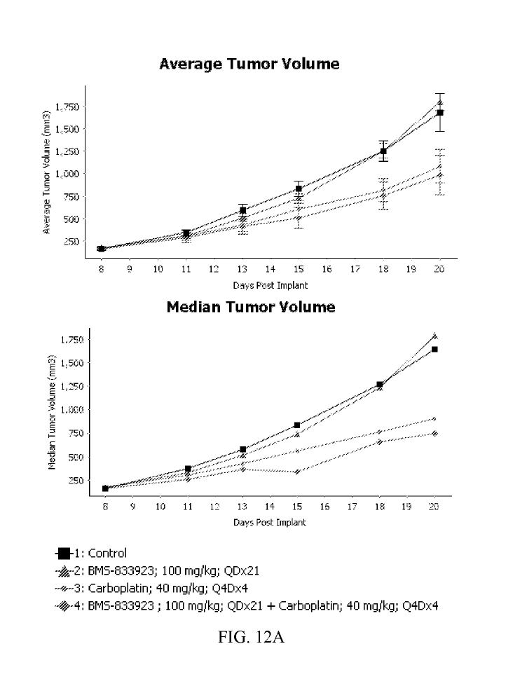

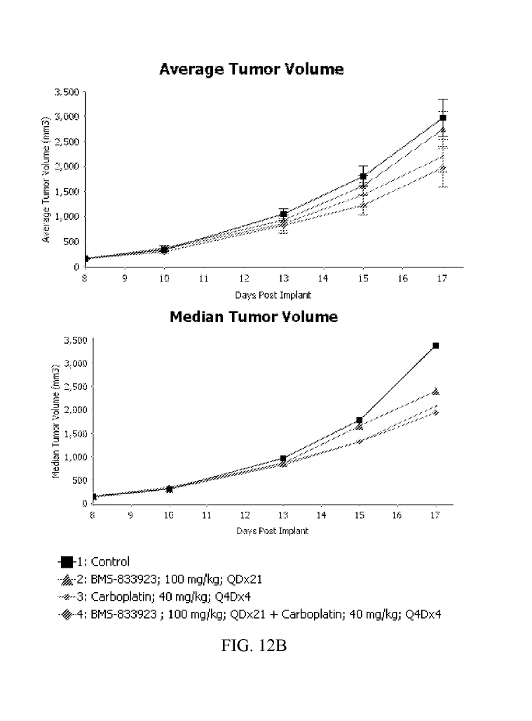

Previously, 100 mg/kg BMS-833923+40 mg/kg carboplatin has been demonstrated to represent the MTD for combination therapy. Treatment with BMS-833923 appeared to have little effect on the growth of A2780 xenografts in vivo. Carboplatin treatment inhibited growth of A2780 tumor xenografts (FIG. 12A).

The CP2780 was derived from the A2780 Ovarian tumor xenograft model by selection for resistance to cisplatin. The CP2780 growth appeared to be more robust than observed for parental A2780 model (FIG. 12B). Average tumor volume doubling time for CP2780 was estimated at 2 days. Doubling time for parental A2780 tumor model was estimated at 3 days. Treatment with BMS-833923, Carboplatin, or both, appears to have little effect on the growth of CP2780 xenografts in vivo.

### Example 11

### Percent Body Weight Change Associated with Single Agent and Combination Therapy with BMS-833923 and Carboplatin

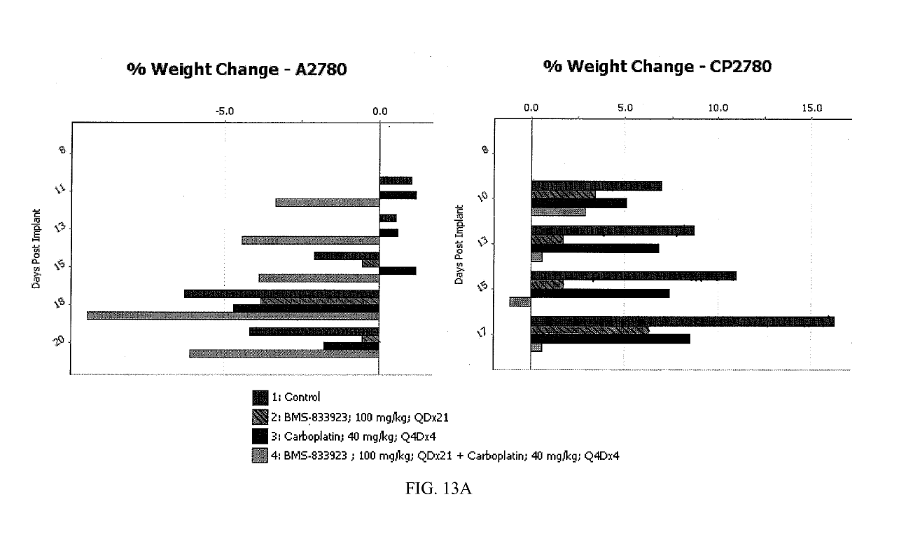

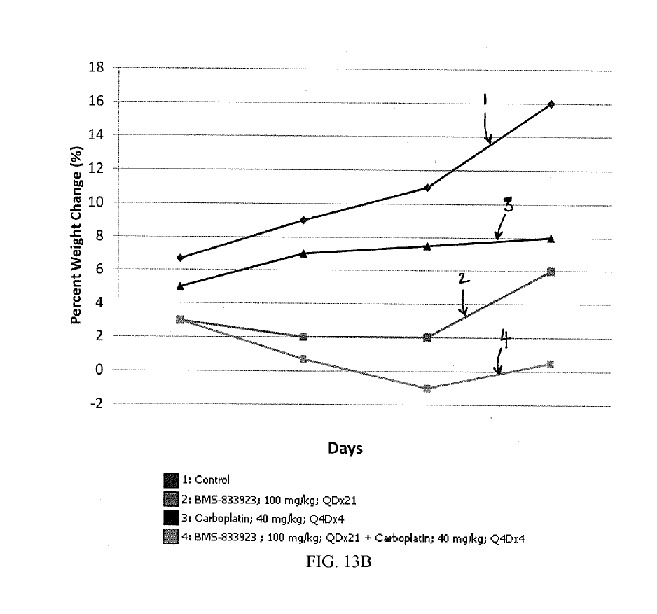

Xenograft experiments were performed as described above. Percent body weight change of the combination treatment group with BMS-833923 and Carboplatin was much less than other groups (Combination<Carboplatin alone<BMS-833923 alone<PBS-treated control) (FIG. 13A and FIG. 13B). However, under such dosage (100 mg/kg, i.p. daily, for two weeks), treatment with BMS-833923 appeared to have little effect on the growth of A2780 or A2780/CP70 xenografts in vivo (FIG. 13). As expected, treatment with Carboplatin (40 mg/kg, i.p. twice a week, two weeks) inhibited growth of A2780 tumor xenografts. However, this dosage has little effect on the growth of CP2780 xenografts in vivo.

### Example 12

### Upregulation of SHH in Carboplatin Treated Cells

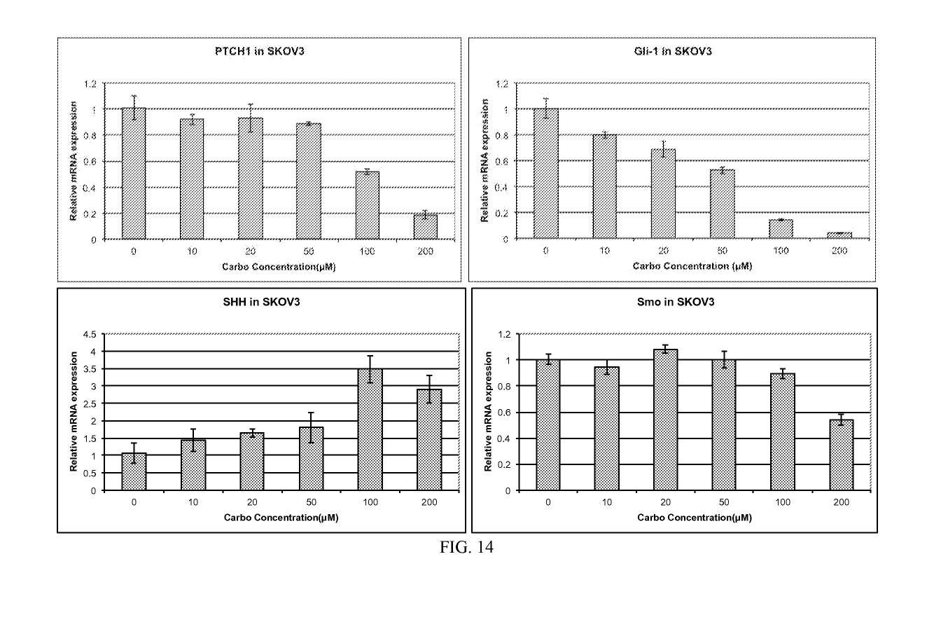

SKOV3 cells were treated with various concentrations of carboplatin and the levels of SHH, Smo, PTCH, and Gli1 determined using quantitative real time PCR. Carboplatin treatment unregulated SHH (FIG. 14). This suggested that carboplatin may sensitize SKOV3 cells to SMO inhibition by upregulating SHH.

One strategy to improve success of ovarian cancer therapy is to enhance its sensitivity to platinum agents. If chemoresistance could be overcome, response rates, overall survival and cure rates would significantly improve. As described herein, inhibiting the hedgehog pathway improved the sensitivity of platinum-resistant ovarian cancer to platinum agents up to 10-fold. As such, this is a discovery that could significantly enhance the effectiveness in the treatment of patients with ovarian cancer.

In summary, the importance of platinum-sensitivity to ovarian cancer survival is well established. Ovarian cancer stem cells and specifically the hedgehog pathway contribute to resistance to platinum-based therapy has been shown. Considering that reversing platinum-resistance and/or sensitizing ovarian cancer to platinum agents can directly improve patient outcomes these findings are vitally important and could be paramount to the overall prognosis of this deadly disease. A plethora of data exists which demonstrates that platinum sensitivity confers: greater response rates; lower recurrence rates; longer disease free intervals; longer survivals, and more cures. Additionally, this could offer treatment options to a group of ovarian cancer patients with the worst prognosis and could reduce doses typically used with platinum agents and thereby reduce systemic side effects and toxicities.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-288. (canceled)

289. A method of killing or retarding the growth of a neoplastic cell comprising: contacting the cell with an effective amount of a SMO inhibitor in combination with an effective amount of a chemotherapeutic agent.

290. The method of claim 289, wherein the chemotherapeutic agent comprises a platinum-based chemotherapeutic agent.

291. The method of claim 289, wherein the effective amount of the chemotherapeutic compound is at least about 10% less than the effective amount of the chemotherapeutic compound in the absence of the SMO inhibitor.

292. The method of claim 289, wherein the effective amount of the SMO inhibitor is an amount which significantly reduces the IC50 of the chemotherapeutic compound.

293. The method of claim 289, wherein the effective amount of the chemotherapeutic compound and the effective amount of the SMO inhibitor have a Combination Index less than 1 as determined by Calcusyn software.

294. The method of claim 289, wherein the SMO inhibitor is selected from a compound of Table 3.

295. The method of claim 289, wherein the SMO inhibitor comprises BMS-833923, having the structure:

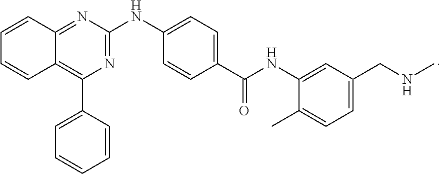 [SMILES: CNCc1ccc(C)c(NC(=O)c2ccc(Nc3nc(-c4ccccc4)c4ccccc4n3)cc2)c1]

296. The method of claim 295, wherein the cell is contacted with the BMS-833923 and the chemotherapeutic agent sequentially.

297. The method of claim 295, wherein contacting the cell with the chemotherapeutic agent commences before contacting the cell with the BMS-833923.

298. The method of claim 295, wherein contacting the cell with the chemotherapeutic agent is before contacting the cell with the BMS-833923.

299. The method of claim 295, wherein contacting the cell with the chemotherapeutic agent is less than 48 hours prior to contacting the cell with the BMS-833923.

300. The method of claim 295, wherein contacting the cell with the chemotherapeutic agent commences after contacting the cell with the BMS-833923.

301. The method of claim 289, wherein the chemotherapeutic agent is selected from the group consisting of cisplatin, carboplatin, nedaplatin, oxaliplatin, satraplatin, triplatin tetranitrate, taxol, gemcitabine, topotecan hydrochloride, doxorubicin and pegylated doxorubicin.

302. The method of claim 289, wherein the cell comprises a platinum resistant ovarian cancer cell.

303. The method of claim 289, wherein the cell comprises an ovarian cell.

304. The method of claim 289, wherein the cell comprises an ovarian cancer stem cell.

305. A method of increasing the sensitivity of a neoplastic cell to a chemotherapeutic compound comprising contacting the cell with an effective amount of a SMO inhibitor and an effective amount of the chemotherapeutic compound, wherein the effective amount of the chemotherapeutic compound is significantly less than the effective amount of the chemotherapeutic compound in the absence of the SMO inhibitor.

306. The method of claim 305, wherein the chemotherapeutic agent comprises a platinum-based chemotherapeutic agent.

307. The method of claim 305, wherein the effective amount of the chemotherapeutic compound is at least about 10% less than the effective amount of the chemotherapeutic compound in the absence of the SMO inhibitor.

308. The method of claim 305, wherein the effective amount of the SMO inhibitor is an amount which significantly reduces the IC50 of the chemotherapeutic compound.

309. The method of claim 305, wherein the effective amount of the chemotherapeutic compound and the effective amount of the SMO inhibitor have a Combination Index less than 1 as determined by Calcusyn software.

310. The method of claim 305, wherein the SMO inhibitor is selected from a compound of Table 3.

311. The method of claim 305, wherein the SMO inhibitor comprises BMS-833923, having the structure:

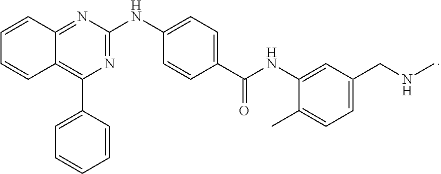 [SMILES: CNCc1ccc(C)c(NC(=O)c2ccc(Nc3nc(-c4ccccc4)c4ccccc4n3)cc2)c1]

312. The method of claim 305, wherein the chemotherapeutic agent is selected from the group consisting of cisplatin, carboplatin, nedaplatin, oxaliplatin, satraplatin, triplatin tetranitrate, taxol, gemcitabine, topotecan hydrochloride, doxorubicin and pegylated doxorubicin.

313. The method of claim 305, wherein the cell comprises a platinum resistant ovarian cancer cell.

314. The method of claim 305, wherein the cell comprises an ovarian cell.

315. The method of claim 305, wherein the cell comprises an ovarian cancer stem cell.

316. A method of ameliorating cancer in a subject comprising: administering to the subject an effective amount of a SMO inhibitor in combination with an effective amount of a chemotherapeutic agent.

317. The method of claim 316, wherein the chemotherapeutic agent comprises a platinum-based chemotherapeutic agent.

318. The method of claim 316, wherein the effective amount of the chemotherapeutic compound is at least about 10% less than the effective amount of the chemotherapeutic compound in the absence of the SMO inhibitor.

319. The method of claim 316, wherein the effective amount of the SMO inhibitor is an amount which significantly reduces the IC50 of the chemotherapeutic compound.

320. The method of claim 316, wherein the effective amount of the chemotherapeutic compound and the effective amount of the SMO inhibitor have a Combination Index less than 1 as determined by Calcusyn software.

321. The method of claim 316, wherein the SMO inhibitor is selected from a compound of Table 3.

322. The method of claim 316, wherein the SMO inhibitor comprises BMS-833923, having the structure:

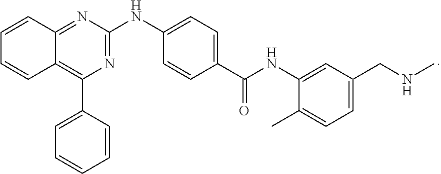 [SMILES: CNCc1ccc(C)c(NC(=O)c2ccc(Nc3nc(-c4ccccc4)c4ccccc4n3)cc2)c1]

323. The method of claim 322, wherein the BMS-833923 and chemotherapeutic agent are administered sequentially.

324. The method of claim 322, wherein administration of the chemotherapeutic agent to the subject commences before administration of the BMS-833923 to the subject.

325. The method of claim 322, wherein the chemotherapeutic agent is administered to the subject before the administration of the BMS-833923 to the subject.

326. The method of claim 322, wherein the chemotherapeutic agent is administered to the subject less than 48 hours prior to administering the BMS-833923 to the subject.

327. The method of claim 322, wherein the chemotherapeutic agent and the BMS-833923 are administered simultaneously to the subject.

328. The method of claim 322, wherein the chemotherapeutic agent is administered to the subject after administering the BMS-833923 to the subject.

329. The method of claim 322, wherein the BMS-833923 is administered at least about weekly.

330. The method of claim 322, wherein a dose of at least about 1 mg BMS-833923 is administered to the subject.

331. The method of claim 322, wherein the BMS-833923 is administered orally.

332. The method of claim 316, wherein the chemotherapeutic agent is selected from the group consisting cisplatin, carboplatin, nedaplatin, oxaliplatin, satraplatin, triplatin tetranitrate, taxol, gemcitabine, topotecan hydrochloride, doxorubicin and pegylated doxorubicin.

333. The method of claim 316, wherein the chemotherapeutic agent is administered at least weekly.

334. The method of claim 316, wherein the chemotherapeutic agent is administered intravenously.

335. The method of claim 316, wherein the cancer comprises a platinum resistant ovarian cancer cell.

336. The method of claim 316, wherein the cancer comprises an ovarian cancer cell.

337. The method of claim 316, wherein the cancer comprises an ovarian cancer stem cell.

338. A method for increasing the sensitivity of a cancer to a chemotherapeutic compound comprising contacting the cancer with an effective amount a SMO inhibitor and an effective amount of the chemotherapeutic compound, wherein the effective amount of the chemotherapeutic compound is significantly less than the effective amount of the chemotherapeutic compound in the absence of the SMO inhibitor.

339. The method of claim 338, wherein the chemotherapeutic agent comprises a platinum-based chemotherapeutic agent.

340. The method of claim 338, wherein the effective amount of the chemotherapeutic compound is at least about 10% less than the effective amount of the chemotherapeutic compound in the absence of the SMO inhibitor.

341. The method of claim 338, wherein the effective amount of the SMO inhibitor is an amount which significantly reduces the IC50 of the chemotherapeutic compound.

342. The method of claim 338, wherein the effective amount of the chemotherapeutic compound and the effective amount of the SMO inhibitor have a Combination Index less than 1 as determined by Calcusyn software.

343. The method of claim 338, wherein the SMO inhibitor is selected from a compound of Table 3.

344. The method of claim 338, wherein the SMO inhibitor comprises BMS-833923, having the structure:

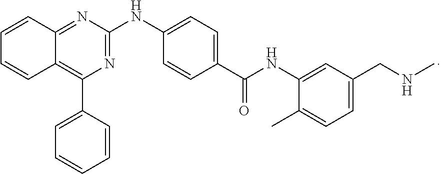 [SMILES: CNCc1ccc(C)c(NC(=O)c2ccc(Nc3nc(-c4ccccc4)c4ccccc4n3)cc2)c1]

345. The method of claim 344, wherein the BMS-833923 and chemotherapeutic agent are administered sequentially.

346. The method of claim 344, wherein administration of the chemotherapeutic agent to the subject commences before administration of the BMS-833923 to the subject.

347. The method of claim 344, wherein the chemotherapeutic agent is administered to the subject before the administration of the BMS-833923 to the subject.

348. The method of claim 344, wherein the chemotherapeutic agent is administered to the subject less than 48 hours prior to administering the BMS-833923 to the subject.

349. The method of claim 344, wherein the chemotherapeutic agent and the BMS-833923 are administered simultaneously to the subject.

350. The method of claim 344, wherein administration of the chemotherapeutic agent to the subject commences before administration of the BMS-833923 to the subject.

351. The method of claim 344, wherein the chemotherapeutic agent is administered to the subject after the administration of the BMS-833923 to the subject.

352. The method of claim 344, wherein the BMS-833923 is administered at least about weekly.

353. The method of claim 344, wherein a dose of at least about 1 mg BMS-833923 is administered to the subject.

354. The method of claim 344, wherein the BMS-833923 is administered orally.

355. The method of claim 338, wherein the chemotherapeutic agent is selected from the group consisting of cisplatin, carboplatin, nedaplatin, oxaliplatin, satraplatin, triplatin tetranitrate, taxol, gemcitabine, topotecan hydrochloride, doxorubicin and pegylated doxorubicin.

356. The method of claim 338, wherein the chemotherapeutic agent is administered at least weekly.

357. The method of claim 338, wherein the chemotherapeutic agent is administered intravenously.

358. The method of claim 338, wherein the cancer comprises a platinum resistant ovarian cancer cell.

359. The method of claim 338, wherein the cancer comprises an ovarian cancer cell.

360. The method of claim 338, wherein the cancer comprises an ovarian cancer stem cell.

361. A composition comprising a SMO inhibitor and a chemotherapeutic agent in a pharmaceutically acceptable carrier.

362. The composition of claim 361, wherein the SMO inhibitor is selected from a compound of Table 3.

363. The composition of claim 361, wherein the SMO inhibitor comprises BMS-833923, having the structure:

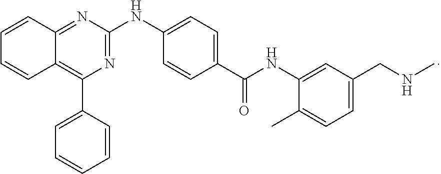 [SMILES: CNCc1ccc(C)c(NC(=O)c2ccc(Nc3nc(-c4ccccc4)c4ccccc4n3)cc2)c1]

364. The composition of claim 361, wherein the chemotherapeutic agent is selected from the group consisting of cisplatin, carboplatin, nedaplatin, oxaliplatin, satraplatin, triplatin tetranitrate, taxol, gemcitabine, topotecan hydrochloride, doxorubicin and pegylated doxorubicin.

365. The composition of claim 361 comprising a pill, tablet, powder or solution.
